(19) United States
(12) Patent Application Publication
(10) Pub. No.: US 2024/0124942 A1
(43) Pub. Date: Apr. 18, 2024

(54) MARKERS OF PREDICTION OF RESPONSE TO CAR T CELL THERAPY

(71) Applicants: FUNDACIÓ INSTITUT DE RECERCA CONTRA LA LEUCÈMIA JOSEP CARRERAS, Badalona (ES); BARCELONA SUPERCOMPUTING CENTER - CENTRO NACIONAL DE SUPERCOMPUTACIÓN, Barcelona (ES); INSTITUCIÓ CATALANA DE RECERCA I ESTUDIS AVANÇATS, Barcelona (ES)

(72) Inventors: Manel Esteller Badosa, Badalona (ES); Lorea Villanueva Legarda, Badalona (ES); Carlos Antonio García Prieto, Badalona (ES)

(21) Appl. No.: 18/547,904

(22) PCT Filed: Feb. 25, 2022
(86) PCT No.: PCT/EP2022/054803

§ 371 Date: Aug. 25, 2023

(30) Foreign Application Priority Data
Feb. 26, 2021 (EP) 21382168.9
Sep. 10, 2021 (EP) 21382815.5

(51) Int. Cl. C12Q 1/6886

(52) U.S. Cl. CPC C12Q 1/6886; C12Q 2600/106; C12Q 2600/154

(57) ABSTRACT

In vitro methods for predicting the response of a subject to autologous chimeric antigen receptor T-cell (CAR T-cell) therapy, the methods including determining the methylation status of one or more cytosines in CpG sites of CAR T-cells. Means and kits for carrying out the methods. With the determination of the methylation status of the one or more cytosines in CpG sites, a complete response free of events and long overall survival can be predicted as an outcome of the CAR T-cell therapy, in particular to subjects with B-cell malignancies.

This application claims the benefit of European Patent Application EP21382168.9 filed the 26^{th} of February 2021, and of European Patent Application EP21382815.5 filed the 10^{th} of September 2021.

### TECHNICAL FIELD

The invention relates to the field of signatures of CAR T cells and markers for determining the most probable outcome of a therapy with these cells. The invention also relates to methods of diagnostic and companion diagnostics, as well as to devices (kits) for the carrying out of the methods.

### BACKGROUND ART

Chimeric antigen receptor (CAR) T-cell therapy has proved to be effective in patients for whom few therapeutic options otherwise remained, such as those with relapsed/refractory (R/R) B-cell malignancies. The use of autologous, genetically modified T-cells targeting the CD19 antigen (CART19) in pediatric B-cell acute lymphoblastic leukemia (ALL) have yielded complete response rates in around 80% of cases overall, the percentage was lower in older patients, leading to frequent long-term remissions. In B-cell lymphomas, such as diffuse large B-cell lymphoma (DLBCL), adoptive cell transfer therapy is less successful, with approximately 30-40% of cases experiencing long-term remissions. These results have finally led to clinical approval of the commercial treatments with tisagenlecleucel (Kymriah), axicabtagene ciloleucel (Yescarta) and brexucabtagene autoleucel (Tecartus). Despite the great hopes that the use of CART19 cells has raised, treatment failure is not uncommon. Of note that these therapies are expensive and assuring their success is of high importance for the Health Systems. The discovery of predictive biomarkers of response and outcome to CART19 therapy would be highly significant for risk stratification, the selection of alternative approaches for resistant/non-responder patients, and for improving newly developed CART T-cell approaches. The lack of initial clinical response or the occurrence of relapse after CART19 treatment could be attributed to many possible causes related to the CART construct, the preparation of the infused cells, the delivery of the transduced cells, and the biological features of the targeted transformed cells. However, only a few defects associated with CART19 inefficacy have been identified, the most widely studied being tumor antigen escape by loss of the CD19 protein (see Majzner R G, Mackall C L., “Tumor antigen escape from CAR T-cell therapy”, Cancer Discov 2018; 8: 1219-26). Very few other candidate molecular biomarkers for predicting CART19 clinical response in pre-infused cells have been proposed.

Some examples include the CAR genomic integration site, as disclosed by Fraietta et al., in “Determinants of response and resistance to CD19 chimeric antigen receptor (CAR) T cell therapy of chronic lymphocytic leukemia”, Nat Med 2018; 24: 563-71; or by Fraietta et al., in “Disruption of TET2 promotes the therapeutic efficacy of CD19-targeted T cells”, Nature 2018; 558: 307-12; or by Nobles et al., in “CD19-targeting CART cell immunotherapy outcomes correlate with genomic modification by vector integration” J Clin Invest 2020; 130: 673-85. Other candidate molecular biomarkers relate to the expression of cytokines in CAR T cells, as disclosed by Rossi et al., in “Preinfusion polyfunctional anti-CD19 chimeric antigen receptor T cells are associated with clinical outcomes in NHL”, Blood 2018; 132: 804-14.

The international patent application WO2020092455 (The Broad Inst. Inc. et al.) discloses a method for selecting a candidate CAR T cell using a signature of gene expression. The document also discloses using these selected cells for treating cancer, and a mode to predict with the gene expression signature the outcome of the treatment. In a similar way, the international patent application WO2018209324 (The Broad Inst. Inc. et al.) proposes another signature of gene expression in T-cells (e.g. CAR T cells) to predict the outcome of the treatment with the same, as well as the expected overall survival.

All these previous methods, mainly based on gene signatures for detecting genes and/or polypeptides as gene expression outputs, imply the disadvantage of requiring reagent means of multiple type, such as specific hybridization probes, primers for amplification and/or sequencing, antibodies or fragments, aptamers, particular solvents or buffers for each reaction and accompanying detectable labels, and the corresponding control for each marker that is to be determined. This complexity derives generally in expensive and time-consuming tests when they are applied to clinics. Moreover, to assure a desired sensitivity with affordable and relatively fast times required in clinics, reagents of high quality are needed, also contributing to increasing the costs of the tests.

Thus, although there are some methods and biomarkers for predicting response to CAR T-cells treatment, there is still a need of other methods, which being reliable enough, do not imply the disadvantages of determining gene signatures (i.e., gene expression). Also there is a need of more informative methods, not only predicting the response but other important aspects of this response, such as the type of response (most probable outcomes), including for example the probability of remissions.

### SUMMARY OF INVENTION

Inventors surprisingly found out that a particular epigenetic profile observed in pre-infused CAR T-cells (in particular directed against CD19 (CART19)), and based on DNA methylation microarrays, was associated with complete clinical response (CR) and improved event-free survival (EFS) and overall survival (OS) in patients with B-cell malignancy who received the adoptive cell treatment. The DNA methylation study of the CAR T-cells (e.g. CART19 cells) also identified epigenetic loci associated with the common adverse effects of cytokine release syndrome (CRS) and immune effector cell-associated neurotoxicity syndrome (ICANS). The signature suggests that the clinical benefit of CAR T-cells (in particular CART19) therapy occurs mainly in infused products enriched in naive-like or early memory phenotype T-cells. Inventors found that, advantageously, the DNA methylation status of single genes associated with the regulation of protein levels, which may be determined with easily, was also of value as a predictor of the clinical benefit of the therapy to regulating protein levels.

According to the best of the inventor's knowledge, this is the first time a methylation profile of the cells to be infused gives relevant information in relation to the success of the therapy.

Thus, it is herewith disclosed in essence an in vitro method for predicting the response of a subject to autologous chimeric antigen receptor T-cell (CAR T-cell) therapy, the method comprising:
(a) determining the methylation status of one or more CpG sites of CAR T-cells, said CAR T-cells obtained from an isolated sample of the subject comprising T-cells that, once isolated, have been transduced with the CAR, and (b) comparing the methylation status of the one or more CpG sites with a reference value or range of values of response to CAR T-cell therapy, and determining the subject will respond to CAR T-cell therapy if the methylation status of the one or more CpG sites is equal to said reference value or within the range of reference values.

Thus, in a first aspect the invention relates to an in vitro method for predicting the response of a subject to autologous chimeric antigen receptor T-cell (CAR T-cell) therapy, the method comprising:
(a) determining the methylation status of one or more cytosines in CpG sites of CAR T-cells, said CAR T-cells obtained from an isolated sample of the subject comprising T cells that, once isolated, have been transduced with the CAR, the one or more cytosines in CpG sites of CAR T-cells selected from the group consisting of:
cytosine at position 86332162 of human chromosome 2, cytosine at position 188676237 of human chromosome 1; cytosine at position 105907265 of human chromosome 6; cytosine at position 234087867 of human chromosome 1; cytosine at position 32353565 of human chromosome 11; cytosine at position 22634199 of human chromosome 10: cytosine at position 45028225 of human chromosome 2; cytosine at position 220414164 of human chromosome 1; cytosine at position 209809 of human chromosome 6; cytosine at position 62905816 of human chromosome 1; cytosine at position 79780164 of human chromosome 6; and cytosine at position 28725934 of human chromosome 8, all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of response to CAR T-cell therapy, and determining the subject will respond to CAR T-cell therapy (i.e., that is a candidate to this autologous cell therapy) if the methylation status is of the one or more CpG sites equal to said reference value or within the range of reference values.

It is also herewith disclosed an in vitro method for predicting the response of a subject to an autologous chimeric antigen receptor T-cell (CAR T-cell) therapy, the method comprising:
(a) determining the methylation status of one or more CpG sites of CAR T-cells, said CAR T-cells obtained from an isolated sample of the subject comprising T-cells that, once isolated, have been transduced with the CAR, the one or more CpG sites of CAR T-cells selected from the group consisting of:
cytosine at position 86332162 of human chromosome 2, cytosine at position 188676237 of human chromosome 1; cytosine at position 45028225 of human chromosome 2; cytosine at position 220414164 of human chromosome 1; cytosine at position 209809 of human chromosome 6; cytosine at position 62905816 of human chromosome 1; cytosine at position 79780164 of human chromosome 6 and cytosine at position 28725934 of human chromosome 8, all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of response to CAR T-cell therapy and determining the subject will respond to CAR T-cell therapy (i.e., that is a candidate to this autologous cell therapy) if the methylation status of the one or more CpG sites is equal to said reference value or within the range of reference values.

Most of the genes associated with these DNA methylation loci (cytosines in the CpG sites) are involved in regulating protein levels, as will be detailed in next sections.

As will be illustrated in the examples below, the carrying out of this method gives valuable information regarding the most probable outcome of the response to the therapy, namely a complete response (CR), which means than no remissions will take place. The main study outcomes were improved event-free survival (EFS) and overall survival (OS), which are parameters assuring the therapy will succeed. Thus, using these sites linked to CR, particular signature (termed in this description EPICART and EPICART18) were established, which were associated with CR and enhanced EFS and OS.

Advantageously, the determining of the methylation of these one or more CpG sites is uniformly performed with particular reagents and technologies for methylation determination. Thus, proposed method implies the advantage in relation with other method determining other signatures, such as gene expression, that a simpler methodology is involved for the determination of methylation.

Moreover, accuracy according to operating characteristic (ROC) curve was high (Area Under Curve [AUC] mean=0.91, 95% Cl=0.85-0.97 for EPICART, and AUC value of about 0.8 for EPICART18).

All these advantages of the new method of predicting response to CAR T-cell therapy encourage its application, since it is assured its success in a subject in need thereof (e.g. subjects suffering from B-cell malignancy).

Once the outcome of the possible therapy is determined, it can be decided if the same is recommended or not for that particular subject. Thus, another aspect of the invention (second) is a method of deciding and/or recommending whether to initiate a CAR T-cell therapy for a subject suffering from B-cell malignancies, which method comprises carrying out the in vitro method as defined in the first aspect; and wherein if the subject is determined to respond to CAR T-cell therapy, then this therapy is recommended. Thus, the invention relates to a method of deciding and/or recommending whether to initiate a CAR T-cell therapy for a subject suffering from B-cell malignancies, which method comprises (a) determining the methylation status of one or more CpG sites of CAR T-cells previously transduced after isolation from a subject, said CpG sites selected from the list indicated for the first aspect, and (b) comparing the methylation status of the one or more CpG sites with a reference value or range.

There have been proposed in the field, methods for obtaining T-cells with improved differentiation potential, which is finally translated with an increased therapy efficacy when used in adoptive cell therapy. An increased therapy efficacy is different than a prediction of response, much less the prediction of a complete response with a very good outcome. One example of a methods for obtaining T-cells with improved differentiation potential is disclosed in the international patent application WO2020170231 (St. Jude's Children Research Institute). The determination of this state of differentiation is done by means of the analysis of the methylation status of certain CpG sites. Based on this methylation signature defining therapy efficacy, inventors in WO2020170231 propose then T-cell populations (e.g., CAR T-cells) with modulated methylation profiles to be used in the therapy. This “modulated methylation profiles” are the result of combining at least two populations having an increased differentiation potential based on a multipotency score determined by the methylation status of the each of the populations measured independently.

If previously to the preparation of this combination of populations with increased differentiation profiles in the case of CAR T-cells, they are tested to see if they correspond moreover to a respondent signature, even an improved therapy performance is provided.

Thus, in a third aspect, the invention relates, moreover, to a method of modulating methylation profiles in CpG sites of CAR T-cells, the method comprising the step of first carrying out the method as defined in the first aspect; and further modulating methylation profiles related with the differentiation and/or efficacy of therapy, said modulating carried out by means of methods as disclosed by previous authors and known by the skilled person in the art.

Inventors also propose a method of directly modifying the methylation status in CpG sites of CAR T cells, the method comprising the step of first carrying out the method as defined in the first aspect; and further modifying methylation status to obtain a methylation profile corresponding to response to therapy with CAR T-cells.

T-cell populations obtainable by all these methods are then included in pharmaceutical compositions which comprise them and one or more pharmaceutically acceptable excipients.

Another aspect of the invention is also the use of means comprising DNA oligonucleotides suitable for determining DNA methylation status of one or more CpG site cytosines, for predicting the response of a subject to chimeric antigen receptor T cell (CAR T cell) therapy, in any of the methods of the first and second aspects.

### BRIEF DESCRIPTION OF DRAWINGS

FIG. 1 shows a graphic scheme with the experimental design developed to detect DNA methylation changes in patient T-cells upon CAR transduction.

FIG. 2 shows the Complete response and DNA methylation predictive signature (EPICART) association with event-free survival (EFS) and overall survival (OS) in patients with B-cell malignancy treated with CART19 therapy. (A) Kaplan-Meier analysis of EFS (left) and OS (right) in 34 B-cell malignancy patients according to the presence of complete response or its absence (partial response [PR]+stable disease [SD]+progression of the disease [PD]). (B) Kaplan-Meier analysis of EFS (left) and OS (right) in the same B-cell malignancy patients according to the presence EPICART signature in the pre-infused CART19 cells, defined by the methylation status of the 32 CpG sites associated with CR (EPICART-positive [+] signature). For all cases, P was calculated using the log-rank function. Univariate Cox regression analysis is represented as the hazard ratio (HR) with a 95% confidence interval (95% CI). Values of P<0.05 were considered to be statistically significant. The number of events is also shown.

FIG. 3 (A-G) depicts the Kaplan-Meier estimates of EFS with respect to the CART19 cell pre-infusion methylation status of seven single CpG loci associated with complete clinical response in B-cell malignancy patients treated with the adoptive cell therapy. P was calculated using the log-rank function. Univariate Cox regression analysis is represented as the hazard ratio (HR) with a 95% confidence interval (95% CI). Values of P<0.05 were considered to be statistically significant.

FIG. 4 (A-G) depicts the Kaplan-Meier estimates of OS relative to the CART19 cell pre-infusion methylation status of the seven single CpG loci of FIG. 3 in patients with B-cell malignancy treated with adoptive cell therapy. P was calculated using the log-rank function. Univariate Cox regression analysis is represented as the hazard ratio (HR) with a 95% confidence interval (95% CI). Values of P<0.05 were considered to be statistically significant.

FIG. 5, related to Example 2, shows a Complete response and DNA methylation signature (EPICART18) associated with event-free survival (EFS) and overall survival (OS) in the discovery cohort of patients with B-cell malignancy treated with CART19 therapy. (A) Kaplan-Meier analysis of EFS (left) and OS (right) in 79 B-cell malignancy patients according to the presence of complete response or its absence (partial response [PR]+stable disease [SD]+progression of the disease [PD]). (B) Kaplan-Meier analysis of EFS (left) and OS (right) in the same B-cell malignancy patients according to the presence EPICART18 signature in the pre-infused CART19 cells, defined by the methylation status of the 18 CpG sites associated with CR (EPICART18-positive [+] signature, simplified in FIG and indicated as EPICART). For all cases, P was calculated using the log-rank function. Univariate Cox regression analysis is represented as the hazard ratio (HR) with a 95% confidence interval (95% CI). Values of P<0.05 were considered to be statistically significant.

FIG. 6, related to Example 2, shows the Complete response and EPICART18 signature (abbreviated in FIG as EPICART) associated with EFS and OS in the validation cohort of patients with B-cell malignancy treated with CART19 therapy. (A) Kaplan-Meier analysis of EFS (left) and OS (right) in 35 B-cell malignancy patients according to the presence of complete response or its absence (partial response [PR]+stable disease [SD]+progression of the disease [PD]). (B) Kaplan-Meier analysis of EFS (left) and OS (right) in the same B-cell malignancy patients according to the presence EPICART18 signature in the pre-infused CART19 cells, defined by the methylation status of the 18 CpG sites associated with CR (EPICART-positive [+] signature). For all cases, P was calculated using the log-rank function. Univariate Cox regression analysis is represented as the hazard ratio (HR) with a 95% confidence interval (95% CI). Values of P<0.05 were considered to be statistically significant.

FIG. 7 (A-F), related to Example 2, depict Kaplan-Meier estimates of EFS with respect to the CART19 cell pre-infusion methylation status of six candidate single CpG loci in B-cell malignancy patients treated with the adoptive cell therapy. P was calculated using the log-rank function. Univariate Cox regression analysis is represented as the hazard ratio (HR) with a 95% confidence interval (95% CI). Values of P<0.05 were considered to be statistically significant.

FIG. 8 (A-F), related to Example 2, depict Kaplan-Meier estimates of OS relative to the CART19 cell pre-infusion methylation status of six candidate single CpG loci in patients with B-cell malignancy treated with adoptive cell therapy. P was calculated using the log-rank function. Univariate Cox regression analysis is represented as the hazard ratio (HR) with a 95% confidence interval (95% CI). Values of P<0.05 were considered to be statistically significant.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

In the sense of the present invention, the expression “DNA methylation signature”, or “DNA methylation status” (used herewith as synonymous expressions) relates to the qualitative and quantitative methylation in a particular nucleotide, nucleotide sequence or sequence set (group of sequences). DNA methylation is a biochemical process where a methyl group is added to the cytosine or adenine DNA nucleotides. Most particularly, methylation is usually found in CpG islands, which are regions with a high frequency of CpG sites. CpG sites or CG sites are also regions of DNA (or DNA genomic sequences) where a cytosine nucleotide occurs next to a guanine nucleotide in the linear sequence of bases along its length. Cytosines in CpG dinucleotides can be methylated to form 5-methylcytosine. The CpG sites are well-defined genomic regions and they are indexed in database giving to said regions an identification number (ID) as cg_number, according to Illuminae's CpG Loci Identification wherein flanking sequences regions around the CpG dinucleotide are used to generate unique CpG cluster IDs (cg_number). Thus, in a particularized mode, the term “methylation status of one or more CpG sites” relates to the presence, absence, and/or quantity of methylation at certain cytosines of each identified CpG sites. The methylation status of DNA and in particular of any CpG site can optionally be represented or indicated by a “methylation value” or “methylation level.” A methylation value, score or level can be generated, for example, by quantifying the methylation in a cytosine using, for example a β-value ranging from 0 to 1, by means of formula (I):

β-value_{Cyt}=max(y_{methCyt},0)/[max(y_{unmethCyt},0)+max(y_{methCyt},0)] (I),

wherein max y_{methCyt} is the maximal signal intensity detected for a methylated cytosine in the CpG site set; and max y_{unmethCyt} is the maximal signal detected for an unmethylated cytosine in the CpG site or set of PcP sites. For each CpG site a particular cut-off can be fixed for deciding if a differential methylation in this site exists or not. The methylation score, or level of the two or more of the CpG sites can in addition be computerized in complex formulas or algorithms to obtain indexes of methylation, which can also be used to take decisions regarding the methylation status of a sample and then stablish the investigated (interrogated) correlation, such as the probability of response to a therapy. For example, one or more CpG sites can be used as input to a machine learning algorithm to calculate a respondent signature or index. For example, in certain instances, one-class logistic regression can be used to obtain the respondent index. Further examples of widely used machine learning methods, algorithms, computer programs, or systems that can be applied herein include, but are not limited to, are Neural network (multi-layer perceptron), Support vector machines, k-nearest neighbors, Gaussian mixture model, Gaussian, naive Bayes, Decision tree, and RBF classifier. In some embodiments, the Respondent index is generated using Linear classifiers (for e.g., partial least squares determinant analysis (PLS-DA), Fisher's linear discriminant, Logistic regression (eg., one-class logistic regression), Naive Bayes classifier, Perceptron), Support vector machines (for e.g., least squares support vector machines), quadratic classifiers, Kernel estimation (for e.g., k-nearest neighbor), Boosting, Decision trees (for e.g., Random forests), Neural networks, Bayesian networks, Hidden Markov models, or Learning vector quantization.

The terms “measuring” and “determining” are used interchangeably throughout, and refer to methods which include obtaining a subject sample and/or detecting the methylation status or level of a biomarker(s) (i.e. cytosine methylation in CpG sites) in a sample. In this description when it is indicated that the methylation status of one or more CpG site is determined is to be understood that the methylation in the indicated cytosine of the CpG site is determined. Therefore, the expressions “cytosine in CpG site” or “CpG site” are used interchangeably too.

The term “reference value” or “reference interval”, as used herein, relates to a predetermined criteria used as a reference for evaluating the values or data obtained from the samples collected from a subject, and, in this particular case obtained from CAR transduced T-cells. The reference value or reference level can be an absolute value; a relative value; a value that has an upper or a lower limit; a range of values (reference interval); an average value; a median value, a mean value, or a value as compared to a particular control or baseline value. A reference value can be based on an individual sample value, such as for example, a value obtained from a sample from the subject being tested, but at an earlier point in time. The reference value can be based on a large number of samples, such as from population of subjects of the chronological age matched group, or based on a pool of samples including or excluding the sample to be tested. Reference values have been determined for the methylation status of the one or more CpG sites. Range of values of each CpG sites and particular combinations of the values of the different CpG sites provide for correct classification of subjects with high sensitivity and specificity.

The term “complete response (CR)” relates to the full working of the CAR T-cell therapy, which in clinical terms is translated with no remission of the diseases the therapy was administered for. It is defined as opposed to a “partial response (PR)”, or to a “stable disease (SD)” or to a “progression of the disease (PD)”. Partial response means a reduction in the extent of cancer in response to therapy without reaching a complete remission of the disease, stable disease includes a type of response in which the subject still suffers from the disease, but it does not evolve to a worse outcome; and progression of the disease means that the disease evolves to a worse scenario than initially prior to the therapy, although this worsening is not to be directly related with the therapy (i.e. the therapy simply did not work).

The term “Overall survival (OS)” refers to the length of time from either the date of diagnosis or the start of treatment for a disease, such as cancer, that patients diagnosed with the disease are still alive. In a clinical trial, measuring the overall survival is one way to see how well a new treatment works.

The term “event free survival (EFS)” corresponds, in cancer, to the length of time after primary treatment for a cancer ends that the patient remains free of certain complications or events that the treatment was intended to prevent or delay. These events may include the return of the cancer or the onset of certain symptoms, such as bone pain from cancer that has spread to the bone. In a clinical trial, measuring the event-free survival is one way to see how well a new treatment works.

In the particular case of this description, when relating to CAR T-cell therapy against CD19 antigen (CART19), the EFS was defined as the time from the start of CART19 treatment until the first occurrence of progression, relapse, or death. Overall survival (OS) was defined as the time from the start of CART19 treatment until death.

As previously indicated, the first aspect of the invention is an in vitro method for predicting the response of a subject to autologous chimeric antigen receptor T cell (CAR T cell) therapy, the method comprising:
(a) determining the methylation status of one or more CpG sites of CAR T-cells, said CAR T cells obtained from an isolated sample of the subject comprising T cells that, once isolated, have been transduced with the CAR, the one or more CpG sites of CAR T cells selected from the group consisting of: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); cytosine at position 22634199 of human chromosome 10 (cg12610471): cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177) and cytosine at position 28725934 of human chromosome 8 (cg08544307), all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of response to CAR T-cell therapy, and determining the subject will respond to CAR T-cell therapy (i.e., that is a candidate to this autologous cell therapy) if the methylation status is of the one or more CpG sites equal to said reference value or within the range of reference values.

In a particular embodiment of the first aspect of the invention the in vitro method for predicting the response of a subject to autologous chimeric antigen receptor T cell (CAR T cell) therapy, the method comprises:
(a) determining the methylation status of one or more CpG sites of CAR T-cells, said CAR T cells obtained from an isolated sample of the subject comprising T cells that, once isolated, have been transduced with the CAR, the one or more CpG sites of CAR T cells selected from the group consisting of:
cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177) and cytosine at position 28725934 of human chromosome 8 (cg08544307), all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of response to CAR T cell therapy, and determining the subject will respond to CAR T cell therapy if the methylation status of the one or more CpG sites is equal to said reference value or within the range of reference values.

The identifications indicated as cg_number into brackets in previous paragraph, correspond, as indicated before, to the indexed in database univocal identification number (ID) of the sequence of the probe that allows identification of the cytosine of interest in the CpG site, according to Illuminae's CpG Loci Identification. With this CpG Loci Identification, flanking sequences regions around the CpG dinucleotide containing the cytosine of interest are used to generate unique CpG cluster IDs (cg_number). These cg-numbers into brackets are also indicated for the listed cytosines in the CpG loci throughout this description in the next paragraphs.

In a particular embodiment of the first aspect, the methylation status is determined in one, two, three, four, five, six, seven or the eight indicated cytosines.

With the combination of two or more of the CpG sites the accuracy of the method of determining response can be modulated. In any case, with only one of the methylation statuses in said cytosines of the CpG sites equal to said reference value or within the range of reference values of a responder profile, the sample isolated from the subject and further transduced with the CAR is considered of a respondent subject.

In a more particular embodiment of the first aspect, the methylation status of the following CpG sites of CAR T cells is determined: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); and cytosine at position 22634199 of human chromosome 10 (cg12610471).

Each one of these six markers are individually associated with both significant extended EFS and long OS. Thus, inventors propose these six epigenomic loci that, analysed alone, are associated with better EFS and OS. Moreover, these six loci, also individually were associated to a complete response. Thus, the determination of the methylation status of these six cytosines (i.e., the panel of six loci) supposes a simplified method to fast known if the subject will respond to the autologous CAR T-cell therapy.

In a more particular embodiment, complete response to CAR T-cell therapy is determined and a high/extended EFS and OS when:
one or more of cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216), are found to be methylated; and/or cytosine at position 22634199 of human chromosome 10 (cg12610471) is found to be unmethylated.

Table A below correlates/associates these six loci within identified genes or not. The four genes associated with these six DNA methylation loci were PTCD3 and POLR1A, involved in protein production regulation at ribosomes; SLC35F3, a thiamine transferase involved in T-cell infiltration; and SPAG6 that regulates cell apoptosis through the TRAIL signaling. SPAG6 was further studied, given the proposed used of a TRAIL-variant to overcome CAR-T resistance and the CpG location at the transcription start site. Hypermethylation-associated silencing was also found for PTCD3, the other candidate gene with an identified differentially methylated CpG site in its promoter region.

TABLE A
Annotation of the 6 CpGs correlated with Complete Response and with significant
improvement in event-free survival (EFS) and overall survival (OS).

| | | | Complete | | |
|---|---|---|---|---|---|
| | | | Response | | |
| | Chromosomal | Associated | FDR | EFS | OS |
| Probe ID | position (hg19) | gene | p-value | p-value | p-value |
| cg12012941 | chr1: 188676237 | Not described | 0.0007 | 0.011 | 0.01 |
| cg04267686 | chr6: 105907265 | Not described | 0.001 | 0.016 | 0.001 |
| cg25534076 | chr1: 234087867 | SLC35F3 | 0.002 | 0.04 | 0.029 |
| cg12260379 | chr2: 86332162 | PTCD3; POLR1A | 0.012 | 0.028 | 0.037 |
| cg09992216 | chr11: 32353565 | Not described | 0.014 | 0.009 | 0.004 |
| cg12610471 | chr10: 22634199 | SPAG6 | 0.024 | 0.001 | 0.003 |

Annotation retrieved from the Infinium MethylationEPIC Array manifest. Probe ID: unique identifier from the Illumina CG database. Chromosomal position (hg19): chromosomal coordinates of the CpG (build hg19). Associated gene: target gene name from the UCSC database. The False Discovery Rate (FDR) adjusted p-value of the Complete Response is derived from the Fisher's exact test (CR vs NR/SD/PD). The p-value of event-free survival (EFS) and overall survival (OS) is derived from the log-rank test in Kaplan-Meier curves.

In a more particular embodiment, optionally in combination with any of the embodiments above or below, when one or more of the previously six listed cytosines (i.e., one, two, three, four, five or the six) are determined, the in vitro method further comprises determining the methylation status of one or more CpG sites of CAR T-cells, said CAR T-cells obtained from an isolated sample of the subject comprising T-cells that, once isolated, have been transduced with the CAR, the one or more CpG sites of CAR T-cells selected from the group consisting of: cytosine at position 95139986 of human chromosome 10 (cg10039734); cytosine at position 127612751 of human chromosome 6 (cg25571136); cytosine at position 131058184 of human chromosome 2 (cg01311063); cytosine at position 90081872 of human chromosome 14 (cg12504912); cytosine at position 123944014 of human chromosome 12 (cg10236435); cytosine at position 134457731 of human chromosome 10 (cg25268100); cytosine at position 46993515 of human chromosome 10 (cg25995980); cytosine at position 209809 of human chromosome 6 (cg15253304); cytosine at position 122144477 of human chromosome 2 (cg17511575); cytosine at position 6643814 of human chromosome 6 (cg09367268); cytosine at position 60877850 of human chromosome 18 (cg11416737); and cytosine at position 42299379 of human chromosome 19 (cg24267358), all cytosine positions in human chromosomes according to the chromosome map and sequence entries of database UCSC Genome Browser on Human February 2009, GRCh37/hg19 assembly of the University of California Santa Cruz (UCSC).

In a more particular embodiment, the method comprises determining the methylation status in one, two, three, four, five, six, seven, eight, nine, ten, eleven, or twelve of the indicated cytokines in the previous paragraph.

The addition of any one of these twelve-cytosine methylation status to the first six indicated, could improve the accuracy of the method.

Indeed, in yet another more particular embodiment of the first aspect, the methylation status of the cytosines in the following 18 CpG sites of CAR T cells is determined: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); cytosine at position 22634199 of human chromosome 10 (cg12610471); cytosine at position 95139986 of human chromosome 10 (cg10039734); cytosine at position 127612751 of human chromosome 6 (cg25571136); cytosine at position 131058184 of human chromosome 2 (cg01311063); cytosine at position 90081872 of human chromosome 14 (cg12504912); cytosine at position 123944014 of human chromosome 12 (cg10236435); cytosine at position 134457731 of human chromosome 10 (cg25268100); cytosine at position 46993515 of human chromosome 10 (cg25995980); cytosine at position 209809 of human chromosome 6 (cg15253304); cytosine at position 122144477 of human chromosome 2 (cg17511575); cytosine at position 6643814 of human chromosome 6 (cg09367268); cytosine at position 60877850 of human chromosome 18 (cg11416737); and cytosine at position 42299379 of human chromosome 19 (cg24267358).

Indeed, these panel of 18 markers include all the cytosines which methylation status (methylated or unmethylated) correlate with a complete response. Interestingly, when this 18 CpG sites panel is used to obtain a classification model it provides an epigenetic signature (referred to hereafter as the EPICART18 signature) with clinical value, as will be illustrated in examples and figures below. A positive signature (named EPICART18+), which means that differential methylation exists in these CpG sites prior and after transduction of the cells, is associated with an improved or high Event free survival (EFS) and overall survival (OS).

Next Table B correlates each one of the 18 cytosines in CpG sites with an associated gene or not, as previously commented for the panel of 6.

TABLE B
Annotation of the 18 CpG sites correlated with complete response

| | Chromosomal | Associated | Complete Response |
|---|---|---|---|
| Probe ID | position (hg19) | gene | FDR p-value |
| cg12012941 | chr1: 188676237 | Not described | 0.001 |
| cg04267686 | chr6: 105907265 | Not described | 0.001 |
| cg25534076 | chr1: 234087867 | SLC35F3 | 0.002 |
| cg10039734 | chr10: 95139986 | MYOF | 0.007 |
| cg25571136 | chr6: 127612751 | ECHDC1 | 0.007 |
| cg01311063 | chr2: 131058184 | Not described | 0.012 |
| cg12260379 | chr2: 86332162 | PTCD3;POLR1A | 0.012 |
| cg12504912 | chr14: 90081872 | FOXN3 | 0.012 |
| cg10236435 | chr12: 123944014 | SNRNP35 | 0.013 |
| cg09992216 | chr11: 32353565 | Not described | 0.014 |
| cg25268100 | chr10: 134457731 | INPP5A | 0.014 |
| cg25995980 | chr10: 46993515 | GPRIN2 | 0.014 |
| cg12610471 | chr10: 22634199 | SPAG6 | 0.024 |
| cg15253304 | chr6: 209809 | Not described | 0.024 |
| cg17511575 | chr2: 122144477 | CLASP1 | 0.024 |
| cg09367268 | chr6: 6643814 | LY86 | 0.026 |
| cg11416737 | chr18: 60877850 | BCL2 | 0.038 |
| cg24267358 | chr19: 42299379 | CEACAM3 | 0.041 |

Annotation retrieved from the Infinium MethylationEPIC Array manifest. Probe ID: unique identifier from the Illumina CG database. Chromosomal position (hg19): chromosomal coordinates of the CpG (build hg19). Associated gene: target gene name from the UCSC database. The False Discovery Rate (FDR) adjusted p-value of the Complete Response is derived from the Fisher's exact test (CR vs NR/SD/PD).

When a positive signature derived from the panel of the 6 or of the 18 CpG sites is established, the subject is considered that will completely respond to the therapy.

In a particular embodiment of the first aspect, the in vitro method allows determining if the response of a subject to an autologous CAR T-cell therapy is a complete response, which means that no remission of the disease is observed after therapy.

Also as previously indicated, in a particular embodiment of the first aspect, the method comprises determining the methylation status of one or more CpG sites selected from the group consisting of:
cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177) and cytosine at position 28725934 of human chromosome 8 (cg08544307).

In yet another more particular embodiment of the previous embodiment of the first aspect, the methylation status of the following CpG sites of CAR T cells is determined: cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); and cytosine at position 79780164 of human chromosome 6 (cg13554177).

Each one of these seven markers are individually associated with both significant extended EFS and long OS. Thus, inventors propose these seven epigenomic loci that, analysed alone, are associated with better EFS and OS. Moreover, these seven loci, also individually were associated to a complete response. Thus, the determination of the methylation status of these seven cytosines (i.e., the panel of seven loci) supposes a simplified method to fast known if the subject will respond to the autologous CAR T-cell therapy.

In a more particular embodiment, complete response to CAR T-cell therapy is determined and a high/extended EFS and OS when:
one or more of cytosine at position 86332162 of human chromosome 2 (cg12260379), cytosine at position 188676237 of human chromosome 1 (cg12012941), cytosine at position 220414164 of human chromosome 1 (cg04458195); cytosine at position 209809 of human chromosome 6 (cg15253304); cytosine at position 62905816 of human chromosome 1 (cg22171055), and cytosine at position 79780164 of human chromosome 6 (cg13554177) are found to be methylated; and/or cytosine at position 45028225 of human chromosome 2 (cg03593578) is found to be unmethylated.

Table 3 below correlates/associates these seven loci within identified genes or not. Bolded cytosines relate to these seven. These seven cytosines include five genes. It is of note that the five genes associated with these seven DNA methylation loci are involved in regulating protein levels. Thus, at least the one or more CpG sites associated with genes are cytosines in CpG sites selected from genes regulating protein levels. Thus, for example, USP1, RAB3GAP2, and PHIP were involved in protein degradation by the ubiquitin pathway, and PTCD3 and POLR1A played a role in protein production at the ribosomes. The case of USP1 could be particularly relevant because it controls the protein expression levels of Inhibitor of DNA Binding 2 (ID2),30 a gene that is overexpressed in the CD8 T-cells of infused CART19 patients who do not achieve a complete clinical response.

In another more particular embodiment of the first aspect, when the one or more of the previous CpG loci in the set of seven are determined, and optionally in combination with any of the embodiments above or below, the in vitro method further comprises determining the methylation status of one or more CpG sites of CAR T cells, said CAR T cells obtained from an isolated sample of the subject comprising T cells that, once isolated, have been transduced with the CAR, the one or more CpG sites of CAR T cells selected from the group consisting of: cytosine at position 134457731 of human chromosome 10 (cg25268100); cytosine at position 127612751 of human chromosome 6 (cg25571136), cytosine at position 6643814 of human chromosome 6 (cg09367268); cytosine at position 42299379 of human chromosome 19 (cg24267358); cytosine at position 32353565 of human chromosome 11 (cg09992216); cytosine at position 234087867 of human chromosome 1 (cg25534076); cytosine at position 105907265 of human chromosome 6 (cg04267686); cytosine at position 22634199 of human chromosome 10 (cg12610471); cytosine at position 95870440 of human chromosome 15 (cg18739950); cytosine at position 104470719 of human chromosome 10 (cg12700402); cytosine at position 43253559 of human chromosome 22 (cg01029450); cytosine at position 122144477 of human chromosome 2 (cg17511575); cytosine at position 131166906 of human chromosome 12 (cg26098972); cytosine at position 68481342 of human chromosome 16 (cg05948940); cytosine at position 100879199 of human chromosome 15 (cg07199183); cytosine at position 95139986 of human chromosome 10 (cg10039734); cytosine at position 183063459 of human chromosome 4 (cg19759671); cytosine at position 180614858 of human chromosome 5 (cg25606201); cytosine at position 134571377 of human chromosome 10 (cg27196695); cytosine at position 3600764 of human chromosome 12 (cg11596580); cytosine at position 90081872 of human chromosome 14 (cg12504912); cytosine at position 133000178 of human chromosome 12 (cg09698465); cytosine at position 46993515 of human chromosome 10 (cg25995980); cytosine at position 19229767 of human chromosome 9 (cg13469590); and cytosine at position 24229300 of human chromosome 1 (cg24452347), all cytosine positions in human chromosomes according to the chromosome map and sequence entries of database UCSC Genome Browser on Human February 2009, GRCh37/hg19 assembly of the University of California Santa Cruz (UCSC).

In a more particular embodiment, the method comprises determining the methylation status in one, two, three, four, five, six, seven, eight, nine, ten, eleven, twelve, thirteen, fourteen, fifteen, sixteen, eighteen, nineteen, twenty, twenty-one, twenty-two, twenty-three, twenty-four or the twenty-five of the indicated cytokines.

The addition of any one of these twenty-five-cytosine methylation status to the first eight indicated, and in particular to the panel of seven particularized in a previous embodiment, could improve the accuracy of the method.

Indeed, in a more particular embodiment of the first aspect, the in vitro method comprises determining the methylation status of the following 32 CpG sites of the CAR T cells derived from the isolated sample from the subject: cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177); cytosine at position 134457731 of human chromosome 10 (cg25268100); cytosine at position 127612751 of human chromosome 6 (cg25571136), cytosine at position 6643814 of human chromosome 6 (cg09367268); cytosine at position 42299379 of human chromosome 19 (cg24267358); cytosine at position 32353565 of human chromosome 11 (cg09992216); cytosine at position 234087867 of human chromosome 1 (cg25534076); cytosine at position 105907265 of human chromosome 6 (cg04267686); cytosine at position 22634199 of human chromosome 10 (cg12610471); cytosine at position 95870440 of human chromosome 15 (cg18739950); cytosine at position 104470719 of human chromosome 10 (cg12700402); cytosine at position 43253559 of human chromosome 22 (cg01029450); cytosine at position 122144477 of human chromosome 2 (cg17511575); cytosine at position 131166906 of human chromosome 12 (cg26098972); cytosine at position 68481342 of human chromosome 16 (cg05948940); cytosine at position 100879199 of human chromosome 15 (cg07199183); cytosine at position 95139986 of human chromosome 10 (cg10039734); cytosine at position 183063459 of human chromosome 4 (cg19759671); cytosine at position 180614858 of human chromosome 5 (cg25606201); cytosine at position 134571377 of human chromosome 10 (cg27196695); cytosine at position 3600764 of human chromosome 12 (cg11596580); cytosine at position 90081872 of human chromosome 14 (cg12504912); cytosine at position 133000178 of human chromosome 12 (cg09698465); cytosine at position 46993515 of human chromosome 10 (cg25995980); cytosine at position 19229767 of human chromosome 9 (cg13469590); and cytosine at position 24229300 of human chromosome 1 (cg24452347).

Indeed, these panel of 32 markers include all the cytosines which methylation status (methylated or unmethylated) correlate with a complete response. Interestingly, when we use this 32 CpG sites to obtain a classification model it provides an epigenetic signature (referred to hereafter as the EPICART signature) with clinical value, as will be illustrated in examples and figures below. A positive signature (named EPICART+), which means that differential methylation exists in these CpG sites prior and after transduction of the cells, is associated with an improved or high Event free survival (EFS) and overall survival (OS).

Next Table 3 correlates each one of the 32 cytosines in CpG sites with an associated gene or not, as previously commented.

TABLE 3
Annotation of the 32 CpGs correlated with Complete Response.

| | | | Complete |
|---|---|---|---|
| | Chromosomal position | Associated | Response |
| Probe ID | (hg19) | gene | P-value |
| cg25268100 | chr10: 134457731 | INPP5A | <0.0001 |
| cg12260379 | chr2: 86332162 | PTCD3, POLR1A | 0.0001 |
| cg25571136 | chr6: 127612751 | ECHDC1 | 0.0001 |
| cg09367268 | chr6: 6643814 | LY86 | 0.0002 |
| cg24267358 | chr19: 42299379 | CEACAM3 | 0.0002 |
| cg09992216 | chr11: 32353565 | Not described | 0.0004 |
| cg12012941 | chr1: 188676237 | Not described | 0.0009 |
| cg25534076 | chr1: 234087867 | SLC35F3 | 0.0009 |
| cg04267686 | chr6: 105907265 | Not described | 0.0028 |
| cg12610471 | chr10: 22634199 | SPAG6 | 0.0054 |
| cg18739950 | chr15: 95870440 | Not described | 0.0063 |
| cg12700402 | chr10: 104470719 | ARL3 | 0.0067 |
| cg01029450 | chr22: 43253559 | ARFGAP3 | 0.0069 |
| cg17511575 | chr2: 122144477 | CLASP1 | 0.0069 |
| cg26098972 | chr12: 131166906 | Not described | 0.0069 |
| cg03593578 | chr2: 45028225 | Not described | 0.0101 |
| cg05948940 | chr16: 68481342 | SMPD3 | 0.0101 |
| cg07199183 | chr15: 100879199 | ADAMTS17 | 0.0101 |
| cg10039734 | chr10: 95139986 | MYOF | 0.0143 |
| cg04458195 | chr1: 220414164 | RAB3GAP2 | 0.0161 |
| cg15253304 | chr6: 209809 | Not described | 0.0161 |
| cg 19759671 | chr4: 183063459 | MGC45800 | 0.0171 |
| cg22171055 | chr1: 62905816 | USP1 | 0.0175 |
| cg25606201 | chr5: 180614858 | Not described | 0.0181 |
| cg27196695 | chr10: 134571377 | INPP5A | 0.0244 |
| cg11596580 | chr12: 3600764 | PRMT8 | 0.033 |
| cg13554177 | chr6: 79780164 | PHIP | 0.0346 |
| cg12504912 | chr14: 90081872 | FOXN3 | 0.0369 |
| cg09698465 | chr12: 133000178 | Not described | 0.0427 |
| cg25995980 | chr10: 46993515 | GPRIN2 | 0.0447 |
| cg13469590 | chr9: 19229767 | Not described | 0.0453 |
| cg24452347 | chr1: 24229300 | CNR2 | 0.0454 |

Annotation retrieved from the Infinium MethylationEPIC Array manifest. Probe ID: unique identifier from the Illumina CG database. Chromosomal position (hg19): chromosomal coordinates of the CpG (build hg19). Associated gene: target gene name from the UCSC database. The P-value of the Complete Response is derived from the Fisher's exact test (CR vs NR/SD/PD).

When a positive signature derived from the panel of the 7 or of the 32 CpG sites is established, the subject is considered that will completely respond to the therapy.

In a particular embodiment of the first aspect, the in vitro method allows determining the response of a subject to an autologous CAR T-cell therapy is a complete response, which means that no remission of the disease is observed after therapy.

As will be illustrated in the example below, the panel of 18 CpG sites, and thus the therein included panel of 6 cytosines (i.e., 6 CpG sites) particularized in a previous embodiment, as well as the panel of 32 cytosines in CpG sites, and thus the therein included of 7 cytosines (i.e., 7 CpG sites) particularized in a previous embodiment, were advantageously applicable independently of the B-cell malignancy the subject from which the sample was isolated was derived from. Thus, the one or more CpG sites, in particular these six or seven, and also the eighteen or thirty-two are reliable and applicable to CAR T-cells derived from isolated samples from subjects suffering any B-malignancy.

A “B-cell malignancy” (or B-cell lymphoma, as a synonymous term) is a cancer that forms in B cells, which grow out of control. B-cell lymphomas may be either indolent (slow-growing) or aggressive (fast-growing). In order to diagnose the presence of a B-cell malignancy, the doctor determines by several means if the number of B-cells in a sample of a patient is over a threshold. The skilled person in the art will know about the sampling and analysis for the diagnosis of a B-cell malignancy and for the sub-classification among the several existing subtypes. In a particular embodiment, the B-cell malignancy is selected from the group consisting of B-cell acute lymphoblastic leukemia (ALL), diffuse large B-cell lymphoma (DLBCL), non-Hodgkin lymphoma (NHL), primary mediastinal B-cell lymphoma (PMBCL); follicular lymphoma (FL); mantle cell lymphoma chronic lymphocytic leukemia (MCL), multiple myeloma, neuroblastoma, glioblastoma, and advanced gliomas. In a more particular embodiment, the B-cell malignancy is selected from B-cell acute lymphoblastic leukemia (ALL), and non-Hodgkin lymphoma (NHL).

The isolated sample of the subject comprising T-cells is to be understood as any tissue (e.g., tissue biopsy) or biofluid (e.g., peripheral blood) from which T-cells are directly obtained, or including cells, in particular mononuclear cells that can be derived to T-cells following the activation in the appropriated cell medium the skilled person will know. These obtained T-cells are the ones being transduced with the CAR that will recognize, once expressed in the cell membrane, tumour antigens.

In another particular embodiment of the first aspect, optionally in combination with any embodiments above or below, the isolated sample is selected from a biofluid including lymphocyte T cells. More in particular it is selected from blood, leukapheresis fluid comprising peripheral-blood mononuclear cells, and combinations thereof. In a particular embodiment, the isolated sample is leukapheresis fluid comprising peripheral-blood mononuclear cells that are activated in T-cell medium to obtain a population of T-cells that will be CAR-transduced.

In another particular embodiment of the first aspect the CAR is one targeting an antigen-associated tumor and selected from the group consisting of B-lymphocyte antigen CD19 (UNIPROT P15391, isoform 1 as canonical sequence, version 6 of sequence of 13 Nov. 2007, version 215 of UniprotKB database). Other CARs target more than one antigen-associated tumors (i.e. CD19, CDS, CD20). These multiple-targeting CARs are proteins (usually fusion proteins) that allow the targeting of multiple antigen-associated tumors.

In a more particular embodiment of the first aspect, the CAR is the B-lymphocyte antigen CD19. Thus, the CAR comprises an extracellular antigen-binding element that specifically binds to the B-lymphocyte antigen CD19, an extracellular and a transmembrane region. In an even more particular embodiment, the antigen-binding element is an anti-CD 19 scFv, even more preferably the mouse or human anti-CD 19 scFv. These antigen-binding elements are for example disclosed in the international patent application WO2015187528. In even a more particular embodiment, the CAR comprises anti-CD19 monoclonal antibody FMC63, or a fragment thereof, fused to the CD28 costimulatory domain and to the CD3 zeta chain. More in particular in this fusion protein, the scFv is derived from mAb clone FMC63 that binds human CD19 and it is generated by fusing the V_{L} and V_{H} regions via a “Whitlow” linker peptide; this scFV is then attached to modified human IgG₄ hinge and CH₂—CH₃ regions and fused to the CD28 (transmembrane and cytoplasmic) and CD3 zeta chain (cytoplasmic) domains.

Determination of methylation status can be carried out by several methods and techniques known by the skilled person in the art.

One of the most common includes the use of probes that are specific for the methylation sites. Thus, in another particular embodiment of the in vitro method of the first aspect, the methylation status of the one or more cytosines in the CpG sites is determined with a set of DNA oligonucleotides comprising one or more oligonucleotides that are complementary to a sequence comprising the cytosine of each CpG and producing a differential signal if the cytosine in determined positions is methylated or unmethylated. In a more particular embodiment, the differential signal is selected from fluorescence signal, chemiluminescence signal and combinations thereof. This signal is mainly the result of the emission of either fluorescence or chemiluminescence by a compound associated, in particular, covalently bonded, to the oligonucleotides complementary to the sequences to be detected.

Alternatively, in another embodiment, the methylation status of the one or more cytosines in the CpG sites is determined with a set of DNA oligonucleotides comprising one or more oligonucleotides that are complementary to a sequence comprising the methylated cytosine of each CpG site, and one or more oligonucleotides that are complementary to a sequence comprising the unmethylated cytosine of each CpG site.

In an example, the methylation status of any individual cytosines or a group of cytosines in the genome of a CAR T-cell (e.g., CD8 T cell) can be determined using standardised methodologies, including among others the Infinium MethylationEPIC Array (approximately 850,000 CpG sites) and the automated processing of arrays with a liquid handler (Illumina Infinium HD Methylation Assay Experienced User Card). Other examples include the Illumina® HumanMethylation 450 Bead Chip kit. Illumina® HumanMethylation 450 Bead Chip kit is a method based on highly multiplexed genotyping of bisulfite-converted genomic DNA. Upon treatment with bisulfite, unmethylated cytosine bases are converted to uracil, while methylated cytosine bases remain unchanged. These chemically-differentiated loci are interrogated using two site-specific probes (DNA oligonucleotides), one designed for the methylated locus and one designed for the unmethylated locus of a particular genomic region. The probes incorporate labelled ddNTP, which is subsequently stained with a fluorescent reagent. Level of methylation for the interrogated locus can be determined by calculating the ratio of the fluorescent signals from the methylated vs unmethylated sites. Other methodologies for the establishment of DNA methylation signatures are known for the expert and include Methylation-Specific PCR (MSP), ChIP-on-chip assays, Pyrosequencing of bisulfite treated DNA, and High Resolution Melt Analysis (HRM or HRMA), restriction landmark genomic scanning, COBRA, Ms-SNuPE, methylated DNA immunoprecipitation (MeDip), pyrosequencing of bisulfite treated DNA, molecular break light assay for DNA adenine methyltransferase activity, methyl sensitive Southern blotting, methyl CpG binding proteins, mass spectrometry, HPLC, and reduced representation bisulfite sequencing. In some embodiments methylation is detected at specific sites of DNA methylation using pyrosequencing after bisulfite treatment and optionally after amplification of the methylation sites. Pyrosequencing technology is a method of sequencing-by-synthesis in real time. In some embodiments, the DNA methylation is detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray. In specific embodiments, detection of DNA methylation can be performed by first converting the DNA to be analyzed so that the unmethylated cytosine is converted to uracil. In one embodiment, a chemical reagent that selectively modifies either the methylated or non-methylated form of CpG dinucleotide motifs may be used. Suitable chemical reagents include hydrazine and bisulphite ions and the like. For example, isolated DNA can be treated with sodium bisulfite (NaHS03) which converts unmethylated cytosine to uracil, while methylated cytosines are maintained. Without wishing to be bound by a theory, it is understood that sodium bisulfite reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonated group can be removed under alkaline conditions, resulting in the formation of uracil. The nucleotide conversion results in a change in the sequence of the original DNA. It is general knowledge that the resulting uracil has the base pairing behavior of thymine, which differs from cytosine base pairing behavior. To that end, uracil is recognized as a thymine by DNA polymerase. Therefore, after PCR or sequencing, the resultant product contains cytosine only at the position where 5-methylcytosine occurs in the starting template DNA. This makes the discrimination between unmethylated and methylated cytosine possible.

In another particular embodiment of the first aspect, the method for determining the response to an autologous CAR T-cell therapy further comprises isolating an identified candidate CAR T-cell or a population thereof, and optionally expanding the isolated candidate CAR T cell or population thereof to obtain an expanded candidate CAR T-cell or population thereof.

With this particular embodiment, expansion of the CAR T-cells is achieved, and the cells are prepared to be administered to the subject in need thereof. Skilled person will know the different methods for expanding these type of cells as well as the method of formulating them in approved pharmaceutically compositions comprising one or more carriers and excipients.

Inventors have also found out that with the addition of the analysis of other cytosines in CpG sites, important information regarding appearance of adverse effects associated with cell adoptive therapy could be provided. In particular, the determination of the appearance of the cytokine release syndrome (CRS); and/or the immune effector cell-associated neurotoxicity syndrome (ICANS).

Thus, in another particular embodiment of the in vitro method according to the first aspect, the method further comprises determining the methylation status of one or more CpG sites of the CAR T-cells obtained by transduction of the T cells in the isolated sample, the one or more CpG sites of CAR T cells selected from the group consisting of: cytosine at position 131058184 of human chromosome 2 (cg01311063); cytosine at position 36259383 of human chromosome 21 (cg00994804); cytosine at position 180614858 of human chromosome 5 (cg25606201); cytosine at position 18899483 of human chromosome 19 (cg26669806); cytosine at position 190448126 of human chromosome 1 (cg24365464); cytosine at position 201123894 of human chromosome 1 (cg09554300); cytosine at position 45505849 of human chromosome 3 (cg14416782); cytosine at position 2075777 of human chromosome 8 (cg21847720); cytosine at position 218340518 of human chromosome 2 (cg14538944); cytosine at position 85637673 of human chromosome 2 (cg19627006); cytosine at position 10415636 of human chromosome 6 (cg22836400); cytosine at position 29990921 of human chromosome 14 (cg20017856); cytosine at position 100879199 of human chromosome 15 (cg07199183); cytosine at position 105648138 of human chromosome 10 (cg11005552); cytosine at position 637813 of human chromosome 8 (cg14755254); cytosine at position 110721138 of human chromosome 6 (cg19196401); and cytosine at position 130516192 of human chromosome 12 (cg15612205), all cytosine positions in human chromosomes according to the chromosome map and sequence entries of database UCSC Genome Browser on Human February 2009, GRCh37/hg19 assembly of the University of California Santa Cruz (UCSC).

In a more particular embodiment of the in vitro method of the first aspect, it comprises further determining the methylation status of the one or more CpG sites of CAR T cells selected from the group consisting of: cytosine at position 131058184 of human chromosome 2 (cg01311063); cytosine at position 36259383 of human chromosome 21 (cg00994804); cytosine at position 180614858 of human chromosome 5 (cg25606201); cytosine at position 18899483 of human chromosome 19 (cg26669806); cytosine at position 190448126 of human chromosome 1 (cg24365464) cytosine at position 2075777 of human chromosome 8 (cg21847720); cytosine at position 218340518 of human chromosome 2 (cg14538944); and cytosine at position 10415636 of human chromosome 6, (cg22836400).

These additional eight cytosines in CpG sites give, in particular very accurate information regarding the appearance of CRS. In a particular embodiment all eight are determined in combination with the one or more previously listed and giving information about complete response to CAR T-cell therapy. Thus, the method of the first aspect, comprises, in another yet more particular embodiment, determining the methylation status of the following CpG sites of CART cells: cytosine at position 131058184 of human chromosome 2 (cg01311063); cytosine at position 36259383 of human chromosome 21 (cg00994804); cytosine at position 180614858 of human chromosome 5 (cg25606201); cytosine at position 18899483 of human chromosome 19 (cg26669806); cytosine at position 190448126 of human chromosome 1 (cg24365464); cytosine at position 2075777 of human chromosome 8 (cg21847720); cytosine at position 218340518 of human chromosome 2 (cg14538944); and cytosine at position 10415636 of human chromosome 6, (cg22836400).

In another particular embodiment, the methylation status is determined in one, two, three, four, five, six or seven cytosines providing the accurate information of appearance of CRS.

In another alternative particular embodiment of the in vitro method according to the first aspect, the method further comprises determining the methylation status of one or more CpG sites of the CAR T-cells obtained by transduction of the T cells in the isolated sample, the one or more CpG sites of CAR T cells selected from the group consisting of: cytosine at position 201123894 of human chromosome 1 (cg09554300); cytosine at position 45505849 of human chromosome 3 (cg14416782); cytosine at position 2075777 of human chromosome 8 (cg21847720); cytosine at position 218340518 of human chromosome 2 (cg14538944); cytosine at position 85637673 of human chromosome 2 (cg19627006); cytosine at position 10415636 of human chromosome 6 (cg22836400); cytosine at position 29990921 of human chromosome 14 (cg20017856); cytosine at position 100879199 of human chromosome 15 (cg07199183); cytosine at position 105648138 of human chromosome 10 (cg11005552); cytosine at position 637813 of human chromosome 8 (cg14755254); cytosine at position 110721138 of human chromosome 6 (cg19196401); and cytosine at position 130516192 of human chromosome 12 (cg15612205), all cytosine positions in human chromosomes according to the chromosome map and sequence entries of database UCSC Genome Browser on Human February 2009, GRCh37/hg19 assembly of the University of California Santa Cruz (UCSC).

These additional twelve cytosines in CpG sites also give, in particular, information regarding the appearance of CRS. In a particular embodiment all twelve are determined in combination with the one or more previously listed and giving information about complete response to CAR T-cell therapy. In another particular embodiment, the methylation status is determined in one, two, three, four, five six, seven, eight, nine, ten, or eleven of these twelve cytosines providing the information of appearance of CRS.

Indeed, the appearance of CRS due to the autologous CAR T-cell therapy is, effectively, determined with the analysis of one or more CpG sites different from the ones giving the information of the complete response and, in particular of high EFS and OS. Thus, it is also herewith disclosed an in vitro method for predicting the appearance of CRS due to an autologous CAR T-cell therapy, this method comprising:
(a) determining the methylation status of one or more cytosines in CpG sites of CAR T-cells, said CAR T-cells obtained from an isolated sample of the subject comprising T-cells that, once isolated, have been transduced with the CAR, the one or more cytosines in CpG sites of CAR T-cells selected from the group consisting of: cytosine at position 131058184 of human chromosome 2 (cg01311063); cytosine at position 36259383 of human chromosome 21 (cg00994804); cytosine at position 180614858 of human chromosome 5 (cg25606201); cytosine at position 18899483 of human chromosome 19 (cg26669806); cytosine at position 190448126 of human chromosome 1 (cg24365464); cytosine at position 201123894 of human chromosome 1 (cg09554300); cytosine at position 45505849 of human chromosome 3 (cg14416782); cytosine at position 2075777 of human chromosome 8 (cg21847720); cytosine at position 218340518 of human chromosome 2 (cg14538944); cytosine at position 85637673 of human chromosome 2 (cg19627006); cytosine at position 10415636 of human chromosome 6 (cg22836400); cytosine at position 29990921 of human chromosome 14 (cg20017856); cytosine at position 100879199 of human chromosome 15 (cg07199183); cytosine at position 105648138 of human chromosome 10 (cg11005552); cytosine at position 637813 of human chromosome 8 (cg14755254); cytosine at position 110721138 of human chromosome 6 (cg19196401); and cytosine at position 130516192 of human chromosome 12 (cg15612205), all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of appearance of CRS in response to autologous CAR T-cell therapy, and determining the appearance of CRS in the subject if the methylation status is of the one or more CpG sites equal to said reference value or within the range of reference values.

In a particular example of the method of predicting CRS appearance the methylation status of all the CpG sites is determined (i.e., 17 CpG sites). In a more particular example, the methylation status of the one or more CpG sites of CAR T cells selected from the group consisting of: cytosine at position 131058184 of human chromosome 2 (cg01311063); cytosine at position 36259383 of human chromosome 21 (cg00994804); cytosine at position 180614858 of human chromosome 5 (cg25606201); cytosine at position 18899483 of human chromosome 19 (cg26669806); cytosine at position 190448126 of human chromosome 1 (cg24365464); cytosine at position 2075777 of human chromosome 8 (cg21847720); cytosine at position 218340518 of human chromosome 2 (cg14538944); and cytosine at position 10415636 of human chromosome 6, (cg22836400). In a more particular example, the methylation of the eight CpG sites is determined. In another example only one, two, three, four, five, six or seven of these eight CpG sites are determined in a method of predicting CRS appearance.

It is also herewith disclosed an alternative example of the in vitro method for predicting the appearance of CRS due to an autologous CAR T-cell therapy, this method comprising: (a) determining the methylation status of one or more cytosines in CpG sites of CAR T-cells, said CAR T-cells obtained from an isolated sample of the subject comprising T-cells that, once isolated, have been transduced with the CAR, the one or more cytosines in CpG sites of CAR T-cells selected from the group consisting of: cytosine al position 201123894 of human chromosome 1 (cg09554300); cytosine at position 45505849 of human chromosome 3 (cg14416782); cytosine at position 2075777 of human chromosome 8 (cg21847720); cytosine at position 218340518 of human chromosome 2 (cg14538944); cytosine at position 85637673 of human chromosome 2 (cg19627006); cytosine at position 10415636 of human chromosome 6 (cg22836400); cytosine at position 29990921 of human chromosome 14 (cg20017856); cytosine at position 100879199 of human chromosome 15 (cg07199183); cytosine at position 105648138 of human chromosome 10 (cg11005552); cytosine at position 637813 of human chromosome 8 (cg14755254); cytosine at position 110721138 of human chromosome 6 (cg19196401); and cytosine at position 130516192 of human chromosome 12 (cg15612205), all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of appearance of CRS in response to autologous CAR T-cell therapy, and determining the appearance of CRS in the subject if the methylation status is of the one or more CpG sites equal to said reference value or within the range of reference values.

In a particular example of the method of predicting CRS appearance the methylation status of the twelve CpG sites is determined to predict the appearance of CRS. In another particular embodiment, the methylation status is determined in one, two, three, four, five six, seven, eight, nine, ten, or eleven of these twelve cytosines providing the information of appearance of CRS.

In yet another particular embodiment, of the in vitro method according to the first aspect (i.e., a method for predicting response to CAR T-cell therapy), the method further comprises determining the methylation status of one or more CpG sites of the CAR T cells obtained by transduction of the T-cells in the isolated sample, the one or more CpG sites of CAR T-cells selected from the group consisting of: cytosine at position 131058184 of human chromosome 2 (cg01311063); cytosine at position 102242535 of human chromosome 10 (cg26195366); cytosine at position 23015936 of human chromosome 20 (cg22534145); cytosine at position 134571377 of human chromosome 10 (cg27196695); cytosine at position 65294635 of human chromosome 8 (cg27272679); cytosine at position 100879199 of human chromosome 15 (cg07199183); cytosine at position 32353565 of human chromosome 11 (cg09992216); cytosine at position 124132919 of human chromosome 9 (cg14215970); cytosine at position 42299379 of human chromosome 19 (cg24267358); cytosine at position 180614858 of human chromosome 5 (cg25606201); cytosine at position 134457731 of human chromosome 10 (cg25268100); cytosine at position 22634199 of human chromosome 10 (cg12610471); cytosine at position 686450 of human chromosome 17 (cg12197459); cytosine at position 90081872 of human chromosome 14 (cg12504912); cytosine at position 127568850 of human chromosome 8 (cg21390512); cytosine at position 94057587 of human chromosome 1 (cg02978297); cytosine at position 134149184 of human chromosome 10 (cg14683065), cytosine at position 17109239 of human chromosome 17 (cg01412970); cytosine at position 36258423 of human chromosome 21 (cg01664727); cytosine at position 132481826 of human chromosome 2 (cg14161159), cytosine at position 104535854 of human chromosome 10 (cg15227982); and cytosine at position 190448126 of human chromosome 1 (cg24365464), all cytosine positions in human chromosomes according to the chromosome map and sequence entries of database UCSC Genome Browser on Human February 2009, GRCh37/hg19 assembly of the University of California Santa Cruz (UCSC).

In a more particular embodiment of the in vitro method of the first aspect, it comprises further determining the methylation status of the one or more CpG sites of CAR T cells selected from the group consisting of: cytosine at position 131058184 of human chromosome 2 (cg01311063); cytosine at position 102242535 of human chromosome 10 (cg26195366); cytosine at position 23015936 of human chromosome 20 (cg22534145); cytosine at position 134571377 of human chromosome 10 (cg27196695); and cytosine at position 65294635 of human chromosome 8 (cg27272679). These in particular additional five cytosines in CpG sites give, highly accurate information regarding the appearance of ICANS. In a particular embodiment all five are determined in combination with the one or more previously listed and giving information about complete response to CAR T-cell therapy. In another particular embodiment, the methylation status is determined in only one, two, three, or four, of these five cytosines providing the information of appearance of ICANS.

In yet another particular embodiment of the in vitro method according to the first aspect (i.e., a method for predicting response to CAR T-cell therapy), the method further comprises determining the methylation status of one or more CpG sites of the CAR T cells obtained by transduction of the T-cells in the isolated sample, the one or more CpG sites of CAR T-cells selected from the group consisting of: cytosine at position 65294635 of human chromosome 8 (cg27272679); cytosine at position 100879199 of human chromosome 15 (cg07199183); cytosine at position 32353565 of human chromosome 11 (cg09992216); cytosine at position 124132919 of human chromosome 9 (cg14215970); cytosine at position 42299379 of human chromosome 19 (cg24267358); cytosine at position 180614858 of human chromosome 5 (cg25606201); cytosine at position 134457731 of human chromosome 10 (cg25268100); cytosine at position 22634199 of human chromosome 10 (cg12610471); cytosine at position 686450 of human chromosome 17 (cg12197459); cytosine at position 90081872 of human chromosome 14 (cg12504912); cytosine at position 127568850 of human chromosome 8 (cg21390512); cytosine at position 94057587 of human chromosome 1 (cg02978297); cytosine at position 134149184 of human chromosome 10 (cg14683065), cytosine at position 17109239 of human chromosome 17 (cg01412970); cytosine at position 36258423 of human chromosome 21 (cg01664727); cytosine at position 132481826 of human chromosome 2 (cg14161159), cytosine at position 104535854 of human chromosome 10 (cg15227982); and cytosine at position 190448126 of human chromosome 1 (cg24365464), all cytosine positions in human chromosomes according to the chromosome map and sequence entries of database UCSC Genome Browser on Human February 2009, GRCh37/hg19 assembly of the University of California Santa Cruz (UCSC).

These additional eighteen cytosines in CpG sites also give, in particular, information regarding the appearance of ICANS. In a particular embodiment all eighteen are determined in combination with the one or more previously listed and giving information about complete response to CAR T-cell therapy. In another particular embodiment, the methylation status is determined in one, two, three, four, five six, seven, eight, nine, ten, eleven, twelve, thirteen, fourteen, fifteen, sixteen or seventeen of these eighteen cytosines providing the information of appearance of ICANS.

Indeed, the appearance of ICANS due to the autologous CAR T-cell therapy is, effectively, determined with the analysis of one or more CpG sites different from the ones giving the information of the complete response and, in particular of high EFS and OS. Thus, it is also herewith disclosed an in vitro method for predicting the appearance of ICANS due to an autologous CAR T-cell therapy, this method comprising:
(a) determining the methylation status of one or more cytosines in CpG sites of CAR T-cells, said CAR T-cells obtained from an isolated sample of the subject comprising T-cells that, once isolated, have been transduced with the CAR, the one or more cytosines in CpG sites of CAR T-cells selected from the group consisting of: cytosine at position 131058184 of human chromosome 2 (cg01311063); cytosine at position 102242535 of human chromosome 10 (cg26195366); cytosine at position 23015936 of human chromosome 20 (cg22534145); cytosine at position 134571377 of human chromosome 10 (cg27196695); cytosine at position 65294635 of human chromosome 8 (cg27272679); cytosine at position 100879199 of human chromosome 15 (cg07199183); cytosine at position 32353565 of human chromosome 11 (cg09992216); cytosine at position 124132919 of human chromosome 9 (cg14215970); cytosine at position 42299379 of human chromosome 19 (cg24267358); cytosine at position 180614858 of human chromosome 5 (cg25606201); cytosine at position 134457731 of human chromosome 10 (cg25268100); cytosine at position 22634199 of human chromosome 10 (cg12610471); cytosine at position 686450 of human chromosome 17 (cg12197459); cytosine at position 90081872 of human chromosome 14 (cg12504912); cytosine at position 127568850 of human chromosome 8 (cg21390512); cytosine at position 94057587 of human chromosome 1 (cg02978297); cytosine at position 134149184 of human chromosome 10 (cg14683065), cytosine at position 17109239 of human chromosome 17 (cg01412970); cytosine at position 36258423 of human chromosome 21 (cg01664727); cytosine at position 132481826 of human chromosome 2 (cg14161159), cytosine at position 104535854 of human chromosome 10 (cg15227982); and cytosine at position 190448126 of human chromosome 1 (cg24365464), all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of appearance of ICANS in response to autologous CAR T-cell therapy and determining the appearance of ICANS in the subject if the methylation status is of the one or more CpG sites equal to said reference value or within the range of reference values.

In a particular example of the method of predicting ICANS appearance the methylation status of all the CpG sites is determined (i.e., 22 CpG sites). In a more particular example, the methylation status of the one or more CpG sites of CAR T cells selected from the group consisting of: cytosine at position 131058184 of human chromosome 2 (cg01311063); cytosine at position 102242535 of human chromosome 10 (cg26195366); cytosine at position 23015936 of human chromosome 20 (cg22534145); cytosine at position 134571377 of human chromosome 10 (cg27196695); and cytosine at position 65294635 of human chromosome 8 (cg27272679). In a more particular example, the methylation of the five CpG sites is determined. In another example only one, two, three, or four of these five CpG sites are determined in a method of predicting ICANS appearance.

It is also herewith disclosed an alternative example of the in vitro method for predicting the appearance of ICANS due to an autologous CAR T-cell therapy, this method comprising: (a) determining the methylation status of one or more cytosines in CpG sites of CAR T-cells, said CAR T-cells obtained from an isolated sample of the subject comprising T-cells that, once isolated, have been transduced with the CAR, the one or more cytosines in CpG sites of CAR T-cells selected from the group consisting of; cytosine at position 65294635 of human chromosome 8 (cg27272679); cytosine at position 100879199 of human chromosome 15 (cg07199183); cytosine at position 32353565 of human chromosome 11 (cg09992216); cytosine at position 124132919 of human chromosome 9 (cg14215970); cytosine at position 42299379 of human chromosome 19 (cg24267358); cytosine at position 180614858 of human chromosome 5 (cg25606201); cytosine at position 134457731 of human chromosome 10 (cg25268100); cytosine at position 22634199 of human chromosome 10 (cg12610471); cytosine at position 686450 of human chromosome 17 (cg12197459); cytosine at position 90081872 of human chromosome 14 (cg12504912); cytosine at position 127568850 of human chromosome 8 (cg21390512); cytosine at position 94057587 of human chromosome 1 (cg02978297); cytosine at position 134149184 of human chromosome 10 (cg14683065), cytosine at position 17109239 of human chromosome 17 (cg01412970); cytosine at position 36258423 of human chromosome 21 (cg01664727); cytosine at position 132481826 of human chromosome 2 (cg14161159), cytosine at position 104535854 of human chromosome 10 (cg15227982); and cytosine at position 190448126 of human chromosome 1 (cg24365464), all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of appearance of ICANS in response to autologous CAR T-cell therapy and determining the appearance of ICANS in the subject if the methylation status is of the one or more CpG sites equal to said reference value or within the range of reference values.

In particular the methylation status of the eighteen CpG sites is determined to predict the appearance of ICANS. In another particular embodiment, the methylation status is determined in the methylation status is determined in one, two, three, four, five six, seven, eight, nine, ten, eleven, twelve, thirteen, fourteen, fifteen, sixteen or seventeen of these eighteen cytosines providing the information of appearance of ICANS.

Another aspect of the invention is a method of deciding and/or recommending whether to initiate a CAR T-cell therapy for a subject suffering from B-cell malignancies, which method comprises carrying out the in vitro method as defined in the first aspect; and wherein if the subject is determined to respond to CAR T-cell therapy, then this therapy is decided and/or recommended.

All particular embodiments of the first aspect apply to this second aspect. Thus, the sample type and processing thereof, the addition of one or more cytosines to be determined, the type of CAR transduced in the cells, the B-cell malignancies, as well as any other particularities of the first aspect.

In a third aspect, the invention relates to a method of modulating methylation profiles in CpG sites of CAR T-cells, the method comprising the step of first carrying out the method as defined in the first aspect; and further modulating methylation profiles related with the differentiation and/or efficacy of therapy, said modulating carried out by means of methods as disclosed by previous authors and known by the skilled person in the art.

In a particular embodiment of this aspect, the modulating of this efficacy is carried out by means of DNA hypomethylating agents approved for clinical use in hematological malignancies, more in particular selected from decitabine or vidaza; histone deacetylase inhibitors, more in particular vorinostat; and histone methyltransferase inhibitors, more in particular EZH2 inhibitor tazverik, approved for use for treating subtypes of lymphomas and sarcomas.

The invention also relates to an in vitro in vitro method of identifying, in a sample taken from a human subject, the presence or absence of methylation in one or more CpG sites of CAR T cells selected from the group consisting of: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); cytosine at position 22634199 of human chromosome 10 (cg12610471): cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177) and cytosine at position 28725934 of human chromosome 8 (cg08544307), all cytosine positions in human chromosomes according to the chromosome map and sequence entries of database UCSC Genome Browser on Human February 2009, GRCh37/hg19 assembly of the University of California Santa Cruz (UCSC).

In a particular example of this method of identifying, in a sample taken from a human subject, the method comprises determining the methylation in all the following six cytosines: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); cytosine at position 22634199 of human chromosome 10 (cg12610471).

Another particular example of this method of identifying, in a sample taken from a human subject, the presence or absence of methylation in one or more CpG sites of CAR T cells, the in vitro method further comprises determining the methylation in one or more CpG sites of CAR T-cells selected from the group consisting of: cytosine at position 95139986 of human chromosome 10 (cg10039734); cytosine at position 127612751 of human chromosome 6 (cg25571136); cytosine at position 131058184 of human chromosome 2 (cg01311063); cytosine at position 90081872 of human chromosome 14 (cg12504912); cytosine at position 123944014 of human chromosome 12 (cg10236435); cytosine at position 134457731 of human chromosome 10 (cg25268100); cytosine at position 46993515 of human chromosome 10 (cg25995980); cytosine at position 209809 of human chromosome 6 (cg15253304); cytosine at position 122144477 of human chromosome 2 (cg17511575); cytosine at position 6643814 of human chromosome 6 (cg09367268); cytosine at position 60877850 of human chromosome 18 (cg11416737); and cytosine at position 42299379 of human chromosome 19 (cg24267358), all cytosine positions in human chromosomes according to the chromosome map and sequence entries of database UCSC Genome Browser on Human February 2009, GRCh37/hg19 assembly of the University of California Santa Cruz (UCSC).

In another more particular example, the method of identifying the presence or absence of methylation, comprises determining the methylation in all the following eighteen cytosines: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); cytosine at position 22634199 of human chromosome 10 (cg12610471); 95139986 of human chromosome 10 (cg10039734); cytosine at position 127612751 of human chromosome 6 (cg25571136); cytosine at position 131058184 of human chromosome 2 (cg01311063); cytosine at position 90081872 of human chromosome 14 (cg12504912); cytosine at position 123944014 of human chromosome 12 (cg10236435); cytosine at position 134457731 of human chromosome 10 (cg25268100); cytosine at position 46993515 of human chromosome 10 (cg25995980); cytosine at position 209809 of human chromosome 6 (cg15253304); cytosine at position 122144477 of human chromosome 2 (cg17511575); cytosine at position 6643814 of human chromosome 6 (cg09367268); cytosine at position 60877850 of human chromosome 18 (cg11416737); and cytosine at position 42299379 of human chromosome 19 (cg24267358).

In an alternative particular example of this method of identifying, in a sample taken from a human subject, the presence or absence of methylation in one or more CpG sites of CAR T cells selected from the group consisting of:
cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177) cytosine at position 28725934 of human chromosome 8 (cg08544307), all cytosine positions in human chromosomes according to the chromosome map and sequence entries of database UCSC Genome Browser on Human February 2009, GRCh37/hg19 assembly of the University of California Santa Cruz (UCSC), and wherein the presence or absence of methylation is performed by genotype methods.

In a particular example of this method of identifying, in a sample taken from a human subject, the method comprises determining the methylation in all the following seven cytosines: cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); and cytosine at position 79780164 of human chromosome 6 (cg13554177).

Another particular example of this method of identifying, in a sample taken from a human subject, the presence or absence of methylation in one or more CpG sites of CAR T cells, the in vitro method further comprises determining the methylation in one or more of: cytosine at position 134457731 of human chromosome 10 (cg25268100); cytosine at position 127612751 of human chromosome 6 (cg25571136), cytosine at position 6643814 of human chromosome 6 (cg09367268); cytosine at position 42299379 of human chromosome 19 (cg24267358); cytosine at position 32353565 of human chromosome 11 (cg09992216); cytosine at position 234087867 of human chromosome 1 (cg25534076); cytosine at position 105907265 of human chromosome 6 (cg04267686); cytosine at position 22634199 of human chromosome 10 (cg12610471); cytosine at position 95870440 of human chromosome 15 (cg18739950); cytosine at position 104470719 of human chromosome 10 (cg12700402); cytosine at position 43253559 of human chromosome 22 (cg01029450); cytosine at position 122144477 of human chromosome 2 (cg17511575); cytosine at position 131166906 of human chromosome 12 (cg26098972); cytosine at position 68481342 of human chromosome 16 (cg05948940); cytosine at position 100879199 of human chromosome 15 (cg07199183); cytosine at position 95139986 of human chromosome 10 (cg10039734); cytosine at position 183063459 of human chromosome 4 (cg19759671); cytosine at position 180614858 of human chromosome 5 (cg25606201); cytosine at position 134571377 of human chromosome 10 (cg27196695); cytosine at position 3600764 of human chromosome 12 (cg11596580); cytosine at position 90081872 of human chromosome 14 (cg12504912); cytosine at position 133000178 of human chromosome 12 (cg09698465); cytosine at position 46993515 of human chromosome 10 (cg25995980); cytosine at position 19229767 of human chromosome 9 (cg13469590); and cytosine at position 24229300 of human chromosome 1 (cg24452347), all cytosine positions in human chromosomes according to the chromosome map and sequence entries of database UCSC Genome Browser on Human February 2009, GRCh37/hg19 assembly of the University of California Santa Cruz (UCSC).

In another more particular example, the method of identifying the presence or absence of methylation, comprises determining the methylation in all the following thirty-two cytosines: cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177); cytosine at position 134457731 of human chromosome 10 (cg25268100); cytosine at position 127612751 of human chromosome 6 (cg25571136), cytosine at position 6643814 of human chromosome 6 (cg09367268); cytosine at position 42299379 of human chromosome 19 (cg24267358); cytosine at position 32353565 of human chromosome 11 (cg09992216); cytosine at position 234087867 of human chromosome 1 (cg25534076); cytosine at position 105907265 of human chromosome 6 (cg04267686); cytosine at position 22634199 of human chromosome 10 (cg12610471); cytosine at position 95870440 of human chromosome 15 (cg18739950); cytosine at position 104470719 of human chromosome 10 (cg12700402); cytosine at position 43253559 of human chromosome 22 (cg01029450); cytosine at position 122144477 of human chromosome 2 (cg17511575); cytosine at position 131166906 of human chromosome 12 (cg26098972); cytosine at position 68481342 of human chromosome 16 (cg05948940); cytosine at position 100879199 of human chromosome 15 (cg07199183); cytosine at position 95139986 of human chromosome 10 (cg10039734); cytosine at position 183063459 of human chromosome 4 (cg19759671); cytosine at position 180614858 of human chromosome 5 (cg25606201); cytosine at position 134571377 of human chromosome 10 (cg27196695); cytosine at position 3600764 of human chromosome 12 (cg11596580); cytosine at position 90081872 of human chromosome 14 (cg12504912); cytosine at position 133000178 of human chromosome 12 (cg09698465); cytosine at position 46993515 of human chromosome 10 (cg25995980); cytosine at position 19229767 of human chromosome 9 (cg13469590); and cytosine at position 24229300 of human chromosome 1 (cg24452347).

Herewith disclosed is also a method of treating a subject suffering from B-cell malignancies, the method comprising administering to said subject an autologous CAR T-cell therapy, and wherein the method also comprises:
(a) a step of first determining if the subject will respond to said autologous CAR T-cell therapy by determining the methylation status of one or more CpG sites of CAR T-cells, said CAR T-cells obtained from an isolated sample of the subject comprising T-cells that, once isolated, have been transduced with the CAR; (b) a step of comparing the methylation status of the one or more CpG sites with a reference value or range of values of response to CAR T-cell therapy, and determining the subject will respond to CAR T-cell therapy if the methylation status of the one or more CpG sites is equal to said reference value or within the range of reference values; and (c) treating the subject with the autologous CAR T-cell therapy if determined as respondent to the said therapy.

Thus, if the subject is determined as respondent to the autologous therapy, in a particular embodiment, an isolated candidate CAR T-cell or population thereof, or an expanded candidate CAR T-cell or population thereof is administered to a subject in need thereof.

In a particular embodiment, the isolated candidate CAR T-cell or population thereof, or an expanded candidate CAR T-cell or population thereof, prior to the infusion into the subject, is submitted to an ex vivo intervention for modulating its efficacy by contacting the CAR T-cells with an agent selected from a DNA hypomethylating agents approved for clinical use in hematological malignancies, more in particular selected from decitabine or vidaza; an histone deacetylase inhibitor, more in particular vorinostat; and an histone methyltransferase inhibitors, more in particular EZH2 inhibitor tazverik.

In a particular example of this method of treating, are determined the one or more CpG sites of CAR T-cells selected from the group consisting of: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); cytosine at position 22634199 of human chromosome 10 (cg12610471), cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177) and cytosine at position 28725934 of human chromosome 8 (cg08544307), all cytosine positions in human chromosomes according to the chromosome map and sequence entries of database UCSC Genome Browser on Human February 2009, GRCh37/hg19 assembly of the University of California Santa Cruz (UCSC).

In another particular example the methylation of one, two, three, four, five, six, seven, eight, nine, ten, eleven or the twelve cytosines is determined. In yet another example, the methylation status of the following six cytosines is determined: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); and cytosine at position 22634199 of human chromosome 10 (cg12610471).

In an alternative particular example of this method of treating, are determined the one or more CpG sites of CAR T-cells selected from the group consisting of: cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177) and cytosine at position 28725934 of human chromosome 8 (cg08544307), all cytosine positions in human chromosomes according to the chromosome map and sequence entries of database UCSC Genome Browser on Human February 2009, GRCh37/hg19 assembly of the University of California Santa Cruz (UCSC). In another particular example the methylation of one, two, three, four, five, six, seven or the eight cytosines is determined. In yet another example, the methylation status of the following seven cytosines is determined: cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); and cytosine at position 79780164 of human chromosome 6 (cg13554177).

Particular sample types isolated from the subject and from which T-cells can be obtained, particular combinations with additional methylation status of one or more cytosines, and particular B-cell malignancies, all indicated in particular embodiments of the first and second aspects, do also apply to this method of treating of a subject suffering from B-cell malignancies.

Present invention also comprises as another aspect the use of means comprising DNA oligonucleotides suitable for determining DNA methylation status of one or more CpG site cytosines, for predicting the response of a subject to chimeric antigen receptor T cell (CAR T cell) therapy, in any of the methods of the first and second aspect.

These means are, in a particular embodiment, DNA oligonucleotides that are complementary to a sequence comprising the cytosine of each CpG and producing a differential signal if the cytosine in a determined position is methylated or unmethylated. In a more particular embodiment, the differential signal is selected from fluorescence signal, chemiluminescence signal and combinations thereof. This signal is mainly the result of the emission of either fluorescence or chemiluminescence by a compound associated, in particular, covalently bonded, to the oligonucleotides complementary to the sequences to be detected. Alternatively, in another embodiment, the means comprise one or more DNA oligonucleotides that are complementary to a sequence comprising the methylated cytosine of each CpG site, and one or more DNA oligonucleotides that are complementary to a sequence comprising the unmethylated cytosine of each CpG site.

In another particular embodiment of the use of these means, the DNA oligonucleotides are provided together with other reagents, such as buffers, fluorescent or chemiluminescent labels, and instructions to use them in the determination of the methylation status of the one or more cytosines in de CpG sites of interest. Thus, in another particular embodiment, all these means form part of a kit comprising, the one or more reagent means for determining DNA methylation status of the one or more CpG site cytosines; and instructions for determining the methylation status.

Indeed, herewith proposed are also kits comprising reagent means for determining the DNA methylation status of one or more CpG site cytosines selected from: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); cytosine at position 22634199 of human chromosome 10 (cg12610471), cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177) and cytosine at position 28725934 of human chromosome 8 (cg08544307); and instructions for the use of the means for determining the DNA methylation status.

In a particular embodiment, the kits comprise reagent means for determining the DNA methylation status of one or more CpG site cytosines selected from: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); cytosine at position 22634199 of human chromosome 10 (cg12610471).

In another particular and alternative embodiment of the kits, they comprise reagent means for determining the DNA methylation status of one or more CpG site cytosines selected from: cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); and cytosine at position 79780164 of human chromosome 6 (cg13554177); and instructions for the use of the means for determining the DNA methylation status.

The term “kit”, as used herein, refers to a product containing the different reagents (or reagent means) necessary for carrying out the methods of the invention packed so as to allow their transport and storage. Materials suitable for packing the components of the kit include crystal, plastic (e.g. polyethylene, polypropylene, polycarbonate), bottles, vials, paper, or envelopes.

In a particular embodiment, the instructions in the kit are for the simultaneous, sequential or separate use of the different components which are in the kit. Said instructions can be in the form of printed material or in the form of an electronic support capable of storing instructions susceptible of being read or understood, such as, for example, electronic storage media (e.g. magnetic disks, tapes), or optical media (e.g. CD-ROM, DVD), or audio materials. Additionally, or alternatively, the media can contain internet addresses that provide said instructions.

In a preferred embodiment, the reagent means for determining the methylation status of the one or more cytosines in CpG sites comprise at least 10%, at least 20%, at least 30%, at least 40%, at least 50%, at least 60%, at least 70%, at least 80%, at least 90% or at least 100% of the total amount of reagents for determining the methylation status forming the kit. These kits are, thus, simplified kits including mainly the reagent means for determining the methylation status of the indicated cytosines in CpG sites.

In a particular example of the kit, it comprises the means for determining the methylation status of the six cytosines: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); and cytosine at position 22634199 of human chromosome 10 (cg12610471). In another more particular example, the means comprised in the kit consist of the means for determining only the methylation status of the seven cytosines in CpG sites.

In a particular example of the kit, it comprises the means for determining the methylation status of the seven cytosines in CpG sites: cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); and cytosine at position 79780164 of human chromosome 6 (cg13554177. In another more particular example, the means comprised in the kit consist of the means for determining only the methylation status of the seven cytosines in CpG sites.

In a particular embodiment of the kit, it comprises the means for determining the methylation status of the following eighteen cytosines in CpG sites: cytosine at position 86332162 of human chromosome 2 (cg12260379), cytosine at position 188676237 of human chromosome 1 (cg12012941); cytosine at position 105907265 of human chromosome 6 (cg04267686); cytosine at position 234087867 of human chromosome 1 (cg25534076); cytosine at position 32353565 of human chromosome 11 (cg09992216); cytosine at position 22634199 of human chromosome 10 (cg12610471); 95139986 of human chromosome 10 (cg10039734); cytosine at position 127612751 of human chromosome 6 (cg25571136); cytosine at position 131058184 of human chromosome 2 (cg01311063); cytosine at position 90081872 of human chromosome 14 (cg12504912); cytosine at position 123944014 of human chromosome 12 (cg10236435); cytosine at position 134457731 of human chromosome 10 (cg25268100); cytosine at position 46993515 of human chromosome 10 (cg25995980); cytosine at position 209809 of human chromosome 6 (cg15253304); cytosine at position 122144477 of human chromosome 2 (cg17511575); cytosine at position 6643814 of human chromosome 6 (cg09367268); cytosine at position 60877850 of human chromosome 18 (cg11416737); and cytosine at position 42299379 of human chromosome 19 (cg24267358).

In a particular embodiment of the kit, it comprises the means for determining the methylation status of the following thirty-two cytosines in CpG sites:
thirty-two cytosines: cytosine at position 86332162 of human chromosome 2 (cg12260379), cytosine at position 188676237 of human chromosome 1 (cg12012941); cytosine at position 45028225 of human chromosome 2 (cg03593578); cytosine at position 220414164 of human chromosome 1 (cg04458195); cytosine at position 209809 of human chromosome 6 (cg15253304); cytosine at position 62905816 of human chromosome 1 (cg22171055); cytosine at position 79780164 of human chromosome 6 (cg13554177); cytosine at position 134457731 of human chromosome 10 (cg25268100); cytosine at position 127612751 of human chromosome 6 (cg25571136), cytosine at position 6643814 of human chromosome 6 (cg09367268); cytosine at position 42299379 of human chromosome 19 (cg24267358); cytosine at position 32353565 of human chromosome 11 (cg09992216); cytosine at position 234087867 of human chromosome 1 (cg25534076); cytosine at position 105907265 of human chromosome 6 (cg04267686); cytosine at position 22634199 of human chromosome 10 (cg12610471); cytosine at position 95870440 of human chromosome 15 (cg18739950); cytosine at position 104470719 of human chromosome 10 (cg12700402); cytosine at position 43253559 of human chromosome 22 (cg01029450); cytosine at position 122144477 of human chromosome 2 (cg17511575); cytosine at position 131166906 of human chromosome 12 (cg26098972); cytosine at position 68481342 of human chromosome 16 (cg05948940); cytosine at position 100879199 of human chromosome 15 (cg07199183); cytosine at position 95139986 of human chromosome 10 (cg10039734); cytosine at position 183063459 of human chromosome 4 (cg19759671); cytosine at position 180614858 of human chromosome 5 (cg25606201); cytosine at position 134571377 of human chromosome 10 (cg27196695); cytosine at position 3600764 of human chromosome 12 (cg11596580); cytosine at position 90081872 of human chromosome 14 (cg12504912); cytosine at position 133000178 of human chromosome 12 (cg09698465); cytosine at position 46993515 of human chromosome 10 (cg25995980); cytosine at position 19229767 of human chromosome 9 (cg13469590); and cytosine at position 24229300 of human chromosome 1 (cg24452347). In another more particular example, the means comprised in the kit consist of the means for determining only the methylation status of the thirty-two cytosines in CpG sites.

In another aspect, the invention relates to the use of the kit of the invention for carrying out any of the in vitro methods of the first and second aspects, and their corresponding particular embodiments.

The in vitro methods of the invention provide information regarding the type of response to CAR T-cell therapy and the outcome. In one embodiment, the methods of the invention further comprise the steps of (i) collecting the said information regarding the response and outcome, and (ii) saving the information in a data carrier.

In the sense of the invention a “data carrier” is to be understood as any means that contain meaningful information data for the he prediction of response to CAR T-cell therapy and its outcome, such as paper. The carrier may also be any entity or device capable of carrying the prediction data. For example, the carrier may comprise a storage medium, such as a ROM, for example a CD ROM or a semiconductor ROM, or a magnetic recording medium, for example a floppy disc or hard disk. Further, the carrier may be a transmissible carrier such as an electrical or optical signal, which may be conveyed via electrical or optical cable or by radio or other means. When the prediction/outcome data are embodied in a signal that may be conveyed directly by a cable or other device or means, the carrier may be constituted by such cable or other device or means. Other carriers relate to USB devices and computer archives. Examples of suitable data carrier are paper, CDs, USB, computer archives in PCs, or sound registration with the same information.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### Example 1. Determination of DNA Methylation Markers Indicative of Complete Response to Autologous CAR T-Cell Therapy. Assay with a First Discovery Cohort (n=43)

Following sections disclose a way to carry out the method of the invention for determining if a subject suffering from a B-cell malignancy is a good candidate to autologous CART19 therapy (therapy with CAR T-cells wherein the CAR recognizes antigen CD19 in tumours).

### Methods

### Patients and Samples:

Peripheral-blood mononuclear cells (PBMCs) from a fresh leukapheresis product were isolated from patients with relapsed or refractory (R/R) B-cell malignancy, for which CD19 CAR T-cell therapy (CART19 therapy) was recommended. Patient material was obtained as part of a previously reported clinical trial (NCT02772198 approved by the Sheba Medical Center IRB and the Israeli Ministry of Health (Jacoby et al. Locally produced CD19 CAR T-cells leading to clinical remissions in medullary and extramedullary relapsed acute lymphoblastic leukemia. Am J Hematol; 93: 1485-92; and Itzhaki et al., Head-to-head comparison of in-house produced CD19 CAR-T cell in ALL and NHL patients. J Immunother Cancer 2020; 8: e000148). The clinical characteristics of the studied patients with B-cell malignancy are summarized in Table 1. The isolated peripheral-blood mononuclear cells (PBMCs) were activated in T-cell medium. On day 2 of culture, activated cells were transduced with the CD19 CAR retrovirus. This construct comprised the variable regions of anti-CD19 monoclonal antibody FMC63 (scFV) fused to the CD28 costimulatory domain and to the CD3 zeta chain, which were cloned into a mouse stem-cell virus gamma-retroviral (MSGV) backbone. CAR-T-cells were then further expanded in IL-2 containing T-cell medium until day 9-10. High molecular weight DNA was extracted from paired T-cell samples consisting of CART19 untransduced and transduced T-cells before infusion into patients.

TABLE 1
Clinicopathological characteristics of the B-cell
malignancy patients treated with CART19 cells

| | | |
|---|---|---|
| | Discovery cohort | Subset from the |
| | for differentially | discovery cohort |
| | methylated | used or |
| | CpGs analysis | survival analysis |
| | (N = 43) | (N = 34) |
| Characteristic | N (%) | N (%) |

Sex

| | | | | |
|---|---|---|---|---|
| Male | 23 | (53%) | 19 | (56%) |
| Female | 20 | (47%) | 15 | (44%) |

| | | |
|---|---|---|
| Age (years) | | |
| Median | 39 | 43.5 |
| Range | 7-70 | 7-70 |

| | | | | |
|---|---|---|---|---|
| <18 | 10 | (23%) | 4 | (12%) |
| 18-29 | 6 | (14%) | 6 | (18%) |
| 30-59 | 19 | (44%) | 16 | (47%) |
| >60 | 8 | (19%) | 8 | (24%) |

Diagnostic

| | | | | |
|---|---|---|---|---|
| B-ALL | 13 | (30%) | 6 | (18%) |
| B-NHL | 30 | (70%) | 28 | (82%) |
| DLBCL | 17 | (40%) | 15 | (44%) |
| PMBCL | 9 | (21%) | 9 | (26%) |
| FL | 2 | (5%) | 2 | (6%) |
| Burkitt | 1 | (2%) | 1 | (3%) |
| MCL | 1 | (2%) | 1 | (3%) |

Response

| | | | | |
|---|---|---|---|---|
| CR | 16 | (37%) | 10 | (29%) |
| PR | 13 | (30%) | 11 | (32%) |
| SD | 5 | (12%) | 5 | (15%) |
| PD | 9 | (21%) | 8 | (24%) |

CRS

| | | | | |
|---|---|---|---|---|
| Grade 0 | 7 | (16%) | 6 | (18%) |
| Grade 1 | 25 | (58%) | 23 | (68%) |
| Grade 2 | 4 | (9%) | 1 | (3%) |
| Grade 3 | 5 | (12%) | 2 | (6%) |
| Grade 4 | 2 | (5%) | 2 | (6%) |

ICANS

| | | | | |
|---|---|---|---|---|
| Grade 0 | 22 | (51%) | 19 | (56%) |
| Grade 1 | 8 | (19%) | 8 | (24%) |
| Grade 2 | 3 | (7%) | 1 | (3%) |
| Grade 3 | 6 | (14%) | 4 | (12%) |
| Grade 4 | 4 | (9%) | 2 | (6%) |

### DNA Methylation Procedure and Analysis:

The DNA methylation status of the CART19 untransduced and transduced T-cells from each patient was established using the Infinium MethylationEPIC Array (approximately 850,000 CpG sites) following the manufacturer's instructions for the automated processing of arrays with a liquid handler (Illumina Infinium HD Methylation Assay Experienced User Card, Automated Protocol 15019521 v01), as previously described (Moran et al., Validation of a DNA methylation microarray for 850,000 CpG sites of the human genome enriched in enhancer sequences. Epigenomics 2016; 8: 389-99). DNA methylation in CART19 untransduced and transduced cells was compared. The methylation score of each CpG was represented as a beta value, with a differential CpG beta value >0.33 used as a cut-off. Gene functional annotation by gene-set enrichment analysis (GSEA) was performed using the Gene Ontology (GO) biological process (BP), Reactome and KEGG pathways. Significance of associations between differential DNA methylation status and clinical characteristics was estimated by Fisher's exact test. Samples were clustered in an unsupervised manner using CpG methylation beta values and hierarchical clustering protocols with the complete method for agglomerating Euclidean distances. The EPICART DNA methylation signature (32 cytosines in CpG sites previously disclosed) was obtained using a trained supervised classification model based on a random forest for predicting clinical response. The classification model was optimized by tuning parameters (best model with four variables randomly sampled as candidates at each split, and growing 500 trees) with 10-fold cross-validation, repeated three times. The model performance was assessed using the receiver operating characteristic (ROC) curve of the resamples (Area Under Curve [AUC] mean=0.91, 95% CI=0.85-0.97). To assess EPICART signature enrichment in different T-cell subtypes, BLUEPRINT DNA methylation data for these populations was downloaded (http://blueprint-epigenome.eu) and signal values were compared with EPICART using unsupervised hierarchical clustering analysis and Fisher's exact test. The DNA methylation status of the retroviral CAR vector was determined by pyrosequencing using PyroMark Q96 system. PrePCR was carried out using IMMOLASE (Bioline, USA) hot-start DNA polymerase in a touchdown PCR reaction. For quality control, samples were run on agarose gels to validate the presence of a single sharp band of the expected size with no extra background-amplified products present. Primers were designed using Qiagen's Pyromark Assay Design 2.0 software.

### Clinical Statistical Analysis:

Assay results were compared with patient outcomes in a double-blind manner. The significance of the differences between distributions of the groups was estimated with Fisher's exact test. Event-free survival (EFS) was defined as the time from the start of CART19 treatment until the first occurrence of progression, relapse, or death. Overall survival (OS) was defined as the time from the start of CART19 treatment until death. The Kaplan-Meier method was also used to estimate the EFS and OS, the differences between the groups being calculated with the log-rank test. Hazard ratios (HRs) from univariate Cox regressions were used to determine the association between clinicopathological features and survival.

### Results

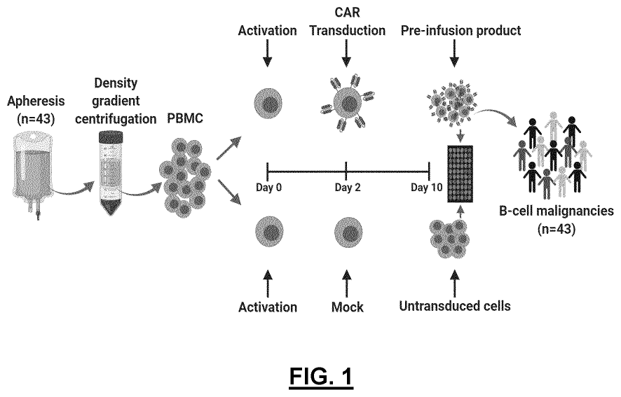

The DNA methylation landscape of untransduced and transduced pre-infusion T-cells for the CD19 CAR retrovirus in 43 patients suffering B-cell malignancies was studied (FIG. 1). This way an epigenomic profile could be obtained. In the 43 patients with B-cell malignancy, DNA methylation levels differed between CART19 untransduced and transduced cells at 1,005 CpG sites. The CpG sites that were distinctly methylated in CAR-positive and CAR-negative T-cells are shown in Table 51 (at the end of the Examples in this description). The distribution of these CpG sites in the human genome was as follows. They were associated with known genes in 74.7% (751 of 1,005) of cases and, of these, the differential epigenetic sites were located within a defined regulatory region in 45.9% (345 of 751) of cases. To better characterize the discovered group of 751 genes that underwent DNA methylation changes upon CAR transduction, gene functional annotation by GSEA using Gene Ontology (GO signature) collections was performed. The most overrepresented GO biological processes and KEGG and Reactome pathways proved to be the “T-cell receptor signaling pathway”, “Pathways in cancer” and “Mitotic metaphase and anaphase”, respectively. These processes are highly important in potential CART19 anti-B malignant cell activity, since they are related to T-cell biology, cellular transformation networks and proliferation capacity. Fisher's exact test was used to identify the associations between the DNA methylation status of the 1,005 CpG sites identified in the CART19-transduced cells before infusion into the patient and each of three outcome events: clinical response; the appearance of adverse effects of cytokine release syndrome (CRS); and/or immune effector cell-associated neurotoxicity syndrome (ICANS). For the contingency tables, clinical response was categorized as complete response (CR) vs. non-complete response (partial response [PR]+stable disease [SD]+progression of the disease [PD]); CRS was divided into Grade 0 vs. Grades 1-4; and (CANS was split into Grade 0 vs. Grades 1-4. Among the epigenomic loci studied, found 54 CpG sites (5.3% of the 1,005 sites identified) were found at which the DNA methylation levels were significantly associated with the clinical variables assessed. The DNA methylation status of 32, 12 and 18 CpG sites was associated, respectively, with complete clinical response (Table 3, see above), the occurrence of CRS (Table 4), and the presence of (CANS (Table 5). To reinforce the potential translational value of the epigenetic events identified in patient T-cells engineered to express the CART19, the DNA methylation status for each set of CpGs associated with each clinical outcome was also plotted in an unsupervised manner (Data not shown). For each variable, the hierarchical clustering distinguished two branches that were significantly enriched with respect to each condition: complete response (Fisher's exact test, P=0.0003), CRS (Fisher's exact test, P=0.001) and (CANS (Fisher's exact test, P=0.005), providing further evidence of the significance of these markers in the described clinical events.

TABLE 4
Annotation of the 12 CpGs correlated with CRS

| | Chromosomal | Associated | CRS |
|---|---|---|---|
| Probe ID | position (hg19) | gene | P-value |
| cg09554300 | chr1: 201123894 | TMEM9 | 0.008 |
| cg14416782 | chr3: 45505849 | LARS2 | 0.010 |
| cg21847720 | chr8: 2075777 | MYOM2 | 0.015 |
| cg14538944 | chr2: 218340518 | DIRC3 | 0.023 |
| cg19627006 | chr2: 85637673 | CAPG | 0.023 |
| cg22836400 | chr6: 10415636 | TFAP2A | 0.023 |
| cg20017856 | chr14: 29990921 | MIR548AI | 0.024 |
| cg07199183 | chr15: 100879199 | ADAMTS17 | 0.030 |
| cg11005552 | chr10: 105648138 | OBFC1 | 0.038 |
| cg14755254 | chr8: 637813 | ERICH1 | 0.038 |
| cg19196401 | chr6: 110721138 | DDO | 0.040 |
| cg15612205 | chr12: 130516192 | Not described | 0.045 |

Annotation retrieved from the Infinium MethylationEPIC Array manifest. Probe ID: unique identifier from the Illumina CG database. Chromosomal position (hg19): chromosomal coordinates of the CpG (build hg19). Associated gene: target gene name from the UCSC database. The P-value of the CRS is derived from the Fisher's exact test (CRS grade 0 vs grades 1/2/3/4).

TABLE 5
Annotation of the 18 CpGs correlated with ICANS

| | Chromosomal | Associated | ICANS |
|---|---|---|---|
| Probe ID | position (hg19) | gene | P-value |
| cg27272679 | chr8: 65294635 | Not described | 0.0005 |
| cg07199183 | chr15: 100879199 | ADAMTS17 | 0.002 |
| cg09992216 | chr11: 32353565 | Not described | 0.005 |
| cg14215970 | chr9: 124132919 | STOM | 0.007 |
| cg24267358 | chr19: 42299379 | CEACAM3 | 0.01 |
| cg25606201 | chr5: 180614858 | Not described | 0.01 |
| cg25268100 | chr10: 134457731 | INPP5A | 0.012 |
| cg12610471 | chr10: 22634199 | SPAG6 | 0.015 |
| cg12197459 | chr17: 686450 | GLOD4; RNMTL1 | 0.021 |
| cg12504912 | chr14: 90081872 | FOXN3 | 0.021 |
| cg21390512 | chr8: 127568850 | FAM84B | 0.021 |
| cg02978297 | chr1: 94057587 | BCAR3 | 0.034 |
| cg14683065 | chr10: 134149184 | LRRC27 | 0.034 |
| cg01412970 | chr17: 17109239 | PLD6 | 0.045 |
| cg01664727 | chr21: 36258423 | RUNX1 | 0.046 |
| cg14161159 | chr2: 132481826 | C2orf27A | 0.049 |
| cg15227982 | chr10: 104535854 | C10orf26 | 0.049 |
| cg24365464 | chr1: 190448126 | FAM5C | 0.049 |

Annotation retrieved from the Infinium MethylationEPIC Array manifest. Probe ID: unique identifier from the Illumina CG database. Chromosomal position (hg19): chromosomal coordinates of the CpG (build hg19). Gene name: target gene name from the UCSC database. The P-value of the ICANS is derived from the Fisher's exact test (ICANS grade 0 vs grades 1/2/3/4).

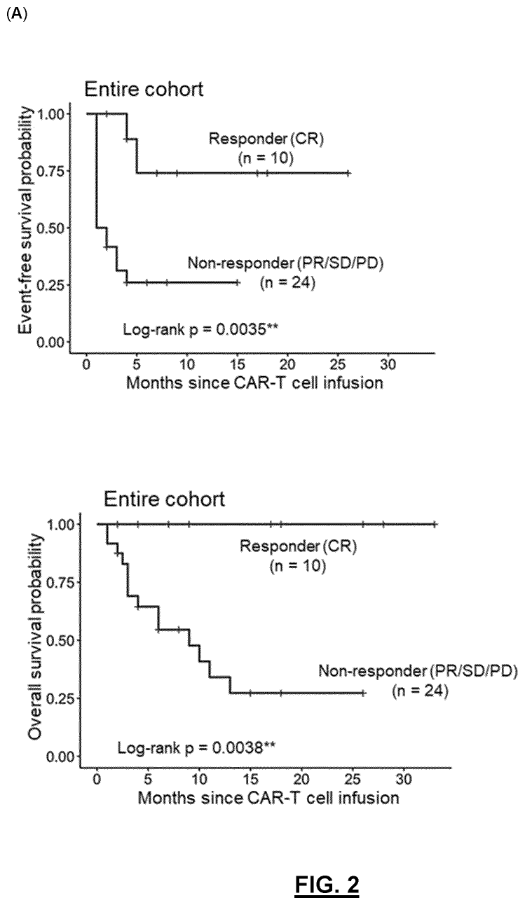

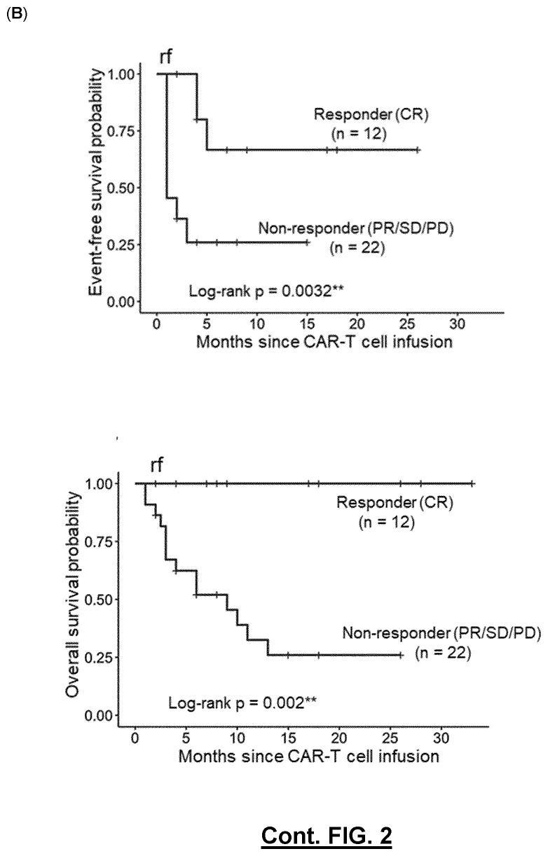

Once it was established that a set of 32 epigenomic loci could discriminate complete clinical response to CART19 treatment in the patients with B-cell malignancy, it was examined whether the DNA methylation markers obtained could also predict EFS and OS in these cases. Among the initial set of 43 cases, these clinical parameters were studied in 34 patients, having excluded 9 cases due to loss to follow-up (N=3) or for having undergone hematopoietic stem-cell transplantation (HSCT) after CART19 therapy (N=6). The clinicopathological characteristics of the patients evaluated for EFS and OS are also listed in Table 1. In this regard, the presence of complete clinical response in this cohort was associated with enhanced EFS (HR=0.15, P=0.002; 95% CI=0.034-0.651; log-rank P=0.0035) and improved OS (HR=0.06, P=0.002; 95% CI=0.001-0.44; log-rank P=0.0038) (FIG. 2A). It is of particular note that when the 32 methylation sites that correlated with complete response were selected (Table 3) to train a supervised classification model based on random forest, an epigenetic signature (referred to hereafter as the EPICART signature) was obtained that was significantly associated with EFS (HR=0.18, P=0.002; 95% CI=0.052-0.634; log-rank P=0.0032) (FIG. 2B) and OS (HR=0.05, P=0.0008; 95% CI=0.0004-0.386; log-rank P=0.002) (FIG. 2B). Remarkably, the EPICART-positive signature kept its clinical value when the patients with B-cell malignancy who had been treated with CART19 were subdivided into B-acute lymphoblastic leukemias or B-lymphomas; both groups exhibited improved EFS and OS (data not shown). The EPICART signature was not associated with the B-cell malignancy type (ALL vs NHL) (Fisher's exact test, P=0.641) or the age of the patient (pediatric vs adult) (Fisher's exact test, P=0.6015).

Taking advantage of the carefully dissected DNA methylation patterns of the different T-cell populations available from the International Human Epigenome Consortium (IHEC), a molecular dissection of the T-cell classes in our EPICART signature was undertaken. The EPICART-positive signature, which was associated with improved clinical outcome, identified CART19 cells enriched in CD4 and CD8 naive-like or early memory phenotype T-cells (P=0.0001). Conversely, EPICART-negative CART19 cells were enriched in more committed and differentiated lineages, such as effector memory CD4 and CD8 T-cells, and terminally differentiated effector memory CD8 T-cells (P=0.01). These results are consistent with the adoptive cell therapy concept that naive-like or early memory T-cells (such as the T-stem cell memory subclass) can outperform effector T-cells.

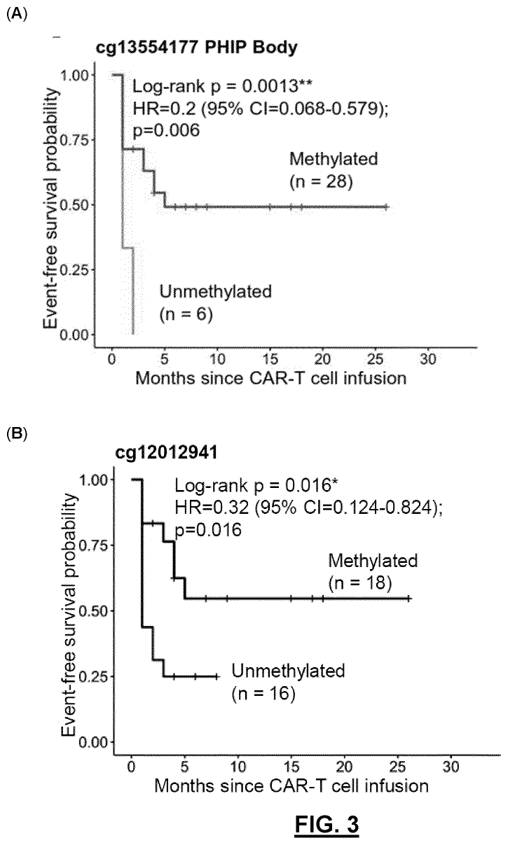

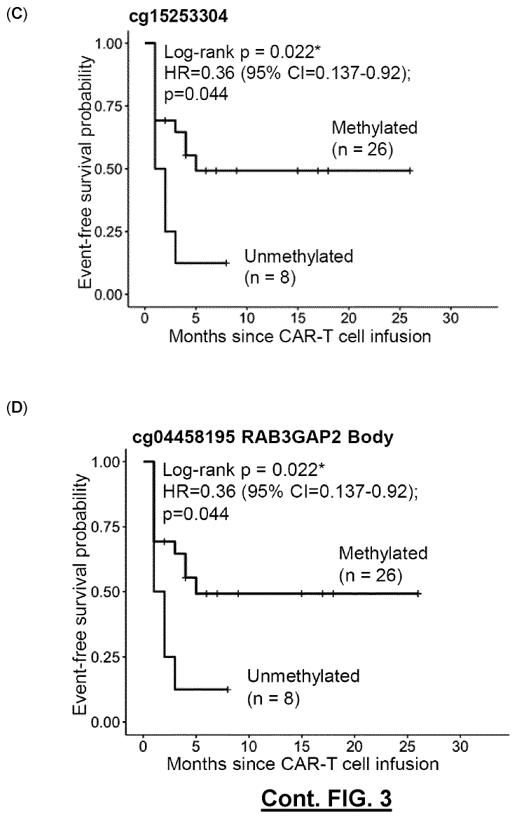

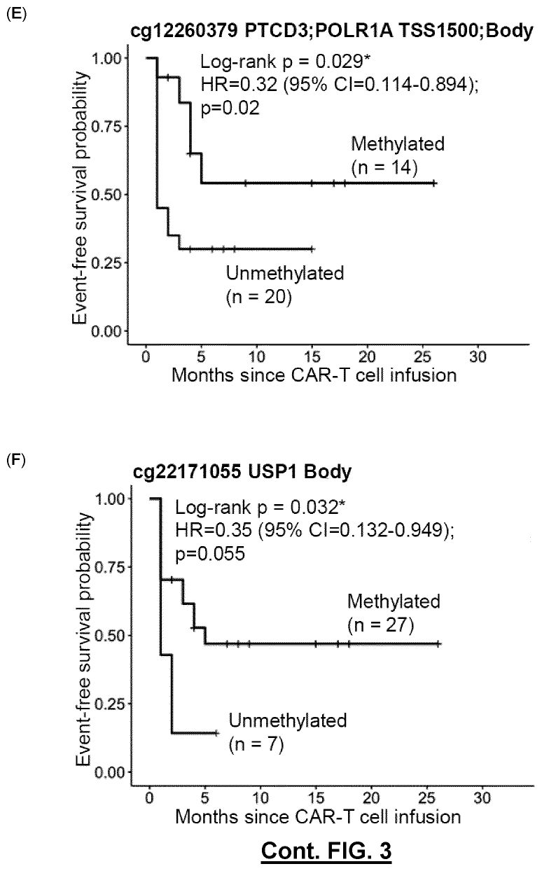

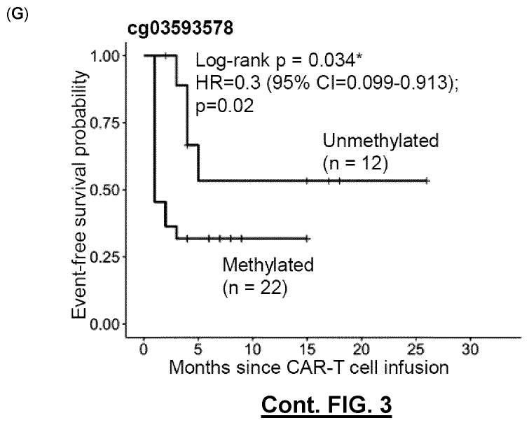

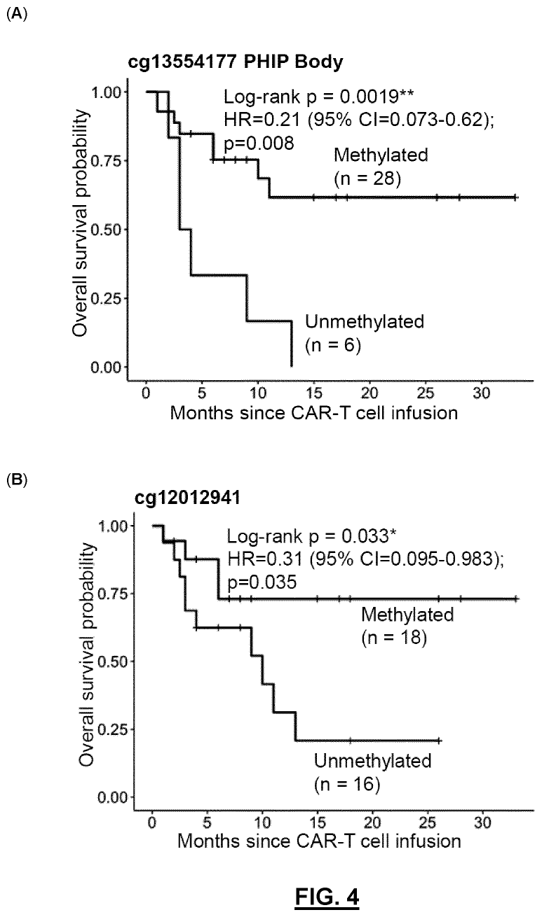

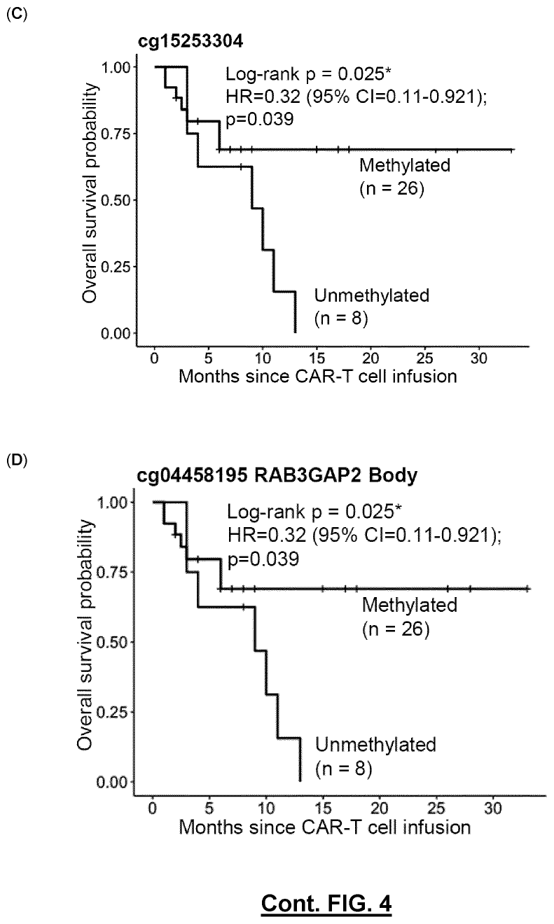

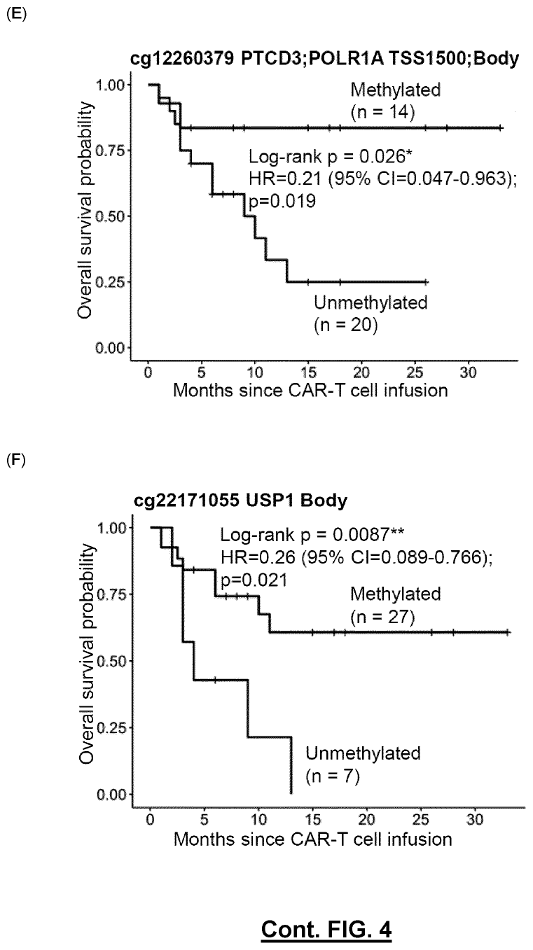

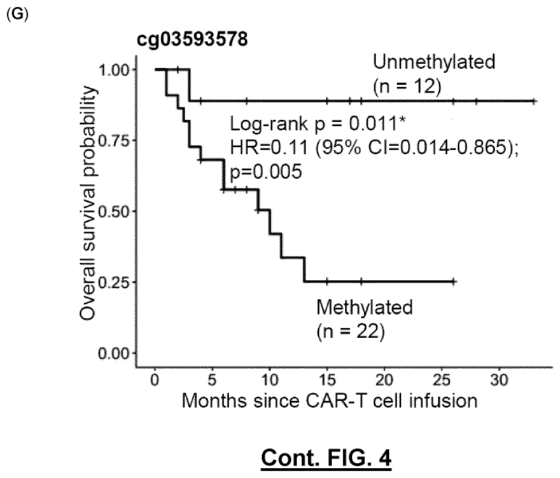

Obtaining the EPICART DNA methylation (32 CpG sites) signature for CART19 preinfused T-cells that predicts extended EFS and OS is extremely useful. Inventors also identified and developed a smaller set of DNA methylation biomarkers that in addition simplified the analysis. Those DNA methylation sites of the 32 defining the EPICART signature that were individually associated with both improved EFS and longer OS were tested. Seven epigenomic loci that were detected that, analyzed alone, were also associated with better EFS and OS. The features of these CpG sites are summarized in Table 2 (CpG sites in bold in Table 3) and the corresponding Kaplan-Meier curves for EFS and OS are shown in FIG. 3 (A-G) and FIG. 4 (A-G), respectively.

TABLE 2
Annotation of the 7 CpGs correlated with Complete Response
and with significant improvement in EFS and OS.

| | | | Complete | | |
|---|---|---|---|---|---|
| | Chromosomal | Associated | Response | EFS | OS |
| Probe ID | position (hg19) | gene | P-value | P-value | P-value |
| cg12260379 | chr2: 86332162 | PTCD3; POLR1A | 0.0001 | 0.029 | 0.02 |
| cg12012941 | chr1: 188676237 | Not described | 0.0008 | 0.016 | 0.033 |
| cg03593578 | chr2: 45028225 | Not described | 0.01 | 0.034 | 0.011 |
| cg04458195 | chr1: 220414164 | RAB3GAP2 | 0.016 | 0.022 | 0.025 |
| cg15253304 | chr6: 209809 | Not described | 0.016 | 0.022 | 0.025 |
| cg22171055 | chr1: 62905816 | USP1 | 0.018 | 0.032 | 0.009 |
| cg13554177 | chr6: 79780164 | PHIP | 0.035 | 0.001 | 0.002 |

Annotation retrieved from the Infinium MethylationEPIC Array manifest. Probe ID: unique identifier from the Illumina CG database. Chromosomal position (hg19): chromosomal coordinates of the CpG (build hg19). Associated gene: target gene name from the UCSC database. The P-value of the Complete Response is derived from the Fisher's exact test (CR vs NR/SD/PD). The P-value of event-free survival (EFS) and overall survival (OS) is derived from the log-rank test in Kaplan-Meier curves.

It is of note that the five genes associated with these seven DNA methylation loci are involved in regulating protein levels. Thus, for example, gene of Ubiquitin carboxyl-terminal hydrolase 1 (USP1; UniprotKB 094782), gene of Rab3 GTPase-activating protein non-catalytic subunit (RAB3GAP2; UniprotKB Q9H2M9) and gene of PH-interacting protein (PHIP; UniprotKB Q8WWQ0) were involved in protein degradation by the ubiquitin pathway, and gene of Pentatricopeptide repeat domain-containing protein 3, mitochondrial (PTCD3; UniprotKB Q96EY7) and gene of DNA-directed RNA polymerase I subunit RPA1 (POLR1A; UniprotKB 095602) played a role in protein production at the ribosomes. The case of USP1 could be particularly relevant because it controls the protein expression levels of Inhibitor of DNA Binding 2 (ID2), a gene that is overexpressed in the CD8 T-cells of infused CART19 patients who do not achieve a complete clinical response (Deng et al., Characteristics of anti-CD19 CAR T cell infusion products associated with efficacy and toxicity in patients with large B cell lymphomas. Nat Med 2020; published online October 5. https://doi.org/10.1038/s41591-020-1061-7).

Two additional DNA methylation analyses were performed. T-cells were transduced with a CD19 CAR retrovirus based on a mouse stem-cell virus gamma-retroviral (MSGV) model. This type of retroviral vector is itself vulnerable to epigenetic silencing via DNA methylation. Inventors examined whether a distinct DNA methylation status of the retrovirus in the transduced T-cell could also influence clinical outcome. Pyrosequencing analyses of multiple sites of the retroviral vector, including the 5′ long terminal repeat (LTR) promoter region, showed, in the entire cohort (n=43), an unmethylated status of MSGV in all CART19 pre-infused cells (Data not shown). For this reason, it was concluded that the measurement could not account for any of the differential clinical outcomes.

Inventors also propose herewith an additional CpG site, a part of the 7 correlating with good (i.e. high) EFS and OS and CR, and the 32 correlating with CR and significantly associated with EFS and OS. This additional locus corresponds to cytosine at position 28725934 (cg08544307) of human chromosome 8. Methylation at this cytosine was associated with both enhanced EFS and longer OS. The DNA methylation locus was within the INTS9 gene, a member of the integrator family that regulates hematopoiesis and leukemogenesis. Thus, it is a unique example of a biomarker that, without a direct association with the complete clinical response to CART19 treatment, would partially determine the long-term effects of adoptive cell therapy.

Therefore, inventors have provided a helpful tool for hematologists and oncologists to be able to rely on predictive biomarkers for CART19 complete response and clinical outcome, in addition to indicators of the likelihood of adverse reactions to the treatment. This invention supposes, moreover, a log-felt need since CART19 treatment in particular, and the whole field of adoptive cell therapy in general, are almost entirely lacking in molecular factors that can be categorized as biomarkers of this type. All the herewith provided results show that the use of DNA methylation profiling in CART19-transduced T-lymphocytes also provides a consistent readout associated with clinical response events, undesirable side-effects, EFS, and OS in patients with relapsed/refractory (R/R) B-cell malignancies who have received this class of cell therapy. These results further strengthen the notion that research into specific DNA and RNA profiles and components of the cells used in adoptive cell therapy, in addition to the knowledge it yields about the tumor and the molecular background of the host patient, is of great value for determining treatment success and its potential adverse effects. It is also worth noting that the FDA-approved CART19 treatment with axicabtagene ciloleucel (Yescarta) also uses a retrovirus and that CART19 exemplified in this invention is also a retroviral vector. No traces of DNA methylation in the construct of the transduced pre-infusion cells were detected.

As above indicated, all these proposed DNA methylation sites as predictors of CART19 clinical efficacy in B-cell malignancies, give relevant information for the further external intervention in the ex vivo growth of the T-cells, and their transduction with the CAR, to optimize the production of re-engineered cells with greater therapeutic capacity. Examples of these interventions include using the DNA hypomethylating agents approved for clinical use in hematological malignancies, such as decitabine or vidaza. Decitabine has been reported to enhance the anti-leukemia efficacy of CD123-targeted CAR T-cells in preclinical models (You et al., Decitabine-mediated epigenetic reprograming enhances anti-leukemia efficacy of CD123-targeted chimeric antigen receptor T-cells. Front Immunol 2020; 11: 1787). These drugs can, in particular examples, be complemented with compounds targeting other elements of the epigenetic setting, because DNA methylation events are commonly associated with shifts in histone modifications; and changes in chromatin-accessible sites upon CAR-T transduction has also been reported. Thus, histone deacetylase inhibitors such as vorinostat, and histone methyltransferase inhibitors such as the EZH2 inhibitor tazverik, approved for use for treating subtypes of lymphomas and sarcomas, are also proposed, in the context of the manufacture of CART19, for enhancing the activity of the cells produced.

### Example 2. Determination of DNA Methylation Markers Indicative of Complete Response to Autologous CAR T-Cell Therapy. Assay with a Large Discovery Cohort (n=79) and with a Validation Cohort (n=35)

With the aim of obtaining robust results, the analysis as indicated in example 1 was repeated with a large validation cohort (n=79). In addition, a validation cohort was also tested (n=35).

### Study Design

Patients were eligible to enter the study if they had a relapsed or refractory (R/R) B-cell malignancy for which CART19 therapy was recommended. Patient CD19-engineered T-cells from 114 cases were obtained from three academic clinical trials: NCT03144583 (Ortíz-Maldonado V, Rives S, Castellà M, et al. CART19-BE-01: A Multicenter Trial of ARI-0001 Cell Therapy in Patients with CD19+Relapsed/Refractory Malignancies. Mol Ther. 2021; 29(2):636-644), NCT02772198 (references in Example 1) and NCT03373071 (Quintarelli C, Guercio M, Manni S, et al. Strategy to prevent epitope masking in CAR.CD19+B-cell leukemia blasts. J. Immunother. Cancer. 2021; 9(6):e001514.). Written informed consent was obtained, and the Sheba Medical Center IRB and the Israeli Ministry of Health, the Research Ethics Comitee (Celm) of the Hospital Clinic, and the IRB of Bambino Gesii Children Hospital, respectively, provided study approval. The clinical characteristics of the studied 114 patients are summarized in Table C below. High molecular weight DNA was extracted from all cases before CART19 infusion into patients.

TABLE C
Clinicopathological characteristics of the B-cell
malignancy patients treated with CART19 cells

| | Entire | | Discovery | | Validation | |
|---|---|---|---|---|---|---|
| | cohort | | cohort | | cohort | |
| | (n = 114) | | (n = 79) | | (n = 35) | |
| Characteristic | n (%) | | n (%) | | n (%) | |
| Sex | | | | | | |
| Male | 68 | (59.6) | 41 | (51.9) | 27 | (77.1) |
| Female | 46 | (40.4) | 38 | (48.1) | 8 | (22.9) |
| Age (years) | | | | | | |
| Median | 24 | | 22 | | 27 | |
| Range | 3-70 | | 3-70 | | 4-70 | |
| <18 | 42 | (36.8) | 32 | (40.5) | 10 | (28.6) |
| 18-29 | 27 | (23.7) | 16 | (20.3) | 11 | (31.4) |
| 30-59 | 34 | (29.8) | 26 | (32.9) | 8 | (22.9) |
| >60 | 11 | (9.6) | 5 | (6.3) | 6 | (17.1) |
| Diagnostic | | | | | | |
| B-ALL | 77 | (67.5) | 53 | (67.1) | 24 | (68.6) |
| B-NHL | 37 | (32.5) | 26 | (32.9) | 11 | (31.4) |
| DLBCL | 20 | (17.5) | 13 | (16.5) | 7 | (20.0) |
| PMBCL | 11 | (9.6) | 9 | (11.4) | 2 | (5.7) |
| FL | 4 | (3.5) | 3 | (3.8) | 1 | (2.9) |
| Burkitt | 1 | (0.9) | 0 | (0.0) | 1 | (2.9) |
| MCL | 1 | (0.9) | 1 | (1.3) | 0 | (0.0) |
| Response | | | | | | |
| CR | 74 | (64.9) | 50 | (63.3) | 24 | (68.6) |
| PR | 16 | (14) | 11 | (13.9) | 5 | (14.3) |
| SD | 9 | (7.9) | 6 | (7.6) | 3 | (8.6) |
| PD | 15 | (13.2) | 12 | (15.2) | 3 | (8.6) |
| CRS | | | | | | |
| Grade 0 | 41 | (36.0) | 28 | (35.4) | 13 | (37.1) |
| Grade 1 | 46 | (40.4) | 33 | (41.8) | 13 | (37.1) |
| Grade 2 | 13 | (11.4) | 10 | (12.7) | 3 | (8.6) |
| Grade 3 | 8 | (7.0) | 4 | (5.1) | 4 | (11.4) |
| Grade 4 | 4 | (3.5) | 2 | (2.5) | 2 | (5.7) |
| Grade 5 | 2 | (1.8) | 2 | (2.5) | 0 | (0.0) |
| ICANS | | | | | | |
| Grade 0 | 87 | (76.3) | 59 | (74.7) | 28 | (80.0) |
| Grade 1 | 11 | (9.6) | 8 | (10.1) | 3 | (8.6) |
| Grade 2 | 5 | (4.4) | 4 | (5.1) | 1 | (2.9) |
| Grade 3 | 6 | (5.3) | 4 | (5.1) | 2 | (5.7) |
| Grade 4 | 5 | (4.4) | 4 | (5.1) | 1 | (2.9) |
| Grade 5 | 0 | (0.0) | 0 | (0.0) | 0 | (0.0) |

| Origin of the CAR-Ts | | | | |
|---|---|---|---|---|
| NCT02772198 | 43 (37.7) | 30 (38.0) | 13 | (37.1) |
| NCT03144583 | 45 (39.5) | 31 (39.2) | 14 | (40.0) |
| NCT03373071 | 26 (22.8) | 18 (22.8) | 8 | (22.9) |

ALL: acute lymphoblastic leukemia; NHL: non-Hodgkin lymphoma; DLBCL: diffuse large B-cell lymphoma; PMBCL: primary mediastinal B-cell lymphoma; FL: follicular lymphoma; MCL: mantle cell lymphoma; CR: complete response; PR: partial response; SD: stable disease; PD: progression of the disease; CRS: cytokine release syndrome; ICANS: immune effector cell-associated neurotoxicity syndrome.

### DNA Methylation Procedure and Analysis:

The DNA methylation status of the CART19 cells from each patient was established using the Infinium MethylationEPIC Array (Morat et al., supra). DNA methylation data are available at GEO repository (GSE179414, reviewer token: wbkdquweltcvdgj https://www.ncbi.nlm.nih.govigeo/query/acc.cgi?acc=GSE179414). EPICART18 DNA methylation signature was obtained using a trained supervised classification model based on ridge regularized logistic regression to predict clinical response. The classification model was optimized by tuning parameters (best performance with alpha=0 from ridge regression and regularization parameter lambda=0.03) with 10-fold cross-validation, repeated three times. The model performance was assessed using the receiver operating characteristic (ROC) curve of the resamples (Area Under Curve [AUC] mean=0.83, 95% CI=0.75-0.91). Flow cytometry analysis was used for validation. DNA methylation status of specific CpG sites was validated by pyrosequencing and bisulfite genomic sequencing of multiple clones. Real time quantitative (qRT-PCR) and western-blot was used to assess gene expression.

### Clinical Statistical Analysis

Assay results were compared with patient outcomes in a double-blind manner. The significance of the differences between distributions of the groups was estimated with Fisher's exact test. Event-free survival (EFS) was defined as the time from the start of CART19 treatment until the first occurrence of progression, relapse, or death. Overall survival (OS) was defined as the time from the start of CART19 treatment until death. The Kaplan-Meier method was also used to estimate the EFS and OS, the differences between the groups being calculated with the log-rank test. Hazard ratios (HRs) from univariate Cox regressions were used to determine the association between clinicopathological features and survival.

### Results

### The Epigenomic Landscape of CART19 Cells:

To discover an epigenomic profile associated with B-cell malignancy cases who would gain clinical benefit from CART19 treatment, the DNA methylation landscape of untransduced and transduced pre-infusion T-cells for the CD19 CAR retrovirus in 43 patients from the NCT02772198 clinical trial was studied (Example 1). This set of cases included 30 NHL (28 adult and 2 pediatric patients) and 13 ALL (8 pediatric and 5 adult patients). In this initial set, the methylation status of around 850,000 CpG sites was interrogated. In the 43 patients with B-cell malignancy, DNA methylation levels differed between CART19 untransduced and transduced cells at 984 CpG sites (all included in Table S1 below). Among these differential CpG sites, 53% (519 of 984) were hypermethylation events at the CART19 transduced cells vs the untransduced, whereas 47% (465 of 984) were hypomethylation changes. The CpG methylation content of these 984 sites was not distinct between CD4 and CD8 T-cells (Wilcoxon-Mann-Whitney test analysis, p=0.73). The genomic distribution of these CpG sites was the following: They were associated with known genes in 75.1% (739 of 984) of cases and, of these, were located within a defined regulatory region in 45.9% (339 of 739) of cases. Gene set enrichment analysis using gene ontology collections showed that the most overrepresented biological processes and KEGG and Reactome pathways were the “T-cell receptor signaling pathway”, “Pathways in cancer” and “Separation of sister chromatids”, respectively. Using only CpG sites for regulatory regions, the most overrepresented categories were “T-cell receptor signaling pathway” and “Transcriptional regulation by Runx3”; whereas using only gene body-sites, the most overrepresented categories were “Homophilic cell adhesion via plasma membrane adhesion molecules” and “Separation of sister chromatids”.

T-cells transduced with CD19 CAR retroviruses could themselves be vulnerable to DNA methylation silencing (20). Thus, it was examined whether a distinct DNA methylation status of the retrovirus in the transduced T-cell could also influence clinical outcome. Pyrosequencing analyses of the retroviral vector showed an unmethylated status of the retroviral vector in the CART19 cells.

### Impact of CART19 Epigenetics in Clinical Outcome: The EPICART18 Signature:

Fisher's exact test with correction for multiple hypothesis testing using the false discovery rate (FDR) was applied to identify any association between the DNA methylation status of the 984 CpG sites identified in CART19-transduced cells and the clinical outcomes in 114 B-cell malignancy patients treated with this type of cell therapy (Table C). For the contingency tables, clinical response was categorized as complete response (CR) vs. non-complete response (partial response [PR]+stable disease [SD]+progression of the disease [PD]). For the adverse effects, the guidelines of the American Society for Transplantation and Cellular Therapy wer followed (Lee D W, Santomasso B D, Locke F L, et al. ASTCT Consensus Grading for Cytokine Release Syndrome and Neurologic Toxicity Associated with Immune Effector Cells. Biol Blood Marrow Transplant. 2019; 25(4):625-638): cytokine release syndrome (CRS) was divided into Grade 0 vs. Grades 1-5; and immune effector cell-associated neurotoxicity syndrome (ICANS) was split into Grade 0 vs. Grades 1-5. These cases were divided into a discovery cohort of 79 patients and a validation cohort of 35 patients (Table C). The two cohorts did not show significant differences related to age (pediatric vs adult, Fisher's exact test, P=0.29), origin of the sample (NCT03144583, NCT02772198 and NCT03373071, Fisher's exact test, P=1), type of B-cell malignancy (ALL vs NHL, Fisher's exact test, P=1), clinical response (CR vs PR/SD/PD, Fisher's exact test, P=0.67) and the appearance of CRS (0 vs 1-5, Fisher's exact test, P=1) or ICANS (0 v 1-5, Fisher's exact test, P=0.64). DNA from the CART19-transduced cells infused in each patient was hybridized to the described DNA methylation microarray.

In the discovery cohort (n=79), ther were found 54 CpG sites (5.5% of the 984 sites) at the initial screening by Fisher's exact test for which the DNA methylation levels were significantly associated with clinical variables. The DNA methylation status of 45, 8 and 5 CpG sites was associated, respectively, with CR, CRS (See Table D below), and ICANS (see Table E below). It was then applied to all the identified CpG sites with potential clinical value derived from the Fisher's exact test, the FDR statistical approach that it is used in multiple hypothesis testing to correct for multiple comparisons. It was found that, although the epigenetic loci linked to CRS and ICANS failed this test, 40% (18 of 45) of the CpG sites associated with CR passed the FDR for multiple testing:

TABLE D
Annotation of the 8 CpGs correlated
with Cytokine Release Syndrome (CRS)

| | Chromosomal | Associated | CRS |
|---|---|---|---|
| Probe ID | position (hg19) | gene | p-value |
| cg21847720 | chr8: 2075777 | MYOM2 | 0.014 |
| cg01311063 | chr2: 131058184 | Not described | 0.018 |
| cg00994804 | chr21: 36259383 | RUNX1 | 0.022 |
| cg25606201 | chr5: 180614858 | Not described | 0.033 |
| cg26669806 | chr19: 18899483 | COMP | 0.037 |
| cg24365464 | chr1: 190448126 | FAM5C | 0.039 |
| cg14538944 | chr2: 218340518 | DIRC3 | 0.041 |
| cg22836400 | chr6: 10415636 | TFAP2A | 0.041 |

Annotation retrieved from the Infinium MethylationEPIC Array manifest. Probe ID: unique identifier from the Illumina CG database. Chromosomal position (hg19): chromosomal coordinates of the CpG (build hg19). Associated gene: target gene name from the UCSC database. The P-value of the CRS is derived from the Fisher's exact test (CRS grade 0 vs grades 1-5).

TABLE E
Annotation of the 5 CpGs correlated with Immune Effector
Cell-Associated Neurotoxicity Syndrome (ICANS)

| | Chromosomal | Associated | ICANS |
|---|---|---|---|
| Probe ID | position (hg19) | gene | p-value |
| cg01311063 | chr2: 131058184 | Not described | 0.0004 |
| cg26195366 | chr10: 102242535 | WNT8B | 0.014 |
| cg22534145 | chr20: 23015936 | SSTR4 | 0.037 |
| cg27272679 | chr8: 65294635 | Not described | 0.042 |
| cg27196695 | chr10: 134571377 | INPP5A | 0.046 |

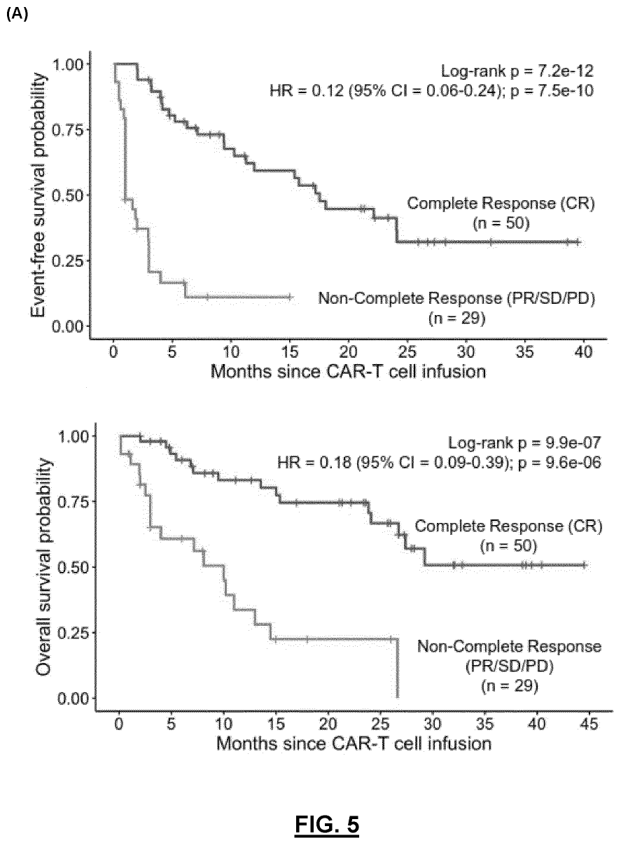

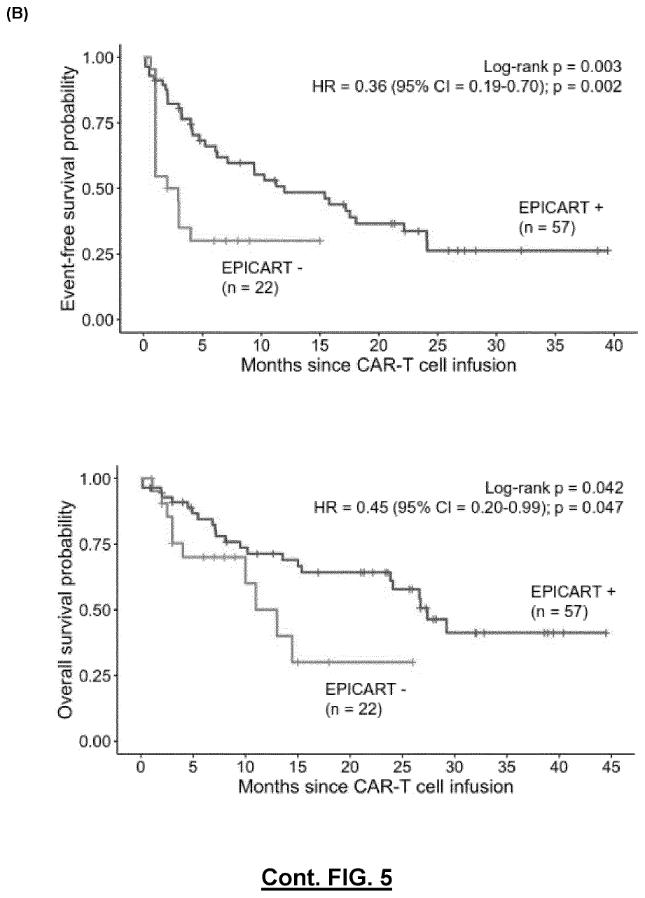

Once established a set of 18 epigenomic loci adjusted by multiple testing could discriminate a CR result following CART19 treatment (See Table B, above, in this description), it was examined whether these sites could also predict EFS and OS in the discovery cohort (n=79). In this regard, the presence of a CR was associated with enhanced EFS and improved OS (FIG. 5). When the 18 methylation sites that correlated with CR were selected, to train a supervised classification model based on ridge regularized logistic regression, it was obtained an epigenetic signature, referred to hereafter as the EPICART18 signature. The use of the EPICART18 signature in the supervised hierarchical clustering for the discovery cohort of CAR-T cases classified patients as those exhibiting CR or non-CR (Fisher's exact test, P=3.3e-13). Most important, the EPICART18 signature was associated with EFS (FIG. 5 B)) and OS (FIG. 5(B)).

Taking advantage of the dissected DNA methylation patterns of the different T-cell populations from the International Human Epigenome Consortium (IHEC) (22), a molecular dissection of the T-cell classes in our EPICART18 signature was undertaken. It was found that the EPICART18-positive signature identified CART19 cells enriched in CD4 and CD8 naive-like or early memory phenotype T-cells (Fisher's exact test, P=0.034). Conversely, EPICART18-negative CART19 cells were enriched in more committed and differentiated lineages, such as effector memory CD4 and CD8 T-cells, and terminally differentiated effector memory CD8 T-cells (Fisher's exact test, P=0.0001). The described population phenotypes assigned by computational projection were validated by flow cytometry analyses in forty-three cases of the discovery cohort where these data were available. The use of the markers CD3, CD45RA and CCR7 to define the population status of naïve T-cells (TN: CD3+CD45RA+CCR7+), central memory T-cells (TCM: CD3+CD45RA-CCR7+), effector memory T-cells (TEM: CD3+CD45RA-CCR7−) and effector T-cells (TEMRA: CD3+CD45RA+CCR7−) confirmed that EPICART-positive CART19 cells were enriched in naïve T-cells/central memory T-cells (Student's t-test, P=0.04), whereas in EPICART-negative cells effector memory T-cells/effector T-cells populations were overrepresented (Student's t-test, P=0.027). Importantly, it was observed that those B-cell malignancy patients receiving CAR-Ts enriched with naïve and central memory T-cells (TN+TCM) showed improved EFS and OS in comparison to those given adoptive cell therapy enriched in effector memory and effector T-cells (TEM+TEMRA) (data not shown). These results are consistent with the adoptive cell therapy concept that naive-like or early memory T-cells can outperform effector T-cells due to the limited niche homing, survival and self-renewal capacity of the effector cells relative to the less committed and more immature T-cells.

Related to any obvious impact on gene expression for the 18 CpG sites that defined the EPICART18 signature, RNA and/or protein for the CART19 cells was not available, thus 105 blood cell lines analyzed for DNA methylation and expression were datamined. It was observed that hypermethylation of those CpGs located in the gene bodies was associated with transcript upregulation (Mann-Whitney-Wilcoxon test, P=1.2e-14 5.8e-15). The presence of gene body hypermethylation accompanied by gene upregulation has been reported. Importantly, using T-cell derived lines from these analyses, it was validated that INPP5A and ECHDC1 gene-body hypermethylation was associated with elevated expression determined; whereas gene-body hypomethylation was associated with gene downregulation. Concordantly, the use of the DNA methylation inhibitor 5-Aza-2′-deoxycytidine in the hypermethylated cell lines downregulated INPP5A and ECHDC1 expression. Furthermore, it was experimentally validated by pyrosequencing and bisulfite genomic sequencing of multiple clones the DNA methylation status of these CpG sites in EPICART18-positive and negative patients. Further data-mining of the T-cell derived lines showed that hypermethylation of 5′-end CpG sites was mostly associated with transcript downregulation. An illustrative example is the 5′-UTR CpG hypermethylation of FOXN3, a candidate tumor suppressor for T-cell acute lymphoblastic leukemia.

EPICART18 Validation and Single Loci Associated with Clinical Course:

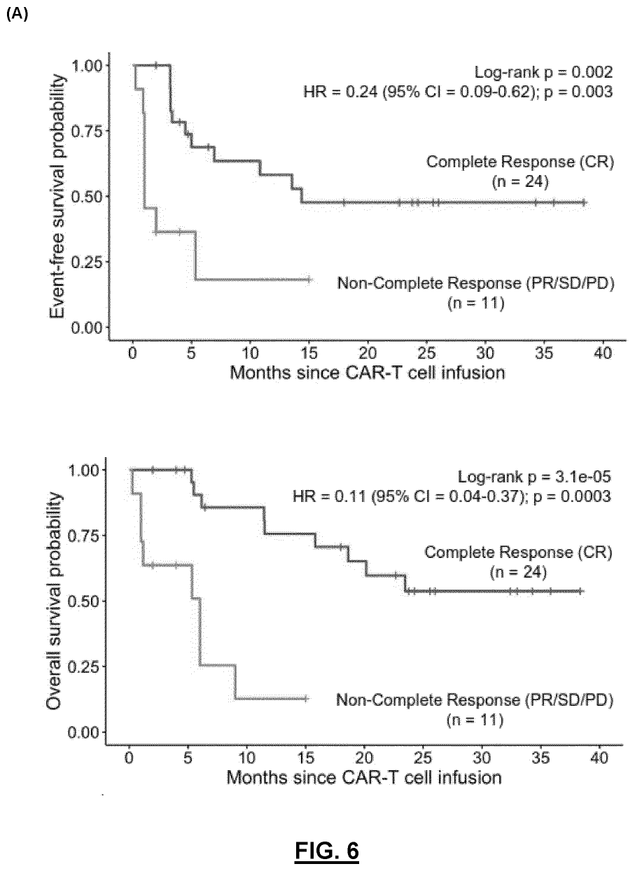

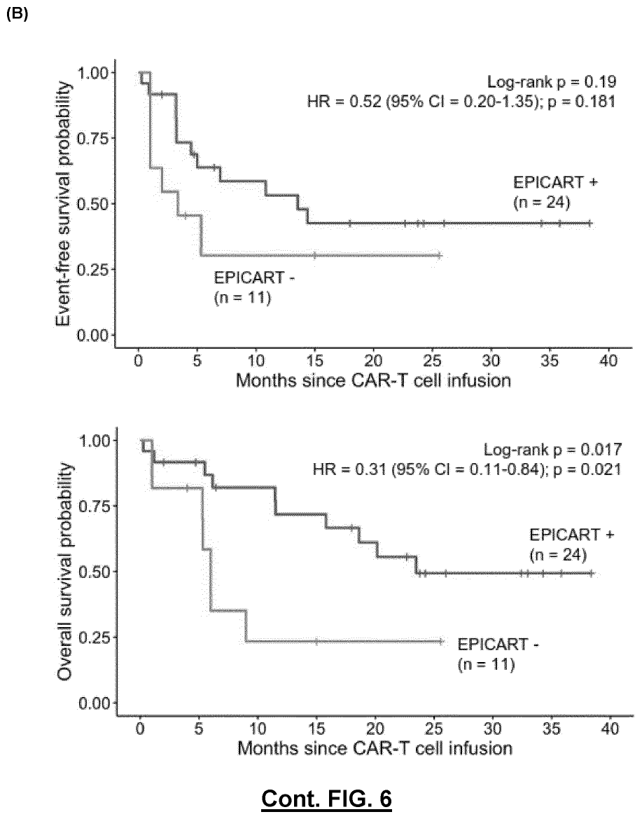

Having characterized the EPICART18 signature as being a predictor of CR, EFS and OS in the discovery cohort of B-cell malignancies treated with CART19, it was interrogated whether the identified DNA methylation landscape could also distinguish clinical outcome in the validation cohort (Table C). From a clinical standpoint, CR was associated with enhanced EFS and improved OS in the validation set (FIG. 6 (A)).

Importantly, EPICART18 signature predicted CR to CAR-T cell therapy with 83% accuracy (95% CI=66-93; Kappa=0.6), 88% sensitivity and 73% specificity in the validation cohort. It was further evaluated the model performance using the ROC curve obtaining an AUC value of 0.8. The use of the EPICART18 signature in the supervised hierarchical clustering for the validation cohort of CAR-T cases also distinguished CR or non-CR (Fisher's exact test, P=0.0001). Remarkably, the EPICART18-positive signature was associated with improved OS in the validation cohort (HR=0.31, 95% CI=0.112-0.837, P=0.021; log-rank P=0.017) (FIG. 6 (B)). It was also found a non-significant trend between the EPICART18-positive signature and EFS (HR=0.52, 95% CI=0.204-1.349, P=0.181; log-rank P=0.19) (FIG. 6 (B)).

Finally, for the entire cohort, CR was associated with EFS and OS. The EPICART18 signature in the supervised hierarchical clustering for the complete set of available cases (discovery+validation, n=114) also classified patients as those exhibiting CR or non-CR (Fisher's exact test, P=3.5e-15). Importantly, in the entire cohort, EPICART18-positive signature was associated with improved EFS and OS. The HRs and p-values for EFS and OS obtained from each cohort are summarized in next Tables F1 to F4.

TABLE F1
Event free survival (EFS) for CR vs. non-CR

| | Complete response (CR) vs. non-CR (PR/SD/PD) | | | |
|---|---|---|---|---|
| | Log-rank | Hazard | 95% confidence | HR |
| | p-value | ratio (HR) | interval (CI) | p-value |
| Discovery | 7.20E−12 | 0.12 | 0.06-0.24 | 7.50E−10 |
| Cohort | | | | |
| Validation | 0.002 | 0.24 | 0.09-0.62 | 0.003 |
| cohort | | | | |
| Entire cohort | 4.10E−14 | 0.15 | 0.09-0.26 | 4.50E−12 |

TABLE F2
Event free survival (EFS) for EPICART18+ vs EPICART18−

| | EPICART18+ vs. EPICART18− | | | |
|---|---|---|---|---|
| | Log-rank | Hazard | 95% confidence | HR |
| | p-value | ratio (HR) | interval (CI) | p-value |
| Discovery | 0.003 | 0.36 | 0.19-0.70 | 0.002 |
| cohort | | | | |
| Validation | 0.19 | 0.52 | 0.20-1.35 | 0.181 |
| cohort | | | | |
| Entire cohort | 0.003 | 0.43 | 0.26-0.74 | 0.002 |

TABLE F3
Overall survival (OS) for CR vs. non-CR

| | Complete response (CR) vs. non-CR (PR/SD/PD) | | | |
|---|---|---|---|---|
| | Log-rank | Hazard | 95% confidence | HR |
| | p-value | ratio (HR) | interval (CI) | p-value |
| Discovery | 9.90E−07 | 0.18 | 0.09-0.39 | 9.60E−06 |
| cohort | | | | |
| Validation | 3.10E−05 | 0.11 | 0.04-0.37 | 0.0003 |
| cohort | | | | |
| Entire cohort | 5.10E−10 | 0.18 | 0.10-0.32 | 1.90E−08 |

TABLE F4
Overall survival (OS) for EPICART18+ vs EPICART18−

| | EPICART18+ vs. EPICART18− | | | |
|---|---|---|---|---|
| | Log-rank | Hazard | 95% confidence | HR |
| | p-value | ratio (HR) | interval (CI) | p-value |
| Discovery | 0.042 | 0.45 | 0.20-0.99 | 0.047 |
| cohort | | | | |
| Validation | 0.017 | 0.31 | 0.11-0.84 | 0.021 |
| cohort | | | | |
| Entire cohort | 0.003 | 0.39 | 0.21-0.74 | 0.003 |

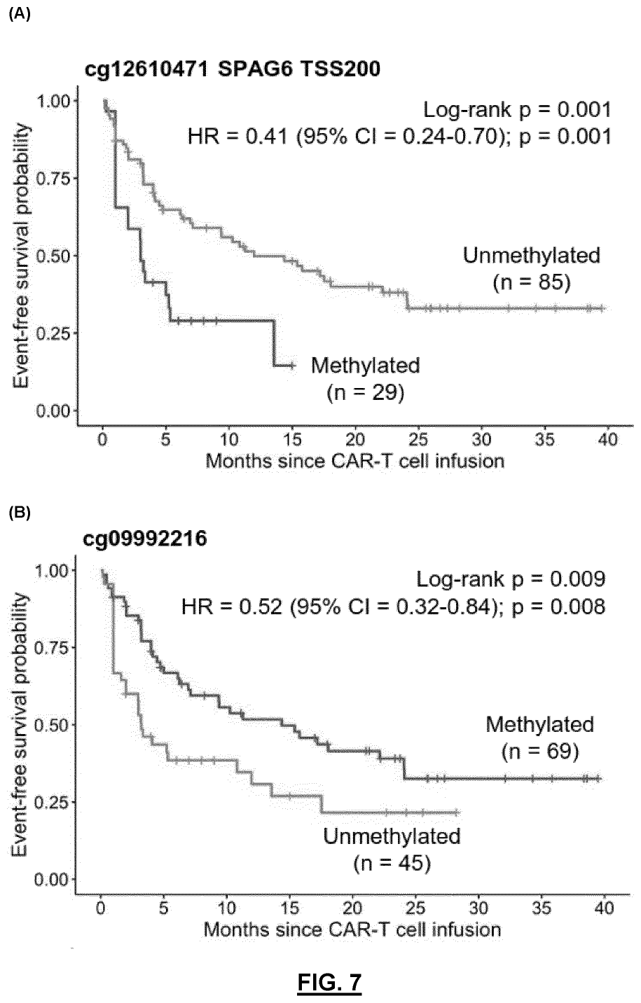

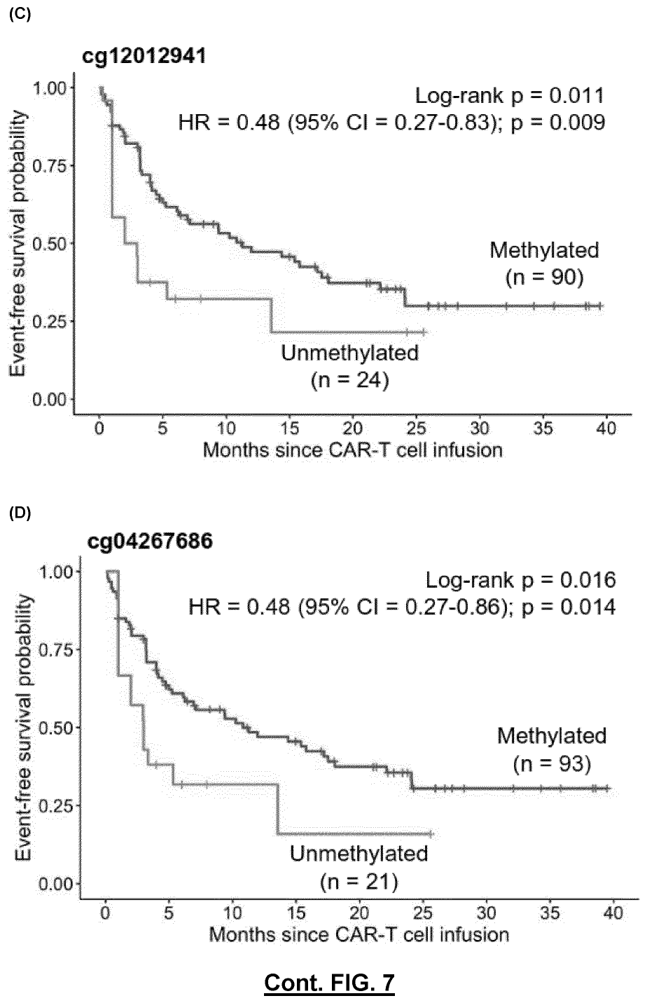

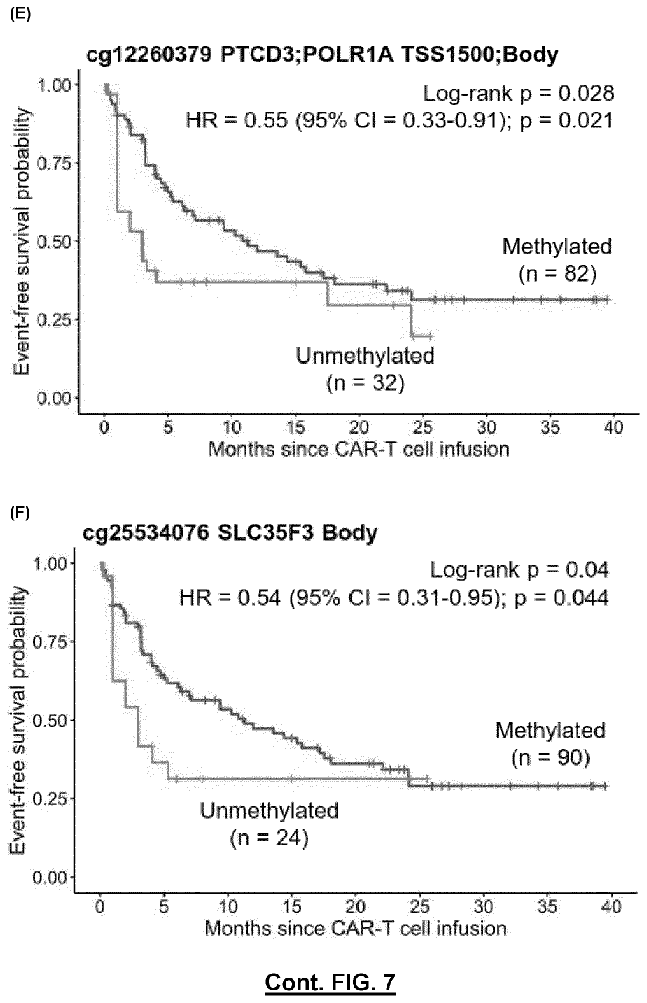

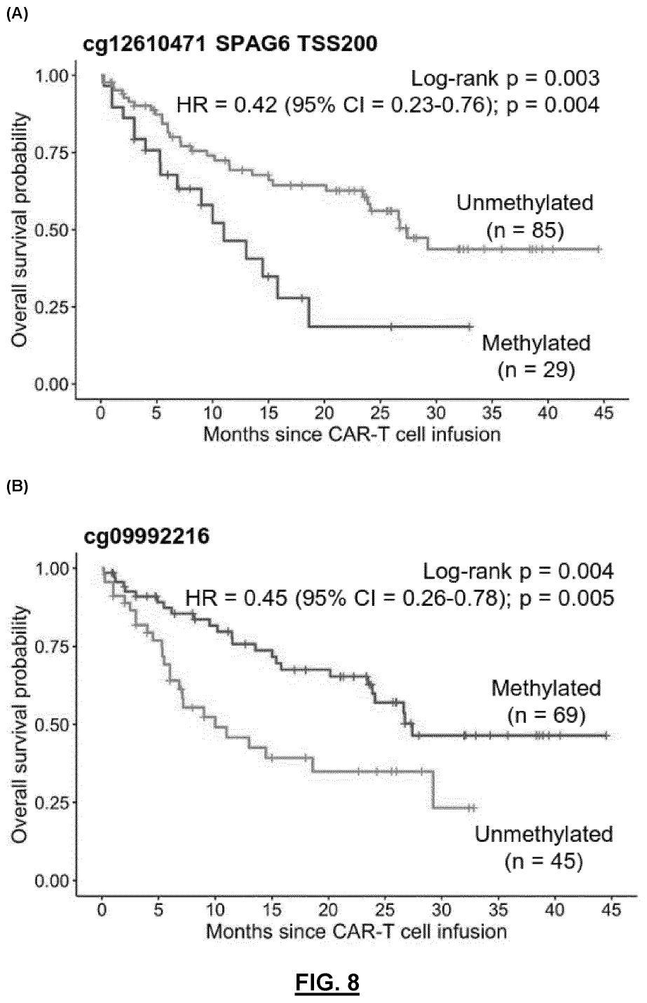

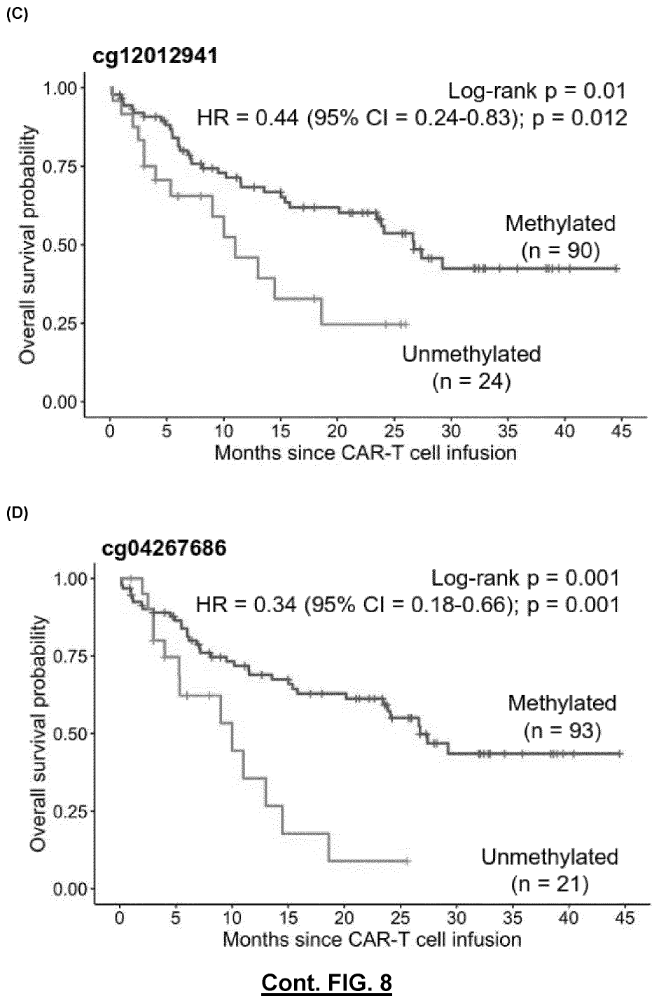

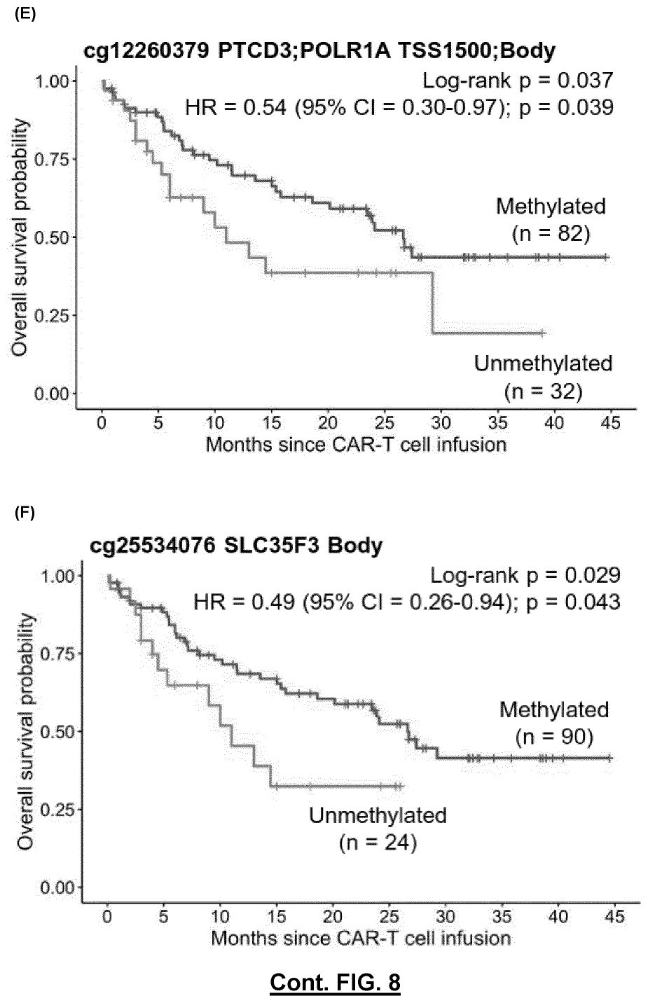

To identify a smaller set of biomarkers that could simplify the analysis, it was found six epigenomic loci from the EPICART18 signature that, analyzed alone, were also associated with improved EFS and OS. These CpG sites are summarized in Table A, illustrated above in this description, and the corresponding Kaplan-Meier curves for EFS and OS are shown in FIG. 7 (A-F) and FIG. 8 (A-F), respectively.

As a summary and discussion of the results in Example 2, corroborating data of Example 1 with a small discovery cohort, corroborates that the epigenetic profiling in CAR19-transduced T-lymphocytes provides a consistent readout associated with clinical outcome. The findings provide evidence that the intrinsic molecular features of the pre-infusion cells determine the success of the adoptive cell therapy. In this regard, the global RNA expression patterns of the pre-infused T-cell differs between CR and non-CR patients, an observation added to the impact on outcome of the CAR integration site. All these findings support that the “fitness” of the pre-infused CART19 cells contributes to treatment effectiveness. In this regard, CART19 cell products that harbor particular T-cell subsets are more clinically effective. Differences in the conditions of the manufacturing process from commercially available treatments, and the unique functional background of the transduced T-cells of each patient, can modify the “omics” landscape of pre-infused cells, directly affecting their activity. Importantly, it has been recently reported that epigenetic remodeling can restore functionality in exhausted CAR-T cells (Weber E W, Parker K R, Sotillo E, et al. Transient rest restores functionality in exhausted CAR-T cells through epigenetic remodeling. Science. 2021; 372(6537):eaba1786), further supporting the impact of these changes.

These results strengthen the notion that the molecular profiles of the cells used in adoptive cell therapy is of great value for determining treatment success. This approach has also been proposed for immune checkpoint inhibitors in the prior art (Duruisseaux M, Martínez-Cardús A, Calleja-Cervantes M E, et al. Epigenetic prediction of response to anti-PD-1 treatment in non-small-cell lung cancer: a multicentre, retrospective analysis. Lancet Respir Med. 2018; 6(10):771-781.). Thus, biomarkers of the efficacy of adoptive cell therapy, similar to those cited here, and the DNA methylation markers proposed by this invention, almost certainly await discovery. Two examples highlight the potential of studies in this area.

Overall, the DNA methylation landscape of pre-infusion CART19 cells can predict which patients with B-cell malignancy will gain a clinical benefit. Importantly for its proposed clinical use, the best of the candidate sites identified within the epigenomic signatures disclosed in this invention could also be assessed, as indicated, using single PCR-based assays. In this regard, assessing the epigenetic profile of the CAR19-transduced pre-infused T-cells could help solve the unmet medical need to identify the patients who would benefit the most of CAR T-cell therapy.

TABLE S1
CpG sites that were distinctly methylated
in CAR-positive and CAR-negative T-cells

| | Chromosomal | |
|---|---|---|
| Probe ID | position (hg19) | Associated gene |
| cg16269199 | chr1: 10850 | DDX11L1 |
| cg09789536 | chr1: 896226 | KLHL17 |
| cg20594982 | chr1: 976707 | AGRN |
| cg07407159 | chr1: 979484 | AGRN |
| cg09315878 | chr1: 1152580 | SDF4 |
| cg22220310 | chr1: 1168541 | SDF4, B3GALT6 |
| cg19639479 | chr1: 1374669 | VWA1 |
| cg02769781 | chr1: 1713676 | Not described |
| cg11835265 | chr1: 2011913 | PRKCZ |
| cg17286074 | chr1: 3315497 | PRDM16 |
| cg25469923 | chr1: 4087182 | Not described |
| cg11201308 | chr1: 5151290 | Not described |
| cg00777636 | chr1: 6446216 | ACOT7 |
| cg09272923 | chr1: 7730096 | CAMTA1 |
| cg07180616 | chr1: 8823146 | RERE |
| cg18264158 | chr1: 9262713 | Not described |
| cg26363759 | chr1: 9377984 | SPSB1 |
| cg02096887 | chr1: 10699831 | CASZ1 |
| cg26522708 | chr1: 10839450 | CASZ1 |
| cg07552723 | chr1: 14092249 | PRDM2 |
| cg24452347 | chr1: 24229300 | CNR2 |
| cg26404511 | chr1: 24229575 | CNR2 |
| cg27424995 | chr1: 27902555 | AHDC1 |
| cg07343000 | chr1: 28969881 | TAF12 |
| cg02771649 | chr1: 31474920 | PUM1 |
| cg02678971 | chr1: 32042625 | TINAGL1 |
| cg01938422 | chr1: 35659480 | SFPQ |
| cg14962509 | chr1: 36039655 | TFAP2E |
| cg13423759 | chr1: 37937403 | Not described |
| cg04408387 | chr1: 38183556 | EPHA10 |
| cg06959800 | chr1: 38233563 | Not described |
| cg16191204 | chr1: 39456828 | AKIRIN1 |
| cg11676353 | chr1: 39548001 | MACF1 |
| cg16730386 | chr1: 46269394 | MAST2 |
| cg10784298 | chr1: 46269397 | MAST2 |
| cg01160877 | chr1: 47689941 | TAL1 |
| cg05174922 | chr1: 53291756 | ZYG11B |
| cg24550149 | chr1: 55246954 | TTC22 |
| cg11949335 | chr1: 55247408 | TTC22 |
| cg21561370 | chr1: 62660428 | L1TD1 |
| cg22171055 | chr1: 62905816 | USP1 |
| cg05079074 | chr1: 63212615 | Not described |
| cg10626744 | chr1: 63783466 | Not described |
| cg14651192 | chr1: 63999206 | EFCAB7 |
| cg08718880 | chr1: 74970626 | TNNI3K |
| cg27534367 | chr1: 77532151 | Not described |
| cg01999380 | chr1: 83373070 | Not described |
| cg00606914 | chr1: 90229063 | Not described |
| cg23646383 | chr1: 94054497 | BCAR3 |
| cg02978297 | chr1: 94057587 | BCAR3 |
| cg22841328 | chr1: 95585272 | TMEM56, TMEM56RWDD3 |
| cg00182921 | chr1: 100466941 | SLC35A3 |
| cg06861375 | chr1: 120166167 | ZNF697 |
| cg18561676 | chr1: 148855262 | Not described |
| cg01990592 | chr1: 151105438 | SEMA6C |
| cg14204738 | chr1: 151799266 | RORC |
| cg12600901 | chr1: 154166440 | MIR190B |
| cg07140482 | chr1: 154956222 | FLAD1 |
| cg06359077 | chr1: 156838625 | NTRK1 |
| cg06353725 | chr1: 162036019 | Not described |
| cg22329438 | chr1: 166957790 | MAEL |
| cg03839297 | chr1: 168461317 | LOC101928565 |
| cg09168501 | chr1: 170114243 | METTL11B |
| cg00263210 | chr1: 173838039 | SNORD74, GAS5, ZBTB37 |
| cg24562221 | chr1: 174975532 | CACYBP |
| cg25366573 | chr1: 180810642 | XPR1 |
| cg20741134 | chr1: 181382639 | Not described |
| cg14957855 | chr1: 185234541 | SWT1 |
| cg12012941 | chr1: 188676237 | Not described |
| cg24365464 | chr1: 190448126 | FAM5C |
| cg09554300 | chr1: 201123894 | TMEM9 |
| cg06286642 | chr1: 203595825 | ATP2B4 |
| cg14333970 | chr1: 205689503 | NUCKS1 |
| cg17893351 | chr1: 206845160 | Not described |
| cg21575235 | chr1: 212505404 | PPP2R5A |
| cg00063699 | chr1: 214624242 | PTPN14 |
| cg06645242 | chr1: 218515560 | Not described |
| cg25317664 | chr1: 220101962 | SLC30A10 |
| cg04458195 | chr1: 220414164 | RAB3GAP2 |
| cg07883844 | chr1: 226111912 | PYCR2 |
| cg11401257 | chr1: 226926945 | ITPKB |
| cg22163199 | chr1: 228297864 | MRPL55 |
| cg06796869 | chr1: 228471009 | OBSCN |
| cg00817100 | chr1: 229543254 | Not described |
| cg00572889 | chr1: 230493461 | PGBD5 |
| cg05820241 | chr1: 232442534 | Not described |
| cg25534076 | chr1: 234087867 | SLC35F3 |
| cg13646833 | chr1: 234981443 | Not described |
| cg07953201 | chr1: 245851802 | KIF26B |
| cg06248935 | chr1: 247040446 | AHCTF1 |
| cg02713706 | chr1: 247681749 | GCSAML |
| cg20771596 | chr1: 247681766 | GCSAML |
| cg11166453 | chr1: 247681781 | Not described |
| cg12754571 | chr1: 247694271 | LOC148824, OR2C3 |
| cg12758973 | chr1: 247694275 | LOC148824, OR2C3 |
| cg20506745 | chr2: 2581285 | Not described |
| cg10549986 | chr2: 7018153 | RSAD2 |
| cg14780466 | chr2: 20870812 | GDF7 |
| cg17779336 | chr2: 24299434 | SF3B14 |
| cg24089715 | chr2: 25017714 | PTRHD1, CENPO |
| cg03859915 | chr2: 28112817 | BRE, RBKS, LOC100302650 |
| cg03983703 | chr2: 32585694 | BIRC6 |
| cg26016587 | chr2: 33032061 | TTC27 |
| cg05238843 | chr2: 38975790 | SRSF7 |
| cg01746088 | chr2: 38978194 | SFRS7 |
| cg15889012 | chr2: 44059266 | ABCG5 |
| cg03593578 | chr2: 45028225 | Not described |
| cg18682873 | chr2: 46769828 | RHOQ |
| cg18190286 | chr2: 54067920 | GPR75ASB3 |
| cg21893107 | chr2: 70521592 | SNRPG |
| cg12463797 | chr2: 73087042 | Not described |
| cg06700871 | chr2: 84686330 | SUCLG1 |
| cg19627006 | chr2: 85637673 | CAPG |
| cg12260379 | chr2: 86332162 | PTCD3, POLR1A |
| cg07939752 | chr2: 87082884 | CD8B |
| cg18014437 | chr2: 95719069 | MAL |
| cg20051635 | chr2: 98340909 | ZAP70 |
| cg14131536 | chr2: 98340921 | ZAP70 |
| cg00120880 | chr2: 106288902 | Not described |
| cg01446203 | chr2: 119613126 | Not described |
| cg00421221 | chr2: 121106091 | INHBB |
| cg17511575 | chr2: 122144477 | CLASP1 |
| cg15093938 | chr2: 129147579 | Not described |
| cg01925114 | chr2: 130517725 | Not described |
| cg01311063 | chr2: 131058184 | Not described |
| cg23123222 | chr2: 131792521 | ARHGEF4 |
| cg14161159 | chr2: 132481826 | C2orf27A |
| cg26247107 | chr2: 143580173 | Not described |
| cg00551617 | chr2: 152152581 | Not described |
| cg18818774 | chr2: 153515945 | PRPF40A |
| cg13546658 | chr2: 162164472 | PSMD14 |
| cg24367840 | chr2: 162197869 | PSMD14 |
| cg17137077 | chr2: 162197875 | PSMD14 |
| cg27503015 | chr2: 162270453 | Not described |
| cg19318707 | chr2: 162847258 | Not described |
| cg07855447 | chr2: 168401353 | Not described |
| cg16202181 | chr2: 170870436 | UBR3 |
| cg16955800 | chr2: 183981465 | NUP35 |
| cg10258931 | chr2: 203639072 | ICA1L |
| cg17626178 | chr2: 205410273 | PARD3B |
| cg18626478 | chr2: 205591269 | PARD3B |
| cg19988790 | chr2: 209786106 | Not described |
| cg19922558 | chr2: 211950311 | Not described |
| cg14538944 | chr2: 218340518 | DIRC3 |
| cg04688351 | chr2: 223154140 | PAX3 |
| cg05059613 | chr2: 224821947 | MRPL44 |
| cg14853771 | chr2: 231921295 | PSMD1 |
| cg08251704 | chr2: 234296312 | DGKD |
| cg12552337 | chr2: 235988130 | Not described |
| cg10940914 | chr2: 236290930 | Not described |
| cg12269677 | chr2: 236801884 | AGAP1 |
| cg21300993 | chr2: 237123218 | ASB18 |
| cg11999858 | chr2: 237414073 | IQCA1 |
| cg18173230 | chr2: 238207644 | Not described |
| cg07797518 | chr2: 238536051 | LRRFIP1 |
| cg07018857 | chr2: 239834442 | Not described |
| cg10067182 | chr2: 239974554 | HDAC4 |
| cg23631133 | chr2: 240568824 | Not described |
| cg05439349 | chr3: 5032114 | Not described |
| cg21477613 | chr3: 10385984 | ATP2B2 |
| cg24691167 | chr3: 10749371 | Not described |
| cg01320920 | chr3: 12597236 | MKRN2 |
| cg12448747 | chr3: 12898045 | Not described |
| cg18696115 | chr3: 14263394 | Not described |
| cg00329101 | chr3: 14338921 | Not described |
| cg07573937 | chr3: 14465629 | SLC6A6 |
| cg04804472 | chr3: 21267468 | Not described |
| cg09463922 | chr3: 31657140 | STT3B |
| cg14481081 | chr3: 32558117 | Not described |
| cg23854042 | chr3: 43737088 | ABHD5 |
| cg01559446 | chr3: 44596178 | ZNF167 |
| cg22502448 | chr3: 44622831 | ZNF167 |
| cg14416782 | chr3: 45505849 | LARS2 |
| cg11733272 | chr3: 46940439 | PTH1R |
| cg02222844 | chr3: 48694451 | CELSR3 |
| cg01079779 | chr3: 57176708 | IL17RD |
| cg24036280 | chr3: 62365402 | Not described |
| cg22117381 | chr3: 65840792 | MAGI1 |
| cg06217323 | chr3: 75445502 | Not described |
| cg10728960 | chr3: 78079111 | Not described |
| cg13790797 | chr3: 99595841 | FILIP1L, MIR548G, CMSS1 |
| cg20824237 | chr3: 99979579 | TBC1D23 |
| cg13024275 | chr3: 101579557 | NFKBIZ |
| cg24214168 | chr3: 111259879 | CD96 |
| cg14511498 | chr3: 115500667 | Not described |
| cg00920424 | chr3: 127172519 | Not described |
| cg11853366 | chr3: 127865242 | Not described |
| cg11277477 | chr3: 128831705 | RAB43, ISY1RAB43 |
| cg10602180 | chr3: 137729133 | CLDN18 |
| cg10521230 | chr3: 137798210 | DZIP1L |
| cg16106292 | chr3: 145787428 | PLOD2 |
| cg00842299 | chr3: 147129278 | ZIC1 |
| cg02535198 | chr3: 148621216 | Not described |
| cg17278207 | chr3: 148768046 | HLTF |
| cg08242158 | chr3: 151559625 | AADACL2AS1 |
| cg15277914 | chr3: 154042611 | DHX36 |
| cg00992239 | chr3: 154797349 | MME |
| cg03139244 | chr3: 168762673 | Not described |
| cg23313725 | chr3: 172259279 | Not described |
| cg03024690 | chr3: 172268675 | Not described |
| cg15672986 | chr3: 175465989 | NAALADL2 |
| cg13143743 | chr3: 177570694 | Not described |
| cg02524983 | chr3: 188012918 | LPP |
| cg27331225 | chr3: 193852938 | HES1 |
| cg10793234 | chr3: 194062061 | CPN2 |
| cg07393816 | chr3: 196595274 | SENP5 |
| cg00009352 | chr3: 197712358 | LMLN |
| cg09763814 | chr4: 680234 | MFSD7 |
| cg05294497 | chr4: 1342808 | KIAA1530 |
| cg25987564 | chr4: 6010075 | Not described |
| cg25970575 | chr4: 6010164 | Not described |
| cg23892390 | chr4: 8603012 | CPZ |
| cg10865436 | chr4: 11911016 | Not described |
| cg11793663 | chr4: 20884476 | KCNIP4 |
| cg01453391 | chr4: 22636392 | Not described |
| cg06563796 | chr4: 24825568 | CCDC149 |
| cg09490880 | chr4: 24914970 | CCDC149 |
| cg26448612 | chr4: 25348745 | ZCCHC4 |
| cg13732582 | chr4: 39448975 | KLB |
| cg04285136 | chr4: 39910427 | PDS5A |
| cg11641097 | chr4: 42054953 | SLC30A9 |
| cg02339197 | chr4: 47984349 | CNGA1, LOC101927157 |
| cg02808656 | chr4: 48385504 | SLAIN2 |
| cg01389693 | chr4: 70505439 | UGT2A2, UGT2A1 |
| cg25363886 | chr4: 71705620 | GRSF1 |
| cg18804985 | chr4: 74965226 | CXCL2 |
| cg12744223 | chr4: 76752176 | Not described |
| cg22902161 | chr4: 94683967 | GRID2 |
| cg06689619 | chr4: 99935464 | METAP1 |
| cg26651837 | chr4: 104695090 | Not described |
| cg15955354 | chr4: 125174087 | Not described |
| cg02075590 | chr4: 125607552 | ANKRD50 |
| cg00120464 | chr4: 146842191 | ZNF827 |
| cg10781413 | chr4: 153656592 | Not described |
| cg15065144 | chr4: 157978856 | Not described |
| cg06885374 | chr4: 159131626 | TMEM144 |
| cg10962056 | chr4: 165034710 | MARCH1 |
| cg09932305 | chr4: 170526287 | NEK1 |
| cg09469540 | chr4: 175462076 | Not described |
| cg19759671 | chr4: 183063459 | MGC45800 |
| cg22051345 | chr4: 183721183 | ODZ3 |
| cg00306510 | chr4: 187003000 | TLR3 |
| cg11264959 | chr5: 458948 | EXOC3 |
| cg26427908 | chr5: 1225074 | SLC6A19, SLC6A18 |
| cg17026582 | chr5: 1298573 | Not described |
| cg05614378 | chr5: 7886711 | MTRR |
| cg14866978 | chr5: 14098648 | Not described |
| cg26362078 | chr5: 27036352 | CDH9 |
| cg25996586 | chr5: 32786671 | NPR3 |
| cg23986315 | chr5: 33473240 | Not described |
| cg24595208 | chr5: 34496537 | Not described |
| cg06533086 | chr5: 39139105 | FYB |
| cg10487770 | chr5: 57879443 | RAB3C |
| cg11258089 | chr5: 59189791 | PDE4D |
| cg02306844 | chr5: 64481118 | ADAMTS6 |
| cg18975733 | chr5: 73852639 | Not described |
| cg09605533 | chr5: 79183067 | Not described |
| cg00795341 | chr5: 79330929 | THBS4 |
| cg18467053 | chr5: 80160666 | MSH3 |
| cg14311008 | chr5: 82060880 | Not described |
| cg21229536 | chr5: 82373428 | XRCC4, TMEM167A |
| cg23917249 | chr5: 86619808 | RASA1 |
| cg11173822 | chr5: 101925501 | Not described |
| cg22562498 | chr5: 111095202 | C5orf13 |
| cg06003475 | chr5: 115890768 | SEMA6A |
| cg19893178 | chr5: 118692011 | TNFAIP8 |
| cg21441748 | chr5: 121517083 | LOC100505841 |
| cg25780348 | chr5: 125936421 | PHAX |
| cg06943925 | chr5: 132145434 | Not described |
| cg02142990 | chr5: 137915194 | Not described |
| cg22235387 | chr5: 138620501 | MATR3 |
| cg12193929 | chr5: 140237485 | PCDHA6, PCDHA2, |
| | | PCDH1, PCDHA9, |
| | | PCDHA7, PCDHA, |
| | | PCDHA3, PCDHA4, |
| | | PCDHA, PCDHA8 |
| cg01904727 | chr5: 145484705 | PLAC8L1 |
| cg08734395 | chr5: 163086064 | Not described |
| cg15913291 | chr5: 171433817 | FBXW11 |
| cg15998629 | chr5: 172000858 | Not described |
| cg24667575 | chr5: 172097064 | NEURL1B |
| cg00327072 | chr5: 172110477 | NEURL1B |
| cg15835735 | chr5: 173135031 | LINC01484 |
| cg04542001 | chr5: 177926387 | COL23A1 |
| cg09444392 | chr5: 179781245 | GFPT2 |
| cg25606201 | chr5: 180614858 | Not described |
| cg15253304 | chr6: 209809 | Not described |
| cg19137726 | chr6: 4082055 | C6orf201 |
| cg06501113 | chr6: 6534839 | LOC285780 |
| cg09367268 | chr6: 6643814 | LY86 |
| cg24336989 | chr6: 10385903 | Not described |
| cg22836400 | chr6: 10415636 | TFAP2A |
| cg02280355 | chr6: 13680154 | RANBP9 |
| cg15274294 | chr6: 14884128 | Not described |
| cg03625260 | chr6: 17102391 | Not described |
| cg05312353 | chr6: 17102678 | Not described |
| cg09728223 | chr6: 17619088 | NUP153 |
| cg05042653 | chr6: 21586259 | Not described |
| cg06213964 | chr6: 21665905 | FLJ22536 |
| cg02888247 | chr6: 24719235 | C6orf62 |
| cg26314781 | chr6: 26365485 | BTN3A2 |
| cg12437199 | chr6: 28551719 | SCAND3 |
| cg12934884 | chr6: 28558070 | Not described |
| cg10933494 | chr6: 28983143 | Not described |
| cg18976822 | chr6: 30524797 | PRR3, GNL1 |
| cg21055528 | chr6: 31549631 | LTB |
| cg13887029 | chr6: 31746795 | VARS |
| cg21087321 | chr6: 31850465 | EHMT2 |
| cg00872984 | chr6: 32063991 | TNXB |
| cg00525277 | chr6: 32064239 | TNXB |
| cg27338898 | chr6: 32163411 | NOTCH4, GPSM3 |
| cg01738650 | chr6: 32223076 | Not described |
| cg25924085 | chr6: 32324878 | C6orf10 |
| cg22696073 | chr6: 32939982 | BRD2 |
| cg01140143 | chr6: 33039396 | HLADPA1 |
| cg14043298 | chr6: 33136297 | COL11A2 |
| cg18849417 | chr6: 33145168 | COL11A2 |
| cg23581489 | chr6: 33164210 | RXRB |
| cg01536956 | chr6: 33165577 | RXRB |
| cg02775469 | chr6: 33181031 | Not described |
| cg02863594 | chr6: 33280199 | TAPBP |
| cg14999947 | chr6: 33422918 | ZBTB9 |
| cg18047961 | chr6: 33878466 | Not described |
| cg25847633 | chr6: 35744230 | C6orf126 |
| cg10664338 | chr6: 35996736 | MAPK14 |
| cg02655365 | chr6: 36507858 | STK38 |
| cg26848940 | chr6: 37787530 | ZFAND3 |
| cg14829325 | chr6: 38743257 | DNAH8 |
| cg06061086 | chr6: 41499640 | Not described |
| cg16538289 | chr6: 41703332 | TFEB |
| cg11414345 | chr6: 42106434 | C6orf132 |
| cg09221605 | chr6: 43386386 | Not described |
| cg16694973 | chr6: 45803647 | Not described |
| cg21756828 | chr6: 45984502 | CLIC5 |
| cg04652496 | chr6: 46294097 | RCAN2 |
| cg22480773 | chr6: 49466589 | GLYATL3 |
| cg01106989 | chr6: 52858459 | GSTA4 |
| cg21763653 | chr6: 52945383 | FBXO9 |
| cg05920635 | chr6: 55039622 | HCRTR2 |
| cg06015119 | chr6: 56911219 | KIAA1586 |
| cg00652171 | chr6: 67920750 | Not described |
| cg04319895 | chr6: 72955528 | RIMS1 |
| cg26302094 | chr6: 73973103 | KHDC1 |
| cg13663057 | chr6: 78002283 | Not described |
| cg13554177 | chr6: 79780164 | PHIP |
| cg04181542 | chr6: 100584390 | Not described |
| cg26499055 | chr6: 100909238 | SIM1 |
| cg04267686 | chr6: 105907265 | Not described |
| cg19344103 | chr6: 108583071 | SNX3 |
| cg19196401 | chr6: 110721138 | DDO |
| cg23579533 | chr6: 116988869 | ZUFSP |
| cg15806890 | chr6: 118868338 | PLN, CEP85L |
| cg25571136 | chr6: 127612751 | ECHDC1 |
| cg23281432 | chr6: 130554730 | Not described |
| cg22244427 | chr6: 132281492 | Not described |
| cg18136963 | chr6: 139013146 | Not described |
| cg18736889 | chr6: 140203833 | Not described |
| cg02589130 | chr6: 143379277 | AIG1 |
| cg07077459 | chr6: 144329052 | HYMAI, PLAGL1 |
| cg22378065 | chr6: 144329172 | HYMAI, PLAGL1 |
| cg22352234 | chr6: 144329382 | PLAGL1, HYMAI |
| cg00702231 | chr6: 144329473 | PLAGL1, HYMAI |
| cg12757684 | chr6: 144329485 | PLAGL1, HYMAI |
| cg02279224 | chr6: 144386160 | PLAGL1 |
| cg09318120 | chr6: 149638382 | MAP3K7IP2 |
| cg05593653 | chr6: 152701495 | SYNE1AS1, SYNE1 |
| cg01412668 | chr6: 155544957 | TIAM2 |
| cg17511936 | chr6: 155566057 | TIAM2 |
| cg02018347 | chr6: 162857617 | PARK2 |
| cg00139234 | chr6: 163922286 | QKI |
| cg16149820 | chr6: 164251509 | Not described |
| cg14497223 | chr6: 168534096 | Not described |
| cg17529386 | chr6: 170452270 | Not described |
| cg19848529 | chr6: 170483067 | Not described |
| cg22099441 | chr6: 170531660 | Not described |
| cg25929664 | chr7: 220654 | FAM20C |
| cg02240103 | chr7: 880002 | UNC84A |
| cg01966674 | chr7: 908060 | UNC84A |
| cg05486810 | chr7: 1057463 | C7orf50 |
| cg01140416 | chr7: 1293629 | Not described |
| cg01938825 | chr7: 1563708 | Not described |
| cg04326741 | chr7: 1937120 | MAD1L1 |
| cg12395958 | chr7: 1973573 | MAD1L1 |
| cg20308351 | chr7: 3067980 | CARD11 |
| cg13589218 | chr7: 4783964 | FOXK1 |
| cg06287775 | chr7: 4784174 | FOXK1 |
| cg10406205 | chr7: 4837835 | Not described |
| cg09829164 | chr7: 4918779 | RADIL |
| cg15262352 | chr7: 5013468 | RNF216L |
| cg00730085 | chr7: 5115365 | Not described |
| cg12838902 | chr7: 5322586 | SLC29A4 |
| cg19261626 | chr7: 5739735 | RNF216 |
| cg26834624 | chr7: 6662329 | ZNF853 |
| cg15784006 | chr7: 6692445 | Not described |
| cg20964328 | chr7: 11013816 | PHF14 |
| cg27334919 | chr7: 19158378 | TWIST1 |
| cg16448423 | chr7: 21051054 | LINC01162 |
| cg13069322 | chr7: 22047028 | Not described |
| cg11990443 | chr7: 23454952 | IGF2BP3 |
| cg18683539 | chr7: 25994421 | Not described |
| cg19521279 | chr7: 27144595 | Not described |
| cg23884241 | chr7: 27169957 | HOXA4 |
| cg16651126 | chr7: 27170552 | HOXA4 |
| cg11015251 | chr7: 27170554 | HOXA4 |
| cg22158650 | chr7: 27291388 | Not described |
| cg03453916 | chr7: 31174468 | Not described |
| cg16380877 | chr7: 31447845 | Not described |
| cg03673694 | chr7: 37025706 | ELMO1 |
| cg11830489 | chr7: 39045208 | POU6F2 |
| cg19915007 | chr7: 41189312 | Not described |
| cg26309511 | chr7: 48887640 | Not described |
| cg16207528 | chr7: 48964051 | CDC14C |
| cg12903171 | chr7: 50850564 | GRB10 |
| cg00945507 | chr7: 54827677 | SEC61G |
| cg22902969 | chr7: 56131795 | SUMF2 |
| cg19627238 | chr7: 63641030 | Not described |
| cg09265173 | chr7: 72972201 | BCL7B |
| cg09865216 | chr7: 73323323 | Not described |
| cg25712005 | chr7: 73443113 | ELN |
| cg00111463 | chr7: 77137824 | Not described |
| cg06551493 | chr7: 77166702 | PTPN12 |
| cg17578341 | chr7: 81420133 | Not described |
| cg24755033 | chr7: 82789373 | PCLO |
| cg06258179 | chr7: 92463261 | CDK6 |
| cg23690528 | chr7: 94285270 | SGCE, PEG10 |
| cg25430192 | chr7: 96643508 | DLX6AS |
| cg08302003 | chr7: 100435192 | Not described |
| cg14823389 | chr7: 101398152 | Not described |
| cg00320861 | chr7: 103075709 | SLC26A5 |
| cg02934649 | chr7: 103790671 | ORC5 |
| cg21884374 | chr7: 107807954 | NRCAM |
| cg24764243 | chr7: 107968775 | NRCAM |
| cg15108650 | chr7: 112420018 | TMEM168 |
| cg20993361 | chr7: 116503444 | CAPZA2 |
| cg16772591 | chr7: 120373072 | KCND2 |
| cg03744115 | chr7: 123242767 | NDUFA5, ASB15 |
| cg10316026 | chr7: 124565947 | POT1 |
| cg20186636 | chr7: 128470230 | FLNC |
| cg06314333 | chr7: 129425532 | Not described |
| cg16823958 | chr7: 130131869 | MEST, MESTIT1 |
| cg09080913 | chr7: 130131887 | MEST, MESTIT1 |
| cg13104298 | chr7: 130131905 | MEST, MESTIT1 |
| cg20050761 | chr7: 130131923 | MEST, MESTIT1 |
| cg05556276 | chr7: 130132161 | MEST, MESTIT1 |
| cg17580798 | chr7: 130132199 | MEST, MESTIT1 |
| cg01784351 | chr7: 130132265 | MEST, MESTIT1 |
| cg27589003 | chr7: 130132286 | MEST, MESTIT1 |
| cg17366173 | chr7: 133614443 | EXOC4 |
| cg16550959 | chr7: 133765461 | Not described |
| cg09293560 | chr7: 150068240 | REPIN1 |
| cg15188808 | chr7: 150094161 | ZNF775 |
| cg00852675 | chr7: 150105086 | LOC728743 |
| cg16994041 | chr7: 151503269 | PRKAG2 |
| cg24111710 | chr7: 152063974 | MLL3 |
| cg07227743 | chr7: 154474113 | DPP6 |
| cg13028819 | chr7: 156157689 | Not described |
| cg26640683 | chr7: 156297193 | Not described |
| cg23299919 | chr7: 157406096 | PTPRN2 |
| cg02601082 | chr7: 158612297 | ESYT2 |
| cg13817083 | chr7: 159026609 | Not described |
| cg23109344 | chr8: 496440 | C8orf42 |
| cg14755254 | chr8: 637813 | ERICH1 |
| cg20916500 | chr8: 652685 | ERICH1 |
| cg00395995 | chr8: 674959 | ERICH1 |
| cg17176558 | chr8: 923949 | Not described |
| cg26246536 | chr8: 975053 | Not described |
| cg25308354 | chr8: 1048765 | Not described |
| cg26607620 | chr8: 1497078 | DLGAP2 |
| cg10296718 | chr8: 1848143 | ARHGEF10 |
| cg09126794 | chr8: 1879551 | ARHGEF10 |
| cg07503203 | chr8: 2005940 | MYOM2 |
| cg21847720 | chr8: 2075777 | MYOM2 |
| cg22764861 | chr8: 6277513 | MCPH1 |
| cg12174280 | chr8: 7543899 | Not described |
| cg06142662 | chr8: 9599057 | MIR597, TNKS |
| cg12567615 | chr8: 11609706 | GATA4 |
| cg24555670 | chr8: 18244502 | Not described |
| cg06944522 | chr8: 18663702 | PSD3 |
| cg13530105 | chr8: 19795537 | LPL |
| cg18593937 | chr8: 20161633 | Not described |
| cg03089940 | chr8: 21767146 | DOK2 |
| cg00599393 | chr8: 22457479 | C8orf58 |
| cg09473788 | chr8: 22833129 | Not described |
| cg08544307 | chr8: 28725934 | INTS9 |
| cg27128883 | chr8: 37438843 | Not described |
| cg12869334 | chr8: 37699360 | GPR124 |
| cg19140508 | chr8: 38259225 | LETM2 |
| cg26385256 | chr8: 38326334 | FGFR1 |
| cg07120174 | chr8: 41351213 | GOLGA7 |
| cg00564790 | chr8: 42206370 | POLB |
| cg26096333 | chr8: 42358481 | SLC20A2 |
| cg25033405 | chr8: 48610943 | KIAA0146 |
| cg22021882 | chr8: 52756855 | PCMTD1 |
| cg27399080 | chr8: 52809365 | PCMTD1 |
| cg27004342 | chr8: 56354098 | XKR4 |
| cg06983174 | chr8: 56433594 | XKR4 |
| cg13689756 | chr8: 59529329 | NSMAF |
| cg27272679 | chr8: 65294635 | Not described |
| cg04410024 | chr8: 75896798 | CRISPLD1 |
| cg04831599 | chr8: 78310503 | Not described |
| cg01257837 | chr8: 86874200 | Not described |
| cg26346210 | chr8: 98610507 | Not described |
| cg20662018 | chr8: 99126378 | HRSP12 |
| cg17103929 | chr8: 101225361 | SPAG1 |
| cg09393372 | chr8: 101966602 | YWHAZ |
| cg17534034 | chr8: 106586880 | ZFPM2 |
| cg19010198 | chr8: 107478131 | OXR1 |
| cg27616227 | chr8: 108510314 | ANGPT1 |
| cg06288869 | chr8: 112655155 | Not described |
| cg16074241 | chr8: 114079890 | CSMD3 |
| cg10066697 | chr8: 120724788 | Not described |
| cg25638515 | chr8: 122080710 | Not described |
| cg17213048 | chr8: 124332861 | ATAD2 |
| cg21849835 | chr8: 125333062 | TMEM65 |
| cg21390512 | chr8: 127568850 | FAM84B |
| cg02925049 | chr8: 127568854 | FAM84B |
| cg13636698 | chr8: 127569019 | FAM84B |
| cg16277922 | chr8: 131349729 | ASAP1 |
| cg12073251 | chr8: 141568652 | EIF2C2 |
| cg12039286 | chr8: 143533193 | Not described |
| cg17770910 | chr8: 143851427 | LYNX1 |
| cg00624939 | chr8: 144279703 | Not described |
| cg13529726 | chr8: 144416767 | TOP1MT |
| cg11519751 | chr8: 144438377 | TOP1MT |
| cg26857665 | chr8: 144543052 | ZC3H3 |
| cg01014113 | chr8: 144668641 | EEF1D |
| cg21206652 | chr8: 144790187 | LOC100130274 |
| cg01612292 | chr8: 144809598 | FAM83H |
| cg25468618 | chr8: 144810034 | FAM83H |
| cg01399317 | chr8: 144810339 | FAM83H |
| cg19497517 | chr8: 145028257 | PLEC1 |
| cg13517737 | chr8: 145831543 | KIAA1688 |
| cg04753829 | chr8: 145879308 | Not described |
| cg09320337 | chr8: 146033038 | ZNF517 |
| cg10350793 | chr9: 975600 | DMRT3 |
| cg23008518 | chr9: 6481971 | UHRF2 |
| cg13469590 | chr9: 19229767 | Not described |
| cg14355192 | chr9: 36258114 | GNE |
| cg14428530 | chr9: 72874528 | SMC5 |
| cg25355416 | chr9: 72926759 | SMC5 |
| cg17036899 | chr9: 73035608 | Not described |
| cg13863007 | chr9: 95766607 | FGD3 |
| cg14337668 | chr9: 99883682 | Not described |
| cg21182108 | chr9: 101553307 | ANKS6 |
| cg13576904 | chr9: 109768994 | ZNF462, MIR548Q |
| cg11233228 | chr9: 116037401 | PRPF4, CDC26 |
| cg14215970 | chr9: 124132919 | STOM |
| cg04739729 | chr9: 128510669 | PBX3 |
| cg13931285 | chr9: 129678298 | RALGPS1 |
| cg00801360 | chr9: 131450106 | SET |
| cg10026853 | chr9: 135031667 | Not described |
| cg14018414 | chr9: 136454238 | Not described |
| cg14209471 | chr9: 139317216 | PMPCA |
| cg21179618 | chr9: 139424642 | NOTCH1 |
| cg14224203 | chr9: 139716026 | C9orf86 |
| cg15206067 | chr9: 140312163 | EXD3 |
| cg18848151 | chr10: 5487582 | NET1 |
| cg09045979 | chr10: 5918682 | ANKRD16 |
| cg05456956 | chr10: 6557542 | PRKCQ |
| cg06220761 | chr10: 7455518 | Not described |
| cg12246472 | chr10: 7603859 | ITIH5 |
| cg07508934 | chr10: 11574571 | USP6NL |
| cg09926867 | chr10: 12680739 | CAMK1D |
| cg11542402 | chr10: 13141466 | OPTN |
| cg06809970 | chr10: 13523094 | BEND7 |
| cg03120475 | chr10: 15411913 | FAM171A1 |
| cg12427286 | chr10: 21653244 | Not described |
| cg12610471 | chr10: 22634199 | SPAG6 |
| cg11927951 | chr10: 22638314 | SPAG6 |
| cg11502597 | chr10: 24497713 | KIAA1217 |
| cg00348166 | chr10: 32769208 | CCDC7 |
| cg12273505 | chr10: 35534954 | CCNY |
| cg02780400 | chr10: 43187469 | Not described |
| cg14683378 | chr10: 43251951 | Not described |
| cg08821715 | chr10: 46991996 | Not described |
| cg25995980 | chr10: 46993515 | GPRIN2 |
| cg16775752 | chr10: 49674530 | ARHGAP22 |
| cg17712120 | chr10: 62150630 | ANK3 |
| cg13490244 | chr10: 67231801 | Not described |
| cg18612237 | chr10: 70514549 | SNORD98, CCAR1 |
| cg05456362 | chr10: 70937702 | Not described |
| cg13159413 | chr10: 75231932 | PPP3CB |
| cg10611310 | chr10: 75569127 | NDST2 |
| cg19917607 | chr10: 76288312 | ADK, LOC102723439 |
| cg25229706 | chr10: 76871606 | SAMD8 |
| cg06706894 | chr10: 80167002 | Not described |
| cg22715761 | chr10: 88428147 | LDB3 |
| cg15347156 | chr10: 88702795 | MMRN2 |
| cg27084903 | chr10: 89621920 | PTEN, KILLIN |
| cg04266774 | chr10: 90482997 | LIPK |
| cg03362798 | chr10: 95105687 | MYOF |
| cg10039734 | chr10: 95139986 | MYOF |
| cg01056004 | chr10: 98948259 | Not described |
| cg02792168 | chr10: 101281924 | Not described |
| cg26195366 | chr10: 102242535 | WNT8B |
| cg16448058 | chr10: 102880841 | TLX1NB |
| cg00793774 | chr10: 104403707 | TRIM8 |
| cg12700402 | chr10: 104470719 | ARL3 |
| cg15227982 | chr10: 104535854 | C10orf26 |
| cg26464592 | chr10: 105093625 | PCGF6 |
| cg11005552 | chr10: 105648138 | OBFC1 |
| cg00980194 | chr10: 111764646 | ADD3, ADD3AS1 |
| cg20114154 | chr10: 114438633 | VTI1A |
| cg25627693 | chr10: 120969332 | GRK5 |
| cg23748514 | chr10: 122262176 | PPAPDC1A |
| cg22762615 | chr10: 123358210 | FGFR2 |
| cg24571086 | chr10: 123371156 | Not described |
| cg08904986 | chr10: 123738373 | Not described |
| cg15428140 | chr10: 127220354 | Not described |
| cg12854248 | chr10: 127464544 | MMP21 |
| cg02938690 | chr10: 131576237 | Not described |
| cg06261400 | chr10: 131744476 | EBF3 |
| cg14683065 | chr10: 134149184 | LRRC27 |
| cg25268100 | chr10: 134457731 | INPP5A |
| cg27196695 | chr10: 134571377 | INPP5A |
| cg19623624 | chr10: 135278901 | LOC619207 |
| cg27354893 | chr11: 259533 | Not described |
| cg10261205 | chr11: 289927 | ATHL1 |
| cg04663285 | chr11: 379455 | B4GALNT4 |
| cg26608305 | chr11: 925416 | AP2A2 |
| cg04088212 | chr11: 2019859 | H19 |
| cg12077660 | chr11: 2721243 | KCNQ1OT1, KCNQ1 |
| cg03401726 | chr11: 2721248 | KCNQ1OT1, KCNQ1 |
| cg16739686 | chr11: 2721336 | KCNQ1OT1, KCNQ1 |
| cg08446215 | chr11: 2721366 | KCNQ1OT1, KCNQ1 |
| cg01873334 | chr11: 2721632 | KCNQ1OT1, KCNQ1 |
| cg26094482 | chr11: 2722073 | KCNQ1OT1, KCNQ1 |
| cg14945182 | chr11: 3012879 | NAP1L4 |
| cg04672275 | chr11: 3160193 | OSBPL5 |
| cg06041866 | chr11: 4566239 | OR52M1 |
| cg08935899 | chr11: 6651712 | DCHS1 |
| cg21328202 | chr11: 8194785 | Not described |
| cg08653973 | chr11: 8900217 | ST5 |
| cg18989133 | chr11: 17518482 | USH1C |
| cg10152271 | chr11: 17740986 | MYOD1 |
| cg06873166 | chr11: 18814391 | PTPN5 |
| cg26821579 | chr11: 22851416 | SVIP |
| cg08290675 | chr11: 23425026 | Not described |
| cg07067452 | chr11: 23425030 | Not described |
| cg03175849 | chr11: 31458270 | IMMP1L |
| cg09992216 | chr11: 32353565 | Not described |
| cg09234616 | chr11: 32452592 | WT1 |
| cg25933215 | chr11: 35208205 | CD44 |
| cg07675898 | chr11: 41681482 | Not described |
| cg23308107 | chr11: 47429939 | SLC39A13 |
| cg13942016 | chr11: 47447463 | PSMC3 |
| cg18356785 | chr11: 47611780 | C1QTNF4 |
| cg14784876 | chr11: 49456201 | Not described |
| cg05465448 | chr11: 55586744 | OR5D18 |
| cg21878015 | chr11: 56059314 | OR8H1 |
| cg11104828 | chr11: 58869979 | Not described |
| cg03298096 | chr11: 59596955 | GIF |
| cg09630109 | chr11: 61914262 | INCENP |
| cg16539346 | chr11: 64571673 | MEN1, MAP4K2 |
| cg16749093 | chr11: 65352605 | EHBP1L1 |
| cg01180479 | chr11: 65374833 | MAP3K11 |
| cg25294185 | chr11: 65487814 | RNASEH2C |
| cg15995296 | chr11: 67210812 | CORO1B |
| cg12652780 | chr11: 70178166 | PPFIA1 |
| cg06308369 | chr11: 71263389 | Not described |
| cg08909363 | chr11: 71892539 | Not described |
| cg24878173 | chr11: 72533202 | ATG16L2 |
| cg13771313 | chr11: 72533295 | ATG16L2 |
| cg03717994 | chr11: 73114164 | Not described |
| cg15003737 | chr11: 77182075 | PAK1 |
| cg26893231 | chr11: 89322707 | NOX4 |
| cg25299011 | chr11: 89463537 | Not described |
| cg14926625 | chr11: 92084998 | FAT3 |
| cg06907989 | chr11: 92971815 | Not described |
| cg05130679 | chr11: 94502824 | AMOTL1 |
| cg17739345 | chr11: 102927599 | DCUN1D5 |
| cg10855540 | chr11: 110056594 | Not described |
| cg04009816 | chr11: 111397900 | C11orf88 |
| cg23577102 | chr11: 111411348 | LAYN |
| cg01586609 | chr11: 113846937 | HTR3A |
| cg20997773 | chr11: 117667841 | DSCAML1 |
| cg20341535 | chr11: 118017101 | SCN4B |
| cg11361260 | chr11: 118901601 | SLC37A4 |
| cg15056794 | chr11: 121987155 | LOC399959, BLID |
| cg11596580 | chr12: 3600764 | PRMT8 |
| cg12495801 | chr12: 6560851 | TAPBPL, CD27, LOC678655 |
| cg25136495 | chr12: 6729718 | LPAR5 |
| cg19360943 | chr12: 6762431 | ING4 |
| cg17474934 | chr12: 8938572 | Not described |
| cg24486842 | chr12: 14997216 | ART4 |
| cg10783206 | chr12: 19636888 | AEBP2 |
| cg21330944 | chr12: 27149914 | TM7SF3 |
| cg07216656 | chr12: 29506350 | ERGIC2 |
| cg01413382 | chr12: 32714874 | FGD4 |
| cg15056189 | chr12: 49176428 | ADCY6 |
| cg01656996 | chr12: 49526897 | Not described |
| cg03460053 | chr12: 51926890 | Not described |
| cg06573124 | chr12: 53689274 | PFDN5 |
| cg03668274 | chr12: 54426903 | HOXC4, HOXC5 |
| cg12502577 | chr12: 57615581 | NXPH4 |
| cg19542346 | chr12: 58145283 | CDK4 |
| cg19185907 | chr12: 60660457 | Not described |
| cg01643123 | chr12: 63207179 | PPM1H |
| cg14078059 | chr12: 65174660 | Not described |
| cg23390381 | chr12: 65488095 | WIF1 |
| cg16560376 | chr12: 70132281 | RAB3IP, LOC101928002 |
| cg24713959 | chr12: 71709550 | Not described |
| cg09972192 | chr12: 72667326 | LOC283392, TRHDE |
| cg26056477 | chr12: 76963168 | Not described |
| cg21484956 | chr12: 77273469 | CSRP2 |
| cg24981283 | chr12: 79188000 | Not described |
| cg01588748 | chr12: 79258386 | SYT1 |
| cg24918622 | chr12: 88498981 | CEP290 |
| cg15282731 | chr12: 90177012 | Not described |
| cg15712226 | chr12: 96252066 | SNRPF |
| cg23429240 | chr12: 109040983 | CORO1C |
| cg13466660 | chr12: 110435302 | GIT2 |
| cg11811816 | chr12: 111713664 | CUX2 |
| cg11246938 | chr12: 115112433 | TBX3 |
| cg15235945 | chr12: 118884161 | Not described |
| cg24675568 | chr12: 120018195 | Not described |
| cg18724891 | chr12: 121147746 | UNC119B |
| cg02516442 | chr12: 122714821 | Not described |
| cg03216691 | chr12: 123466396 | ARL6IP4 |
| cg10236435 | chr12: 123944014 | SNRNP35 |
| cg10609442 | chr12: 125010075 | NCOR2 |
| cg06741568 | chr12: 129028641 | TMEM132C |
| cg19956540 | chr12: 130399420 | Not described |
| cg15612205 | chr12: 130516192 | Not described |
| cg04227871 | chr12: 131002792 | RIMBP2 |
| cg26098972 | chr12: 131166906 | Not described |
| cg14525755 | chr12: 132671672 | Not described |
| cg14695497 | chr12: 132860966 | GALNT9 |
| cg09698465 | chr12: 133000178 | Not described |
| cg08991210 | chr12: 133010785 | Not described |
| cg06553975 | chr12: 133085262 | FBRSL1 |
| cg07148716 | chr13: 20806539 | GJB6 |
| cg16896687 | chr13: 20966332 | Not described |
| cg14395298 | chr13: 23412250 | Not described |
| cg25366582 | chr13: 25621027 | Not described |
| cg26941801 | chr13: 32882845 | ZAR1L |
| cg00566450 | chr13: 34839416 | Not described |
| cg09911332 | chr13: 44542478 | Not described |
| cg06538223 | chr13: 48513661 | Not described |
| cg26766132 | chr13: 51860984 | Not described |
| cg27581373 | chr13: 51996118 | INTS6 |
| cg06158646 | chr13: 52248304 | WDFY2 |
| cg02342250 | chr13: 73438452 | PIBF1 |
| cg12034573 | chr13: 73939091 | Not described |
| cg03602567 | chr13: 97642682 | OXGR1 |
| cg09283763 | chr13: 99740027 | DOCK9, DOCK9AS2 |
| cg22203300 | chr13: 100092028 | Not described |
| cg18870995 | chr13: 103054146 | FGF14 |
| cg13406762 | chr13: 109144360 | Not described |
| cg25137436 | chr13: 113299913 | C13orf35 |
| cg09294084 | chr13: 113646732 | MCF2L |
| cg06354455 | chr13: 114054873 | Not described |
| cg20017856 | chr14: 29990921 | MIR548AI |
| cg04682600 | chr14: 31874845 | HEATR5A |
| cg01074354 | chr14: 33244107 | AKAP6 |
| cg11382630 | chr14: 35747081 | PSMA6 |
| cg00329695 | chr14: 37643265 | SLC25A21 |
| cg14353218 | chr14: 48095792 | MDGA2 |
| cg14200649 | chr14: 50121240 | POLE2 |
| cg18095720 | chr14: 50160168 | KLHDC1 |
| cg18000586 | chr14: 51067155 | ATL1 |
| cg11836171 | chr14: 51448790 | TRIM9 |
| cg10257302 | chr14: 55907427 | TBPL2 |
| cg26140264 | chr14: 65039015 | PPP1R36 |
| cg01772385 | chr14: 65347518 | Not described |
| cg20250396 | chr14: 65880036 | FUT8, LOC645431 |
| cg19937979 | chr14: 70039915 | Not described |
| cg05028768 | chr14: 75469467 | EIF2B2 |
| cg18628686 | chr14: 83507399 | Not described |
| cg12504912 | chr14: 90081872 | FOXN3 |
| cg13225565 | chr14: 93106414 | RIN3 |
| cg15443535 | chr14: 93154317 | RIN3 |
| cg13080172 | chr14: 99641778 | BCL11B |
| cg04676846 | chr14: 100196947 | Not described |
| cg09860529 | chr14: 100933062 | WDR25 |
| cg09918310 | chr14: 102275227 | PPP2R5C |
| cg14531093 | chr14: 102973717 | ANKRD9 |
| cg18587137 | chr14: 103593503 | TNFAIP2 |
| cg10501093 | chr14: 103593520 | TNFAIP2 |
| cg06154002 | chr14: 104394776 | TDRD9 |
| cg06289566 | chr14: 104394782 | TDRD9 |
| cg04904561 | chr14: 104394831 | TDRD9 |
| cg05210671 | chr14: 104787388 | Not described |
| cg16744228 | chr15: 25200113 | SNRPN, SNURF |
| cg04072648 | chr15: 25200145 | SNURF, SNRPN |
| cg17503663 | chr15: 25200202 | SNURF, SNRPN |
| cg11686940 | chr15: 31664433 | KLF13 |
| cg01615424 | chr15: 32639059 | Not described |
| cg13172554 | chr15: 35917803 | Not described |
| cg10223549 | chr15: 36995676 | C15orf41 |
| cg06700122 | chr15: 40214716 | Not described |
| cg17460228 | chr15: 41052250 | Not described |
| cg13481974 | chr15: 42174516 | SPTBN5 |
| cg17395184 | chr15: 42750462 | ZFP106 |
| cg02042600 | chr15: 42800833 | SNAP23 |
| cg09296044 | chr15: 43477606 | CCNDBP1 |
| cg24530489 | chr15: 56299380 | Not described |
| cg05907649 | chr15: 56519080 | RFX7 |
| cg25688583 | chr15: 57510460 | TCF12 |
| cg04501263 | chr15: 59216658 | SLTM |
| cg20170632 | chr15: 62320024 | VPS13C |
| cg04438332 | chr15: 63889271 | FBXL22, USP3AS1 |
| cg06674527 | chr15: 65369928 | LOC390594 |
| cg13997780 | chr15: 66816387 | ZWILCH |
| cg24032190 | chr15: 67442893 | SMAD3 |
| cg20673820 | chr15: 70935237 | Not described |
| cg18121641 | chr15: 70991223 | UACA |
| cg25356006 | chr15: 72472792 | GRAMD2 |
| cg24648408 | chr15: 72690143 | TMEM202 |
| cg05622550 | chr15: 74315474 | PML |
| cg25281920 | chr15: 76586337 | ETFA |
| cg21322248 | chr15: 77289047 | PSTPIP1 |
| cg26798213 | chr15: 78786261 | IREB2 |
| cg22753661 | chr15: 79092743 | ADAMTS7 |
| cg12546081 | chr15: 79602873 | TMED3 |
| cg02326224 | chr15: 81474451 | Not described |
| cg12651540 | chr15: 90191926 | KIF7 |
| cg11992265 | chr15: 90818329 | Not described |
| cg04795110 | chr15: 90978852 | IQGAP1 |
| cg12630461 | chr15: 91500009 | RCCD1 |
| cg18739950 | chr15: 95870440 | Not described |
| cg00105080 | chr15: 99277733 | IGF1R |
| cg07199183 | chr15: 100879199 | ADAMTS17 |
| cg27548659 | chr15: 101069624 | CERS3 |
| cg03459668 | chr16: 545622 | RAB11FIP3 |
| cg09323788 | chr16: 843219 | CHTF18 |
| cg14158665 | chr16: 1968276 | HS3ST6 |
| cg11898029 | chr16: 2097888 | TSC2, NTHL1 |
| cg07102406 | chr16: 2294639 | DCI |
| cg10492999 | chr16: 3063894 | CLDN9 |
| cg15821035 | chr16: 3333461 | ZNF263 |
| cg27552378 | chr16: 3702776 | DNASE1 |
| cg08336138 | chr16: 4934475 | PPL |
| cg01369829 | chr16: 22029878 | C16orf52 |
| cg08735200 | chr16: 28194832 | XPO6 |
| cg00446595 | chr16: 28331871 | SBK1 |
| cg08697180 | chr16: 30409538 | ZNF48 |
| cg08351781 | chr16: 34969983 | Not described |
| cg08290471 | chr16: 51788337 | Not described |
| cg05004142 | chr16: 57562740 | CCDC102A |
| cg02102533 | chr16: 57562841 | CCDC102A |
| cg05948940 | chr16: 68481342 | SMPD3 |
| cg00505936 | chr16: 69139418 | HAS3 |
| cg00683085 | chr16: 69213433 | Not described |
| cg03865041 | chr16: 71842666 | AP1G1 |
| cg07786668 | chr16: 73092391 | ZFHX3 |
| cg09398138 | chr16: 75060108 | ZNRF1 |
| ch.16.74794652R | chr16: 76237151 | Not described |
| cg22357511 | chr16: 76702560 | Not described |
| cg03662545 | chr16: 85254209 | Not described |
| cg07598272 | chr16: 86597244 | Not described |
| cg26934960 | chr16: 87228921 | Not described |
| cg22025478 | chr16: 87525515 | ZCCHC14 |
| cg08196561 | chr16: 87525539 | ZCCHC14 |
| cg12436851 | chr16: 87525542 | ZCCHC14 |
| cg27182321 | chr16: 88163807 | Not described |
| cg02656871 | chr16: 88442535 | Not described |
| cg07397579 | chr16: 88459662 | Not described |
| cg03286774 | chr16: 89590357 | SPG7 |
| cg17277337 | chr17: 181997 | LOC100506388, RPH3AL |
| cg12197459 | chr17: 686450 | GLOD4, RNMTL1 |
| cg14748193 | chr17: 1035308 | ABR |
| cg02482001 | chr17: 6338476 | AIPL1 |
| cg17658923 | chr17: 7145735 | GABARAP |
| cg10092265 | chr17: 7311742 | NLGN2 |
| cg09568217 | chr17: 7311868 | NLGN2 |
| cg01412970 | chr17: 17109239 | PLD6 |
| cg06775073 | chr17: 17603749 | RAM1 |
| cg02147681 | chr17: 17603837 | RAI1 |
| cg23584010 | chr17: 18792479 | PRPSAP2 |
| cg04498198 | chr17: 27899966 | TP53113 |
| cg12193833 | chr17: 30244370 | Not described |
| cg04300553 | chr17: 33734847 | Not described |
| cg22534110 | chr17: 37764706 | NEUROD2 |
| cg11693508 | chr17: 37793320 | STARD3 |
| cg05842113 | chr17: 38075648 | GSDMB |
| cg07856823 | chr17: 38979675 | KRT10, TMEM99 |
| cg16426293 | chr17: 40192112 | Not described |
| cg15909132 | chr17: 42431109 | FAM171A2 |
| cg19935040 | chr17: 42432165 | FAM171A2 |
| cg14271505 | chr17: 42767560 | CCDC43 |
| cg08452456 | chr17: 45786439 | TBKBP1 |
| cg13479204 | chr17: 46641708 | HOXB3 |
| cg15718595 | chr17: 47693731 | SPOP |
| cg26890094 | chr17: 48464305 | LRRC59 |
| cg20633883 | chr17: 48712051 | ABCC3 |
| cg23719290 | chr17: 56912132 | PPM1E |
| cg11619775 | chr17: 61920126 | SMARCD2 |
| cg12097883 | chr17: 62774939 | LOC146880 |
| cg09388215 | chr17: 64188148 | CCDC46 |
| cg05732683 | chr17: 67058395 | ABCA9 |
| cg14254769 | chr17: 72744909 | SLC9A3R1 |
| cg23834812 | chr17: 76500801 | DNAH17 |
| cg01385356 | chr17: 76837375 | USP36 |
| cg26124560 | chr17: 78064152 | CCDC40 |
| cg16980380 | chr17: 78417883 | Not described |
| cg01623261 | chr17: 79377850 | BAHCC1 |
| cg23161691 | chr17: 79428986 | BAHCC1 |
| cg22175624 | chr17: 80829261 | TBCD |
| cg22750845 | chr17: 80966690 | B3GNTL1 |
| cg26958509 | chr18: 12478776 | SPIRE1 |
| cg03400437 | chr18: 22006455 | IMPACT |
| cg12606400 | chr18: 29172689 | TTR |
| cg13428397 | chr18: 32672280 | MAPRE2 |
| cg05212403 | chr18: 53068637 | TCF4 |
| cg21911276 | chr18: 55289797 | NARS |
| cg23182655 | chr18: 60301872 | Not described |
| cg11416737 | chr18: 60877850 | BCL2 |
| cg25016143 | chr18: 64736132 | Not described |
| cg24570779 | chr18: 70999178 | LOC100505817 |
| cg24532901 | chr18: 77782597 | TXNL4A |
| cg06595479 | chr19: 519035 | C19orf20 |
| cg08269974 | chr19: 853054 | ELANE |
| cg05620923 | chr19: 1466647 | APC2 |
| cg11809123 | chr19: 1584870 | MBD3 |
| cg01980928 | chr19: 1952821 | C19orf34, CSNK1G2 |
| cg25654695 | chr19: 2273216 | OAZ1 |
| cg21869609 | chr19: 2291613 | LINGO3 |
| cg26843498 | chr19: 2361574 | Not described |
| cg10350536 | chr19: 2650863 | GNG7 |
| cg03224385 | chr19: 3250946 | CELF5 |
| cg21206277 | chr19: 4048075 | ZBTB7A |
| cg24699296 | chr19: 7747220 | TRAPPC5 |
| cg04487907 | chr19: 9326161 | OR7D4 |
| cg19740458 | chr19: 12848676 | ASNA1 |
| cg09638793 | chr19: 13261286 | STX10, IER2 |
| cg08846870 | chr19: 15568360 | RASAL3 |
| cg26669806 | chr19: 18899483 | COMP |
| cg21725265 | chr19: 19051201 | HOMER3 |
| cg02682777 | chr19: 19336413 | NCAN |
| cg26732615 | chr19: 19648335 | CILP2, YJEFN3 |
| cg20246743 | chr19: 21752023 | Not described |
| cg05965106 | chr19: 31828728 | TSHZ3 |
| cg17355466 | chr19: 35596858 | HPNAS1 |
| cg09178470 | chr19: 35596864 | HPNAS1 |
| cg05778847 | chr19: 38746538 | PPP1R14A |
| cg00002033 | chr19: 39798481 | LRFN1 |
| cg13982417 | chr19: 40854131 | PLD3, C19orf47 |
| cg10775753 | chr19: 42081388 | CEACAM21 |
| cg24267358 | chr19: 42299379 | CEACAM3 |
| cg00449767 | chr19: 45737603 | EXOC3L2 |
| cg01565314 | chr19: 45737610 | EXOC3L2 |
| cg09450024 | chr19: 45737623 | EXOC3L2 |
| cg08882547 | chr19: 45737792 | EXOC3L2 |
| cg14876077 | chr19: 45737873 | EXOC3L2 |
| cg12009516 | chr19: 45737880 | EXOC3L2 |
| cg07769957 | chr19: 46443657 | NOVA2 |
| cg23851638 | chr19: 47803550 | Not described |
| cg03950599 | chr19: 48698419 | Not described |
| cg15985418 | chr19: 49173732 | SEC1, NTN5 |
| cg08056423 | chr19: 50155043 | SCAF1 |
| cg27500647 | chr19: 51602230 | CTU1 |
| cg03643149 | chr19: 54041519 | ZNF331 |
| cg13922166 | chr19: 54693569 | MBOAT7, TSEN34 |
| cg23226510 | chr19: 56042469 | SBK2 |
| cg15777825 | chr19: 57352014 | ZIM2, PEG3, MIMT1 |
| cg12205903 | chr19: 57352021 | ZIM2, PEG3, MIMT1 |
| cg13598480 | chr20: 3145356 | ProSAPiP1 |
| cg20821980 | chr20: 3145359 | ProSAPiP1 |
| cg22534145 | chr20: 23015936 | SSTR4 |
| cg07722523 | chr20: 25071554 | Not described |
| cg11129060 | chr20: 32205882 | CBFA2T2 |
| cg11092487 | chr20: 33585188 | MYH7B |
| cg11469779 | chr20: 43992721 | SYS1, SYS1DBNDD2 |
| cg27594511 | chr20: 52270424 | Not described |
| cg27262236 | chr20: 55965077 | RBM38 |
| cg12194708 | chr20: 57406069 | GNASAS1 |
| cg26711395 | chr20: 57430133 | GNAS |
| cg17652507 | chr20: 57463653 | GNAS |
| cg22407822 | chr20: 57463658 | GNAS |
| cg15222215 | chr20: 57463783 | GNAS |
| cg14263118 | chr20: 57463787 | GNAS |
| cg11244758 | chr20: 57463900 | GNAS |
| cg05926269 | chr20: 57463906 | GNAS |
| cg03821543 | chr20: 57463925 | GNAS |
| cg23159236 | chr20: 57464002 | GNAS |
| cg22639787 | chr20: 57464973 | GNAS |
| cg26493612 | chr20: 58508390 | FAM217B, SYCP2 |
| cg15961533 | chr21: 17039383 | Not described |
| cg18455081 | chr21: 17144413 | USP25 |
| cg26153954 | chr21: 27543721 | APP |
| cg08876401 | chr21: 34865077 | DNAJC28 |
| cg26890676 | chr21: 34945428 | SON |
| cg15709587 | chr21: 35896737 | RCAN1 |
| cg01664727 | chr21: 36258423 | RUNX1 |
| cg00994804 | chr21: 36259383 | RUNX1 |
| cg03040477 | chr21: 38753088 | DYRK1A |
| cg06464686 | chr21: 47371503 | Not described |
| cg13126279 | chr21: 47581558 | C21orf56 |
| cg02357751 | chr22: 19710880 | GP1BB, SEPT5 |
| cg24216792 | chr22: 22123753 | MAPK1 |
| cg08732993 | chr22: 24797222 | SPECC1LADORA2A, |
| | | SPECC1L |
| cg13013297 | chr22: 29471100 | KREMEN1 |
| cg03452581 | chr22: 30307656 | MTMR3 |
| cg12253469 | chr22: 37420454 | MPST |
| cg26038465 | chr22: 38610514 | MAFF |
| cg15548613 | chr22: 38610795 | MAFF |
| cg01029450 | chr22: 43253559 | ARFGAP3 |
| cg11769406 | chr22: 43547298 | TSPO |
| cg17024708 | chr22: 44258638 | SULT4A1 |
| cg18036763 | chr22: 45404910 | PHF21B |
| cg04566512 | chr22: 46457588 | Not described |
| cg01271126 | chr22: 48718268 | Not described |
| cg13192640 | chr22: 49096685 | FAM19A5 |
| cg16981421 | chr22: 50529071 | MOV10L1 |
| cg06357843 | chr22: 50616150 | PANX2 |
| cg17547708 | chr22: 51021506 | LOC100144603, |
| | | CHKBCPT1B, CHKB |
| cg13213810 | chr22: 51158720 | SHANK3 |
| cg06422471 | chr22: 51169500 | SHANK3 |

### CITATION LIST

### Patent Literature

WO2020092455 (The Broad Inst. Inc. et al.)
WO2018209324 ((The Broad Inst. Inc. et al.)
WO2020170231 (St. Jude's Children Research Institute)

### Non Patent Literature

Majzner R G, Mackall C L., “Tumor antigen escape from CAR T-cell therapy”, Cancer Discov 2018; 8: 1219-26.
Fraietta et al., “Determinants of response and resistance to CD19 chimeric antigen receptor (CAR) T cell therapy of chronic lymphocytic leukemia”, Nat Med 2018; 24: 563-71.
Fraietta et al., “Disruption of TET2 promotes the therapeutic efficacy of CD19-targeted T cells”, Nature 2018; 558: 307-12; or by Nobles et al., in “CD19-targeting CAR T cell immunotherapy outcomes correlate with genomic modification by vector integration” J Clin Invest 2020; 130: 673-85.
Rossi et al., “Preinfusion polyfunctional anti-CD19 chimeric antigen receptor T cells are associated with clinical outcomes in NHL”, Blood 2018; 132: 804-14.
Jacoby et al. Locally produced CD19 CAR T-cells leading to clinical remissions in medullary and extramedullary relapsed acute lymphoblastic leukemia. Am J Hematol; 93: 1485-92;
Itzhaki et al., Head-to-head comparison of in-house produced CD19 CAR-T cell in ALL and NHL patients. J Immunother Cancer 2020; 8: e000148
Moran et al., Validation of a DNA methylation microarray for 850,000 CpG sites of the human genome enriched in enhancer sequences. Epigenomics 2016; 8: 389-99
Deng et al., Characteristics of anti-CD19 CART cell infusion products associated with efficacy and toxicity in patients with large B cell lymphomas. Nat Med 2020; published online October 5. https://doi.org/10.1038/s41591-020-1061-7.
You et al., Decitabine-mediated epigenetic reprograming enhances anti-leukemia efficacy of CD123-targeted chimeric antigen receptor T-cells. Front Immunol 2020; 11: 1787.
Ortíz-Maldonado V, Rives S, Castellà M, et al. CART19-BE-01: A Multicenter Trial of ARI-0001 Cell Therapy in Patients with CD19+ Relapsed/Refractory Malignancies. Mol Ther. 2021; 29(2):636-644.
Quintarelli C, Guercio M, Manni S, et al. Strategy to prevent epitope masking in CAR.CD19+B-cell leukemia blasts. J. Immunother. Cancer. 2021; 9(6):e001514.
Lee D W, Santomasso B D, Locke F L, et al. ASTCT Consensus Grading for Cytokine Release Syndrome and Neurologic Toxicity Associated with Immune Effector Cells. Biol Blood Marrow Transplant. 2019; 25(4):625-638.
Weber E W, Parker K R, Sotillo E, et al. Transient rest restores functionality in exhausted CAR-T cells through epigenetic remodeling. Science. 2021; 372(6537):eaba1786.
Duruisseaux M, Martinez-Cardús A, Calleja-Cervantes M E, et al. Epigenetic prediction of response to anti-PD-1 treatment in non-small-cell lung cancer: a multicentre, retrospective analysis. Lancet Respir Med. 2018; 6(10):771-781.

What is claimed is:

1. An in vitro method for predicting the response of a subject to autologous chimeric antigen receptor T-cell (CAR T-cell) therapy, the method comprising:
(a) determining the methylation status of one or more cytosines in CpG sites of CAR T-cells, said CAR T-cells obtained from an isolated sample of the subject comprising T cells that, once isolated, have been transduced with the CAR, the one or more cytosines in CpG sites of CAR T-cells selected from the group consisting of: cytosine at position 86332162 of human chromosome 2, cytosine at position 188676237 of human chromosome 1; cytosine at position 105907265 of human chromosome 6; cytosine at position 234087867 of human chromosome 1; cytosine at position 32353565 of human chromosome 11; cytosine at position 22634199 of human chromosome 10: cytosine at position 45028225 of human chromosome 2; cytosine at position 220414164 of human chromosome 1; cytosine at position 209809 of human chromosome 6; cytosine at position 62905816 of human chromosome 1; cytosine at position 79780164 of human chromosome 6; and cytosine at position 28725934 of human chromosome 8, all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of response to CAR T-cell therapy and determining the subject will respond to CAR T-cell therapy if the methylation status is of the one or more CpG sites equal to said reference value or within the range of reference values.

2. The in vitro method according to claim 1, wherein the methylation status of the following CpG sites of CAR T cells is determined: cytosine at position 86332162 of human chromosome 2, cytosine at position 188676237 of human chromosome 1; cytosine at position 105907265 of human chromosome 6; cytosine at position 234087867 of human chromosome 1; cytosine at position 32353565 of human chromosome 11; and cytosine at position 22634199 of human chromosome 10.

3. The in vitro method according to claim 1, further comprising determining the methylation status of one or more CpG sites of CAR T-cells, said CAR T-cells obtained from an isolated sample of the subject comprising T-cells that, once isolated, have been transduced with the CAR, the one or more CpG sites of CAR T-cells selected from the group consisting of: cytosine at position 95139986 of human chromosome 10; cytosine at position 127612751 of human chromosome 6; cytosine at position 131058184 of human chromosome 2; cytosine at position 90081872 of human chromosome 14; cytosine at position 123944014 of human chromosome 12; cytosine at position 134457731 of human chromosome 10; cytosine at position 46993515 of human chromosome 10; cytosine at position 209809 of human chromosome 6; cytosine at position 122144477 of human chromosome 2; cytosine at position 6643814 of human chromosome 6; cytosine at position 60877850 of human chromosome 18; and cytosine at position 42299379 of human chromosome 19. all cytosine positions in human chromosomes according to the chromosome map and sequence entries of database UCSC Genome Browser on Human February 2009, GRCh37/hg19 assembly of the University of California Santa Cruz (UCSC).

4. The in vitro method according to claim 1, wherein the methylation status of the following 18 CpG sites of CAR T cells is determined: cytosine at position 86332162 of human chromosome 2, cytosine at position 188676237 of human chromosome 1; cytosine at position 105907265 of human chromosome 6; cytosine at position 234087867 of human chromosome 1; cytosine at position 32353565 of human chromosome 11; cytosine at position 22634199 of human chromosome 10; cytosine at position 95139986 of human chromosome 10; cytosine at position 127612751 of human chromosome 6; cytosine at position 131058184 of human chromosome 2; cytosine at position 90081872 of human chromosome 14; cytosine at position 123944014 of human chromosome 12; cytosine at position 134457731 of human chromosome 10; cytosine at position 46993515 of human chromosome 10; cytosine at position 209809 of human chromosome 6; cytosine at position 122144477 of human chromosome 2; cytosine at position 6643814 of human chromosome 6; cytosine at position 60877850 of human chromosome 18; and cytosine at position 42299379 of human chromosome 19.

5. The in vitro method according to claim 1, the method comprising:
(a) determining the methylation status of one or more cytosines in CpG sites of CAR T-cells, said CAR T-cells obtained from an isolated sample of the subject comprising T cells that, once isolated, have been transduced with the CAR, the one or more cytosines in CpG sites of CAR T-cells selected from the group consisting of: cytosine at position 86332162 of human chromosome 2, cytosine at position 188676237 of human chromosome 1; cytosine at position 45028225 of human chromosome 2; cytosine at position 220414164 of human chromosome 1; cytosine at position 209809 of human chromosome 6; cytosine at position 62905816 of human chromosome 1; cytosine at position 79780164 of human chromosome 6; and cytosine at position 28725934 of human chromosome 8, all cytosine positions in human chromosomes according to the chromosome map and sequence entries of database UCSC Genome Browser on Human February 2009, GRCh37/hg19 assembly of the University of California Santa Cruz (UCSC); and
(b) comparing the methylation status of the one or more CpG sites with a reference value or range of values of response to CAR T-cell therapy, and determining the subject will respond to CAR T-cell therapy if the methylation status is of the one or more CpG sites equal to said reference value or within the range of reference values.

6. The in vitro method according to claim 5, wherein the methylation status of the following CpG sites of CAR T cells is determined: cytosine at position 86332162 of human chromosome 2, cytosine at position 188676237 of human chromosome 1; cytosine at position 45028225 of human chromosome 2; cytosine at position 220414164 of human chromosome 1; cytosine at position 209809 of human chromosome 6; cytosine at position 62905816 of human chromosome 1; and cytosine at position 79780164 of human chromosome 6.

7. The in vitro method according to claim 5, further comprising determining the methylation status of one or more CpG sites of CAR T-cells, said CAR T-cells obtained from an isolated sample of the subject comprising T-cells that, once isolated, have been transduced with the CAR, the one or more CpG sites of CAR T-cells selected from the group consisting of: cytosine at position 134457731 of human chromosome 10; cytosine at position 127612751 of human chromosome 6, cytosine at position 6643814 of human chromosome 6; cytosine at position 42299379 of human chromosome 19; cytosine at position 32353565 of human chromosome 11; cytosine at position 234087867 of human chromosome 1; cytosine at position 105907265 of human chromosome 6; cytosine at position 22634199 of human chromosome 10; cytosine at position 95870440 of human chromosome 15; cytosine at position 104470719 of human chromosome 10; cytosine at position 43253559 of human chromosome 22; cytosine at position 122144477 of human chromosome 2; cytosine at position 131166906 of human chromosome 12; cytosine at position 68481342 of human chromosome 16; cytosine at position 100879199 of human chromosome 15; cytosine at position 95139986 of human chromosome 10; cytosine at position 183063459 of human chromosome 4; cytosine at position 180614858 of human chromosome 5; cytosine at position 134571377 of human chromosome 10; cytosine at position 3600764 of human chromosome 12; cytosine at position 90081872 of human chromosome 14; cytosine at position 133000178 of human chromosome 12; cytosine at position 46993515 of human chromosome 10; cytosine at position 19229767 of human chromosome 9; and cytosine at position 24229300 of human chromosome 1, all cytosine positions in human chromosomes according to the chromosome map and sequence entries of database UCSC Genome Browser on Human February 2009, GRCh37/hg19 assembly of the University of California Santa Cruz (UCSC).

8. The in vitro method according to claim 5, wherein the methylation status of the following 32 CpG sites of CAR T cells is determined: cytosine at position 86332162 of human chromosome 2, cytosine at position 188676237 of human chromosome 1; cytosine at position 45028225 of human chromosome 2; cytosine at position 220414164 of human chromosome 1; cytosine at position 209809 of human chromosome 6; cytosine at position 62905816 of human chromosome 1; cytosine at position 79780164 of human chromosome 6; cytosine at position 134457731 of human chromosome 10; cytosine at position 127612751 of human chromosome 6, cytosine at position 6643814 of human chromosome 6; cytosine at position 42299379 of human chromosome 19; cytosine at position 32353565 of human chromosome 11; cytosine at position 234087867 of human chromosome 1; cytosine at position 105907265 of human chromosome 6; cytosine at position 22634199 of human chromosome 10; cytosine at position 95870440 of human chromosome 15; cytosine at position 104470719 of human chromosome 10; cytosine at position 43253559 of human chromosome 22; cytosine at position 122144477 of human chromosome 2; cytosine at position 131166906 of human chromosome 12; cytosine at position 68481342 of human chromosome 16; cytosine at position 100879199 of human chromosome 15; cytosine at position 95139986 of human chromosome 10; cytosine at position 183063459 of human chromosome 4; cytosine at position 180614858 of human chromosome 5; cytosine at position 134571377 of human chromosome 10; cytosine at position 3600764 of human chromosome 12; cytosine at position 90081872 of human chromosome 14; cytosine at position 133000178 of human chromosome 12; cytosine at position 46993515 of human chromosome 10; cytosine at position 19229767 of human chromosome 9; and cytosine at position 24229300 of human chromosome 1.

9. The in vitro method according to claim 1, wherein the isolated sample is selected from a biofluid including lymphocyte T cells.

10. The in vitro method according to claim 1, wherein the CAR is one targeting the B-lymphocyte antigen CD19 (UNIPROT P15391).

11. The in vitro method according to claim 1, wherein methylation status of the one or more cytosines in the CpG sites is determined with a set of DNA oligonucleotides comprising one or more oligonucleotides that are complementary to a sequence comprising the cytosine of each CpG and producing a differential signal if the cytosine in a determined position is methylated or unmethylated; or
wherein methylation status of the one or more cytosines in the CpG sites is determined with a set of DNA oligonucleotides comprising one or more oligonucleotides that are complementary to a sequence comprising the methylated cytosine of each CpG site, and one or more oligonucleotides that are complementary to a sequence comprising the unmethylated cytosine of each CpG site.

12. The in vitro method according to claim 1, wherein the response of a subject to chimeric antigen receptor T cell (CAR T cell) therapy response is a complete response, which means that no remission of the disease is observed after therapy.

13. The in vitro method according to claim 1, wherein the subject is suffering from B-cell malignancies.

14. The in vitro method according to claim 1, further comprising determining the methylation status of one or more CpG sites of the CAR T-cells obtained by transduction of the T cells in the isolated sample, the one or more CpG sites of CAR T cells selected from the group consisting of: cytosine at position 131058184 of human chromosome 2; cytosine at position 36259383 of human chromosome 21; cytosine at position 180614858 of human chromosome 5; cytosine at position 18899483 of human chromosome 19; cytosine at position 190448126 of human chromosome 1; cytosine al position 201123894 of human chromosome 1; cytosine at position 45505849 of human chromosome 3; cytosine at position 2075777 of human chromosome 8; cytosine at position 218340518 of human chromosome 2; cytosine at position 85637673 of human chromosome 2; cytosine at position 10415636 of human chromosome 6; cytosine at position 29990921 of human chromosome 14; cytosine at position 100879199 of human chromosome 15; cytosine at position 105648138 of human chromosome 10; cytosine at position 637813 of human chromosome 8; cytosine at position 110721138 of human chromosome 6; and cytosine at position 130516192 of human chromosome 12, all cytosine positions in human chromosomes according to the chromosome map and sequence entries of database UCSC Genome Browser on Human February 2009, GRCh37/hg19 assembly of the University of California Santa Cruz (UCSC).

15. The in vitro method according to claim 14, wherein the one or more CpG sites of CAR T cells selected from the group consisting of: cytosine at position 131058184 of human chromosome 2; cytosine at position 36259383 of human chromosome 21; cytosine at position 180614858 of human chromosome 5; cytosine at position 18899483 of human chromosome 19; cytosine at position 190448126 of human chromosome 1; cytosine at position 2075777 of human chromosome 8; cytosine at position 218340518 of human chromosome 2; and cytosine at position 10415636 of human chromosome 6.

16. The in vitro method according to claim 1, which further comprising determining the methylation status of one or more CpG sites of the CAR T-cells obtained by transduction of the T cells in the isolated sample, the one or more CpG sites of CAR T cells selected from the group consisting of: cytosine at position 131058184 of human chromosome 2; cytosine at position 102242535 of human chromosome 10; cytosine at position 23015936 of human chromosome 20; cytosine at position 134571377 of human chromosome 10; cytosine at position 65294635 of human chromosome 8; cytosine at position 100879199 of human chromosome 15; cytosine at position 32353565 of human chromosome 11; cytosine at position 124132919 of human chromosome 9; cytosine at position 42299379 of human chromosome 19; cytosine at position 180614858 of human chromosome 5; cytosine at position 134457731 of human chromosome 10; cytosine at position 22634199 of human chromosome 10; cytosine at position 686450 of human chromosome 17; cytosine at position 90081872 of human chromosome 14; cytosine at position 127568850 of human chromosome 8; cytosine at position 94057587 of human chromosome 1; cytosine at position 134149184 of human chromosome 10, cytosine at position 17109239 of human chromosome 17; cytosine at position 36258423 of human chromosome 21; cytosine at position 132481826 of human chromosome 2, cytosine at position 104535854 of human chromosome 10; and cytosine at position 190448126 of human chromosome 1, all cytosine positions in human chromosomes according to the chromosome map and sequence entries of database UCSC Genome Browser on Human February 2009, GRCh37/hg19 assembly of the University of California Santa Cruz (UCSC).

17. The in vitro method according to claim 16, wherein the one or more CpG sites of CAR T cells selected from the group consisting of: cytosine at position 131058184 of human chromosome 2; cytosine at position 102242535 of human chromosome 10; cytosine at position 23015936 of human chromosome 20; cytosine at position 134571377 of human chromosome 10; and cytosine at position 65294635 of human chromosome 8.

18. The in vitro method according to claim 5, further comprising determining the methylation status of one or more CpG sites of the CAR T-cells obtained by transduction of the T cells in the isolated sample, the one or more CpG sites of CAR T cells selected from the group consisting of: cytosine al position 201123894 of human chromosome 1; cytosine at position 45505849 of human chromosome 3; cytosine at position 2075777 of human chromosome 8; cytosine at position 218340518 of human chromosome 2; cytosine at position 85637673 of human chromosome 2; cytosine at position 10415636 of human chromosome 6; cytosine at position 29990921 of human chromosome 14; cytosine at position 100879199 of human chromosome 15; cytosine at position 105648138 of human chromosome 10; cytosine at position 637813 of human chromosome 8; cytosine at position 110721138 of human chromosome 6; and cytosine at position 130516192 of human chromosome 12, all cytosine positions in human chromosomes according to the chromosome map and sequence entries of database UCSC Genome Browser on Human February 2009, GRCh37/hg19 assembly of the University of California Santa Cruz (UCSC).

19. (canceled)

20. A method of deciding and/or recommending whether to initiate an autologous CAR T-cell therapy for a subject suffering from B-cell malignancies, which method comprises carrying out the in vitro method as defined in claim 1; and wherein if the subject is determined to respond to CAR T-cell therapy, then the therapy is recommended.

21. The method according to claim 1, further comprising isolating an identified candidate CAR T-cell or a population thereof to obtain an isolated candidate CAR T-cell or population thereof and optionally expanding the isolated candidate CAR T-cell or population thereof to obtain an expanded candidate CAR T-cell or population thereof.

22-23. (canceled)
